(19) United States
(12) Patent Application Publication
(10) Pub. No.: US 2020/0386759 A1
(43) Pub. Date: Dec. 10, 2020

(54) ROBUST PANELS OF COLORECTAL CANCER BIOMARKERS

(71) Applicant: DISCERNDX, INC., Palo Alto, CA (US)

(72) Inventors: Bruce WILCOX, Palo Alto, CA (US); Lisa CRONER, Palo Alto, CA (US); Athit KAO, Palo Alto, CA (US); Jia YOU, Palo Alto, CA (US); Roslyn DILLON, Palo Alto, CA (US)

(21) Appl. No.: 16/769,544

(22) PCT Filed: Dec. 5, 2018
(86) PCT No.: PCT/US2018/064107

§ 371 Date: Jun. 3, 2020

Related U.S. Application Data
(60) Provisional application No. 62/594,941, filed on Dec. 5, 2017.

(51) Int. Cl. G01N 33/574; G16H 15/00; G16H 50/30

(52) U.S. Cl. CPC G01N 33/57419; G16H 15/00; G16H 50/30; C12Y 301/03048; G01N 2333/4745; G01N 2333/916; G01N 2333/4728

(57) ABSTRACT

Described herein are systems and methods for developing and utilizing assays for assessing health status such as colorectal cancer.

### CROSS-REFERENCE

This application claims the benefit of U.S. Prov. App. Ser. No. 62/594,941, filed Dec. 5, 2017, which is hereby explicitly incorporated herein by reference in its entirety.

### BACKGROUND

Over the past 20 years, mass spectrometry (MS) has emerged as a dynamic tool for proteomics-based biomarker discovery, providing more information than can be obtained from other high-throughput approaches. However, published biomarker candidates from MS studies often fail to translate to the clinic, when promising claims from original studies cannot be independently reproduced.

### SUMMARY

Provided herein are methods and systems that provide targeted proteomics workflows that effectively identify protein biomarkers associated with diseases such as, for example, colorectal cancer. The present disclosure recognizes that the failures of past mass spectrometry studies can be attributed to various shortcomings such as in study design, sample quality, assay robustness, assay reproducibility, and/or quality control. Accordingly, certain aspects of the methods and systems disclosed herein utilize quality and/or process control metrics and procedures to enhance predictive accuracy and consistency.

Provided herein are noninvasive methods of assessing a CRC status in an individual, for example using a blood sample of an individual. Some such methods comprise the steps of obtaining a circulating blood sample from the individual; obtaining a biomarker panel level for a biomarker panel comprising a list of proteins in the sample comprising A2GL, ALS, and PTPRJ, and also including individual age and gender as biomarkers to comprise panel information from said individual, and using said panel information to make a CRC health assessment. Some approaches comprise comparing said panel information from said individual to a reference panel information set corresponding to a known colorectal cancer status, such as at least one of no CRC, stage I CRC, Stage II CRC, stage III CRC, stage IV CRC, and more generally early CRC, advanced CRC; and categorizing said individual as having said colorectal cancer status if said individual's reference panel information does not differ significantly from said reference panel information set. Some approaches comprise using panel levels in an algorithm to obtain a panel score, and comparing the panel score to that of panel scores for at least one reference panel information set score corresponding to a known colorectal cancer status, such as at least one of no CRC, stage I CRC, Stage II CRC, stage III CRC, stage IV CRC, and more generally early CRC, advanced CRC; and categorizing said individual as having said colorectal cancer status if said individual's reference panel information does not differ significantly from said reference panel information set. Some approaches comprise using ratios of selected biomarkers relative to one another in an algorithm to obtain a panel score, and comparing the panel score to that of panel scores for at least one reference panel information set score corresponding to a known colorectal cancer status, such as at least one of no CRC, stage I CRC, Stage II CRC, stage III CRC, stage IV CRC, and more generally early CRC, advanced CRC; and categorizing said individual as having said colorectal cancer status if said individual's reference panel information does not differ significantly from said reference panel information set.

Some approaches comprise comparing said panel information from said individual to a reference panel information set corresponding to a known colorectal cancer status, such as at least one of no CRC, stage I CRC, Stage II CRC, stage III CRC, stage IV CRC, and more generally early CRC, advanced CRC; and categorizing said individual as having a CRC status different from said reference panel if said individual's reference panel information differs significantly from said reference panel information set. Some approaches comprise using panel levels in an algorithm to obtain a panel score, and comparing the panel score to that of panel scores for at least one reference panel information set score corresponding to a known colorectal cancer status, such as at least one of no CRC, stage I CRC, Stage II CRC, stage III CRC, stage IV CRC, and more generally early CRC, advanced CRC; and categorizing said individual as not having said colorectal cancer status if said individual's reference panel information differs significantly from said reference panel information set. Some approaches comprise using ratios of selected biomarkers relative to one another in an algorithm to obtain a panel score, and comparing the panel score to that of panel scores for at least one reference panel information set score corresponding to a known colorectal cancer status, such as at least one of no CRC, stage I CRC, Stage II CRC, stage III CRC, stage IV CRC, and more generally early CRC, advanced CRC; and categorizing said individual as not having said colorectal cancer status if said individual's reference panel information differs significantly from said reference panel information set.

Some CRC panels disclosed herein demonstrate a Validation Area Under curve (AUC), a parameter of panel test success, of at least 0.80, such as 0.81, 0.82, 0.83, 0.84, 0.85, 0.86, 0.87, 0.88, 0.89, 0.90, or greater than 0.90. In some cases, one observes a CRC AUC of 0.82 or about 0.82, and a Validation Sensitivity of 0.81 or about 0.81 and a validation specificity of 0.78 or about 0.78.

Also provided herein are noninvasive methods of assessing an advanced adenoma status in an individual, for example using a blood sample of an individual. Some such methods comprise the steps of obtaining a circulating blood sample from the individual; obtaining a biomarker panel level for a biomarker panel comprising a list of proteins in the sample comprising A2GL, ALS, and PTPRJ, and obtaining the age of the individual as biomarkers to comprise panel information from said individual, and using said panel information to make a CRC health assessment. Some approaches comprise comparing said panel information from said individual to a reference panel information set corresponding to a known AA status; and categorizing said individual as having said AA status if said individual's reference panel information does not differ significantly from said reference panel information set. Some approaches comprise using panel levels in an algorithm to obtain a panel score, and comparing the panel score to that of panel scores for at least one reference panel information set score corresponding to a known AA status; and categorizing said individual as having said AA status if said individual's reference panel information does not differ significantly from said reference panel information set. Some approaches comprise using ratios of selected biomarkers relative to one another in an algorithm to obtain a panel score, and comparing the panel score to that of panel scores for at least one reference panel information set score corresponding to a known AA status; and categorizing said individual as having said AA status if said individual's reference panel information does not differ significantly from said reference panel information set.

Some approaches comprise comparing said panel information from said individual to a reference panel information set corresponding to a known AA status; and categorizing said individual as having an AA status different from said reference panel if said individual's reference panel information differs significantly from said reference panel information set. Some approaches comprise using panel levels in an algorithm to obtain a panel score, and comparing the panel score to that of panel scores for at least one reference panel information set score corresponding to a known AA status; and categorizing said individual as not having said AA status if said individual's reference panel information differs significantly from said reference panel information set. Some approaches comprise using ratios of selected biomarkers relative to one another in an algorithm to obtain a panel score, and comparing the panel score to that of panel scores for at least one reference panel information set score corresponding to a known AA status; and categorizing said individual as not having said AA status if said individual's reference panel information differs significantly from said reference panel information set.

In light of the above and the disclosure herein, provided herein are methods, compositions, kits, computer readable media, and systems for the diagnosis and/or treatment of at least one of advanced colorectal adenoma and colorectal cancer. Through the methods and compositions provided herein, a sample is taken from an individual. In some cases the individual presents no symptoms of colorectal cancer, or advanced adenoma, or both colorectal cancer and adenoma. Some individuals are tested as part of routine health observation or monitoring. Alternately, some individuals are tested in relation to presenting at least one symptom of a colorectal health issue such as colorectal cancer, or advanced adenoma, or both colorectal cancer and adenoma. In some cases the individual is identified as being at risk of colorectal cancer, or advanced adenoma, or both colorectal cancer and adenoma. The sample is assayed to determine the accumulation levels of a panel of markers such as proteins, or proteins and age, or proteins and gender, or proteins and age and gender, for example a panel of markers comprising or consisting of the markers in panels disclosed herein. In many cases the panels comprise proteins that individually are known to play a role in indicating the presence of advanced colorectal adenoma or colorectal cancer, while in other cases the panels comprise a protein or proteins not know to correlate with advanced colorectal adenoma or colorectal cancer. However, in all cases the identification and accumulation of markers into a panel results in a level of specificity, sensitivity or specificity and sensitivity that substantially surpasses that of individual markers or smaller or less accurate sets of markers.

Additionally, methods, panels and other tests disclosed herein substantially surpass the sensitivity, specificity, or sensitivity and specificity of many commercially available tests, in particular many currently available blood-based tests. Methods, panels and other tests disclosed herein have the further benefit of being easily executed, such that an individual in need of gastrointestinal health evaluation test results is much more likely to have this test performed, rather than collecting a stool sample or having an invasive procedure such as a colonoscopy, for example. Panel accumulation levels are measured in a number of ways in various embodiments, for example through an antibody florescence binding assay or an ELISA assay, through mass spectroscopy analysis, through detection of florescence of an antibody set, or through alternate approaches to protein accumulation level quantification.

Panel accumulation levels are assessed through a number of approaches consistent with the disclosure herein. For example panel accumulation levels are compared to a positive control or negative control standard comprising at least one and up to 10, 100, or more than 100 standards of known colorectal health status, or to a model of advanced colorectal adenoma or colorectal cancer accumulation levels or of healthy accumulation levels, such that a prediction is made regarding an assayed individual's health status. Alternately or in combination, panel results are compared to a machine learning or other model trained on or built upon data obtained from known positive or known negative patient samples. In some cases, a panel assay result is accompanied by a recommendation regarding an intervention or an alternate verification of the panel assay results.

Accordingly, provided herein are biomarker panels and assays useful for the diagnosis and/or treatment of at least one of advanced colorectal adenoma and colorectal cancer.

Also provided herein are kits, comprising a computer readable medium described herein, and instructions for use of the computer readable medium.

A number of treatment regimens are contemplated herein and known to one of skill in the art, such as chemotherapy, administration of a biologic therapeutic agent, and surgical intervention such as low anterior resection or abdominoperineal resection, or ostomy.

Also provided herein are approaches for determining a panel of biomarkers suitable for assessing colorectal health status such as colorectal cancer, advanced colorectal adenoma, and/or stage of colorectal cancer.

Described herein is the development and experimental steps of a method for identifying biomarkers relevant to disease or health status. A number of approaches are consistent with the disclosure herein, such as large-scale dMRM-based workflow. A number of approaches include the use of at least one process control to evaluate aspects of the analytical instrumentation. In some cases, the method implements SST, using SIS peptide mixture and pooled plasma sample as reference material, or any combination thereof. In some cases, the approach instrumentation metrics that are evaluated include consistency of the response, carryover, retention time stability, signal-to-noise, or other suitable metrics. In certain instances, quality controls are used in the form of pooled plasma sample to monitor and if needed, correct the analytical variability during sample processing and analysis. Quality control metrics can be utilized to assess the sample and/or sample processing. The use of QC markers to provide information indicative of workflow or assay performance is consistent with the present disclosure and can include markers that undergo at least one of collection, storage, elution, processing, and analysis together with the sample.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows concurrent MRMs vs Retention Time.

FIG. 2 shows an example of CE optimization for a heavy transition.

FIG. 3 shows standard curves illustrating the range of transition assays observed.

FIG. 4 shows frequency histograms and summary statistics for metrics across 1357 transitions.

FIG. 5 shows standard deviations for flow-through peak AUCs for PQCs.

FIG. 6 shows RT shifts for all the 1552 heavy transitions for nine consecutive running days on one Agilent QQQ.

FIG. 7 shows PQC peak AUC CV pass rate over 176 QC heavy transitions across data collection dates.

FIG. 8 shows PQC peak AUC CV pass rate over 176 QC light transitions across data collection dates.

FIG. 9 shows a histogram of transition AUCs.

FIG. 10 shows algorithm selection replaced after manual review.

FIG. 11 shows a peptide that was detected in depleted flow-through collection by LC-MS/MS.

FIG. 12 shows standard deviations for flow-through peak AUCs for PQCs indicating consistent immuno-depletion over time.

FIG. 13 shows molecular features and miscleavage rates across sample plates.

FIG. 14 shows 5-point curve data for heavy peak AUCs of 176 pre-selected QC transitions.

FIG. 15 shows a diagram of various steps that can be utilized to generate reliable targeted mass spectrometry results.

FIG. 16 shows characteristics and performances of three validated CRC vs non-CRC classifiers.

FIG. 17 characteristics and validation outcomes of the 58 simple grid builds. The columns “dx,” “build group,” and “build” apply to the full grid of classifiers examined in each build, and were used to arrange the table. The remaining columns give characteristics of the best classifier found in each grid. “Pre-noc median merged test auc” is the pre-NoC CRC vs NCNF discovery set AUC. “# transitions meeting all quality metrics” is the number of transitions that had complete measures, had good quality peaks, and were judged as quantitative assays. Blue and orange highlights indicate classifiers for which NoC analyses were performed, with orange rows indicating those for which validation was also attempted. In the “note” column, “age” indicates that the classifier AUC was statistically indistinguishable from the univariate age AUC in the validation set.

FIG. 18 shows the validation set ROC for model 28. Red 1801, orange 1802, and green 1803 dots are sens/spec 0.80/0.80, 0.80/0.75, and observed, respectively.

FIG. 19 shows the validation set ROC for model 40. Red 1901, orange 1902, and green 1903 dots are sens/spec 0.80/0.80, 0.80/0.75, and observed, respectively.

FIG. 20 shows the validation set ROC for model 52. Red 2001, orange 2002, and green 2003 dots are sens/spec 0.80/0.80, 0.80/0.75, and observed, respectively.

### DETAILED DESCRIPTION

Provided herein are noninvasive methods of assessing a health status in an individual, for example colorectal cancer status using a biological sample of the individual. Some such methods comprise the steps of obtaining a circulating blood sample from the individual; obtaining a biomarker panel level for a biomarker panel comprising a list of proteins in the sample selected from Table 1, and using said panel information to make a CRC health assessment. In some cases, individual age and/or gender are also selected as biomarkers to comprise panel information from said individual. Some approaches comprise comparing said panel information from said individual to a reference panel information set corresponding to a known colorectal cancer status, such as at least one of no CRC, stage I CRC, Stage II CRC, stage III CRC, stage IV CRC, and more generally early CRC, advanced CRC; and categorizing said individual as having said colorectal cancer status if said individual's reference panel information does not differ significantly from said reference panel information set. Some approaches comprise using panel levels in an algorithm to obtain a panel score, and comparing the panel score to that of panel scores for at least one reference panel information set score corresponding to a known colorectal cancer status, such as at least one of no CRC, stage I CRC, Stage II CRC, stage III CRC, stage IV CRC, and more generally early CRC, advanced CRC; and categorizing said individual as having said colorectal cancer status if said individual's reference panel information does not differ significantly from said reference panel information set. Some approaches comprise using ratios of selected biomarkers relative to one another in an algorithm to obtain a panel score, and comparing the panel score to that of panel scores for at least one reference panel information set score corresponding to a known colorectal cancer status, such as at least one of no CRC, stage I CRC, Stage II CRC, stage III CRC, stage IV CRC, and more generally early CRC, advanced CRC; and categorizing said individual as having said colorectal cancer status if said individual's reference panel information does not differ significantly from said reference panel information set.

Biomarker panels as disclosed herein share a property that sensitive, specific conclusions regarding an individual's colorectal health are made using protein level information derived from circulating blood, alone or in combination with other information such as an individual's age, gender, health history or other characteristics. A benefit of the present biomarker panels is that they provide a sensitive, specific colorectal health assessment using conveniently, noninvasively obtained samples. There is no need to rely upon data obtained from an intrusive abdominal assay such as a colonoscopy or a sigmoidoscopy, or from stool sample material. As a result compliance rates are substantially higher, and colorectal health issues are more easily recognized early in their progression, so that they may be more efficiently treated. Ultimately, the effect of this benefit is measured in lives saved, and is substantial.

Biomarker panels as disclosed herein are selected such that their predictive value as panels is substantially greater than the predictive value of their individual members. Panel members generally do not co-vary with one another, such that panel members provide independent contributions to the panel's overall health signal. Accordingly, a panel is able to substantially outperform the performance of any individual constituent indicative of an individual's colorectal health status, such that a commercially and medicinally relevant degree of confidence (such as sensitivity, specificity or sensitivity and specificity) is obtained. Thus, in the panels as disclosed herein, multiple panel members indicative of a health issue provide a much stronger signal than is found, for example in a panel wherein two or more members rise or fall in strict concert such that the signal derived therefrom is effectively a single signal, repeated twice. Accordingly, panels as disclosed herein are robust to variation in single constituent measurements. For example because panel members vary independently of one another, panels herein often indicate a health risk despite the fact that one or more than one individual members of the panel would not indicate that the health risk is present if measured alone. In some cases, panels herein indicate a health risk at a significant level of confidence despite the fact that no individual panel member indicates the health risk at a significant level of confidence on its own. In some cases, panels herein indicate a health risk at a significant level of confidence despite the fact that at least one individual member indicates at a significant level of confidence that the health risk is not present.

Biomarkers consistent with the panels herein comprise biological molecules that circulate in the bloodstream of an individual, such as proteins. Readily available information including demographic information such as individual's age or gender is also included in some cases. Physiological information including weight, height, body mass index, as well as other easily measured or obtained information is also eligible as a marker. In particular, some panels herein rely upon age, gender, or age and gender as biomarkers.

Common to many biomarkers herein is the ease with which they are assayed in an individual. Biomarkers herein are readily obtained by a blood draw from an artery or vein of an individual, or are obtained via interview or by simple biometric analysis. A benefit of the ease with which biomarkers herein are obtained is that invasive assays such as colonoscopy or sigmoidoscopy are not required for biomarker measurement. Similarly, stool samples are not required for biomarker determination. As a result, panel information as disclosed herein is often readily obtained through a blood draw in combination with a visit to a doctor's office. Compliance rates are accordingly substantially higher than are compliance rates for colorectal health assays involving stool samples or invasive procedures.

Exemplary panels disclosed herein comprise circulating proteins or fragments thereof that are recognizably or uniquely mapped to their parent protein, and in some cases comprise a readily obtained biomarker such as an individual's age.

### Panel Constituents

Some biomarker panels comprise some or all of the protein markers recited herein, subsets thereof or listed markers in combination with additional markers or biological parameters. A lead biomarker panel relevant to colorectal cancer and/or advanced adenoma assessment comprises at least 1, 2, 3, or 4 markers, up to the full list, alone or in combination with additional markers, said list selected from the following: A2GL, ACTBM, ALS, APOC4, APOE, APOL1, CHLE, GELS, I10R1, ITIH2, KAIN, PON1, PTPRJ, SPP24, TFR1, TNF15, IBP3, THRB, GUC2A, LYNX1, PREX2, RET4, and also including age and optionally gender as biomarkers. In some cases, the ratio between a protein marker and age is utilized as a feature in the panel for making a CRC assessment, for example, PTPRJ/age and/or ALS/age ratios. As used herein, a ratio can include a ratio between a peptide fragment of a protein marker and a demographic such as age. A peptide/marker ratio can include a ratio between at least one peptide derived from any of A2GL, ACTBM, ALS, APOC4, APOE, APOL1, CHLE, GELS, I10R1, ITIH2, KAIN, PON1, PTPRJ, SPP24, TFR1, TNF15, IBP3, THRB, GUC2A, LYNX1, PREX2, and RET4 and a demographic such as age. Examples of peptide/age ratios can be found in the working examples described herein. Non-limiting examples of Another lead biomarker panel relevant to colorectal cancer and/or advanced adenoma assessment comprises markers selected from the following: A2GL, ACTBM, ALS, APOC4, APOE, APOL1, CHLE, I10R1, ITIH2, KAIN, PON1, PTPRJ, SPP24, TFR1, TNF15, and also including age of the individual as a biomarker. Another lead biomarker panel, or a combination of biomarker panels having colorectal cancer and advanced adenoma assessment capabilities comprises markers selected from the following: A2GL, ALS, PTPRJ, and age, or a subset thereof optionally having at least one individual marker excluded or replaced with one or more markers. Another lead biomarker panel, or a combination of biomarker panels having colorectal cancer and advanced adenoma assessment capabilities comprises markers selected from the following: A2GL, ALS, GELS, PTPRJ, and age, or a subset thereof optionally having at least one individual marker excluded or replaced with one or more markers. In some cases, a CRC biomarker panel comprises one or more ratios of a protein marker relative to age.

Often, it is convenient or efficient to combine a CRC biomarker panel and an advanced adenoma panel into a single kit or a single biomarker panel. In these cases, one sees a kit comprising three biomarkers, or a subset or larger set thereof, including A2GL, ALS, and PTPRJ, if included, is informative as to both colorectal cancer status and advanced adenoma status, particularly in combination with information regarding patient age. Alternate and variant colorectal cancer biomarker panels are listed below.

Much like the panel discussed above, these panels, or subsets or additions, are used alone or in combination with the above-mentioned advanced adenoma panel, optionally using markers such as A2GL, ACTBM, ALS, APOC4, APOE, APOL1, CHLE, GELS, I10R1, ITIH2, KAIN, PON1, PTPRJ, SPP24, TFR1, TNF15, IBP3, THRB, GUC2A, LYNX1, PREX2, RET4, and also in combination with age, to be indicative of colorectal cancer status and/or advanced adenoma.

Accordingly, disclosed herein are colorectal health assessment panels comprising the biomarkers mentioned above. Panels comprise at least 1, 2, 3, 4, 5, 6, 7, 8, 9, 10, 11, 12, 13, 14, 15, 16, 17, 18, 19, 20, 21, or 22, or more than 22 of the biomarkers mentioned herein such as, for example, those listed in Table 1.

### Biomarkers

In some cases, biomarker panels described herein comprise at least three biomarkers. The biomarkers can be selected from the group of identifiable polypeptides or fragments of the 22 protein biomarkers listed in Table 1, optionally used in combination with age and/or gender. Any of the biomarkers described herein can be protein biomarkers. Furthermore, the group of biomarkers in this example can in some cases additionally comprise polypeptides with the characteristics found in Table 1. In some cases, the ratio of one or more protein biomarkers described herein (e.g., one or more proteotypic peptides evaluated by mass spectrometry) to another biomarker such as age is utilized in making the assessment of health status.

Exemplary protein biomarkers and, when available, their human amino acid sequences, are listed in Table 1, below. Protein biomarkers comprise full length molecules of the polypeptide sequences of Table 1, as well as uniquely identifiable fragments of the polypeptide sequences of Table 1. Markers can be but do not need to be full length to be informative. In many cases, so long as a fragment is uniquely identifiable as being derived from or representing a polypeptide of Table 1, it is informative for purposes herein.

TABLE 1
Biomarkers and corresponding Descriptors

| No./ | | |
|---|---|---|
| Protein Name/ | | |
| Protein | | |
| Symbol and | Protein Sequence | |
| Synonyms/ | (N- to C-terminal single-letter amino acid | SEQ |
| Uniprot ID | sequence) or other Descriptor of Biomarker | ID NO. |
| No. 1/ | MSSWSRQRPKSPGGIQPHVSRTLFLLLLLAASAWGVTLSPKD | 1 |
| Leucine Rich | CQVFRSDHGSSISCQPPAEIPGYLPADTVHLAVEFFNLTHLP | |
| Alpha-2- | ANLLQGASKLQELHLSSNGLESLSPEFLRPVPQLRVLDLTRN | |
| Glycoprotein | ALTGLPPGLFQASATLDTLVLKENQLEVLEVSWLHGLKALGH | |
| 1/A2GL, | LDLSGNRLRKLPPGLLANFTLLRTLDLGENQLETLPPDLLRG | |
| LRG1/ | PLQLERLHLEGNKLQVLGKDLLLPQPDLRYLFLNGNKLARVA | |
| P02750 | AGAFQGLRQLDMLDLSNNSLASVPEGLWASLGQPNWDMRDGF | |
| | DISGNPWICDQNLSDLYRWLQAQKDKMFSQNDTRCAGPEAVK | |
| | GQTLLAVAKSQ | |
| No. 2/ | MDDDTAVLVIDNGSGMCKAGFAGDDAPQAVFPSIVGRPRHQG | 2 |
| POTE Ankyrin | MMEGMHQKESYVGKEAQSKRGMLTLKYPMEHGIITNWDDMEK | |
| Domain | IWHHTFYNELRVAPEEHPILLTEAPLNPKANREKMTQIMFET | |
| Family, | FNTPAMYVAIQAVLSLYTSGRTTGIVMDSGDGFTHTVPIYEG | |
| Member K, | NALPHATLRLDLAGRELTDYLMKILTERGYRFTTTAEQEIVR | |
| Pseudogene12/ | DIKEKLCYVALDSEQEMAMAASSSSVEKSYELPDGQVITIGN | |
| ACTBM, | ERFRCPEALFQPCFLGMESCGIHKTTFNSIVKSDVDIRKDLY | |
| ACTBL3, | TNTVLSGGTTMYPGIAHRMQKEITALAPSIMKIKIIAPPKRK | |
| POTEKP/Q9BYX7 | YSVWVGGSILASLSTFQQMWISKQEYDESGPSIVHRKCF | |
| No. 3/Insulin | MALRKGGLALALLLLSWVALGPRSLEGADPGTPGEAEGPACP | 3 |
| Like Growth | AACVCSYDDDADELSVFCSSRNLTRLPDGVPGGTQALWLDGN | |
| Factor | NLSSVPPAAFQNLSSLGFLNLQGGQLGSLEPQALLGLENLCH | |
| Binding | LHLERNQLRSLALGTFAHTPALASLGLSNNRLSRLEDGLFEG | |
| Protein Acid | LGSLWDLNLGWNSLAVLPDAAFRGLGSLRELVLAGNRLAYLQ | |
| Labile Subunit/ | PALFSGLAELRELDLSRNALRAIKANVFVQLPRLQKLYLDRN | |
| ALS, | LIAAVAPGAFLGLKALRWLDLSHNRVAGLLEDTFPGLLGLRV | |
| IGFALS, | LRLSHNAIASLRPRTFKDLHFLEELQLGHNRIRQLAERSFEG | |
| ACLSD/ | LGQLEVLTLDHNQLQEVKAGAFLGLTNVAVMNLSGNCLRNLP | |
| P35858 | EQVFRGLGKLHSLHLEGSCLGRIRPHTFTGLSGLRRLFLKDN | |
| | GLVGIEEQSLWGLAELLELDLTSNQLTHLPHRLFQGLGKLEY | |
| | LLLSRNRLAELPADALGPLQRAFWLDVSHNRLEALPNSLLAP | |
| | LGRLRYLSLRNNSLRTFTPQPPGLERLWLEGNPWDCGCPLKA | |
| | LRDFALQNPSAVPRFVQAICEGDDCQPPAYTYNNITCASPPE | |
| | VVGLDLRDLSEAHFAPC | |
| No. 4/ | MSLLRNRLQALPALCLCVLVLACIGACQPEAQEGTLSPPPKL | 4 |
| Apolipoprotein | KMSRWSLVRGRMKELLETVVNRTRDGWQWFWSPSTFRGFMQT | |
| C4/ | YYDDHLRDLGPLTKAWFLESKDSLLKKTHSLCPRLVCGDKDQ | |
| APOC4, | G | |
| APOC-IV/ | | |
| P55056 | | |
| No. 5/ | MKVLWAALLVTFLAGCQAKVEQAVETEPEPELRQQTEWQSGQ | 5 |
| Apolipoprotein | RWELALGRFWDYLRWVQTLSEQVQEELLSSQVTQELRALMDE | |
| E/APOE, | TMKELKAYKSELEEQLTPVAEETRARLSKELQAAQARLGADM | |
| LPG, AD2/ | EDVCGRLVQYRGEVQAMLGQSTEELRVRLASHLRKLRKRLLR | |
| P02649 | DADDLQKRLAVYQAGAREGAERGLSAIRERLGPLVEQGRVRA | |
| | ATVGSLAGQPLQERAQAWGERLRARMEEMGSRTRDRLDEVKE | |
| | QVAEVRAKLEEQAQQIRLQAEAFQARLKSWFEPLVEDMQRQW | |
| | AGLVEKVQAAVGTSAAPVPSDNH | |
| No. 6/ | MEGAALLRVSVLCIWMSALFLGVGVRAEEAGARVQQNVPSGT | 6 |
| Apolipoprotein | DTGDPQSKPLGDWAAGTMDPESSIFIEDAIKYFKEKVSTQNL | |
| L1/APOL, | LLLLTDNEAWNGFVAAAELPRNEADELRKALDNLARQMIMKD | |
| APOL1/ | KNWHDKGQQYRNWFLKEFPRLKSELEDNIRRLRALADGVQKV | |
| O14791 | HKGTTIANVVSGSLSISSGILTLVGMGLAPFTEGGSLVLLEP | |
| | GMELGITAALTGITSSTMDYGKKWWTQAQAHDLVIKSLDKLK | |
| | EVREFLGENISNFLSLAGNTYQLTRGIGKDIRALRRARANLQ | |
| | SVPHASASRPRVTEPISAESGEQVERVNEPSILEMSRGVKLT | |
| | DVAPVSFFLVLDVVYLVYESKHLHEGAKSETAEELKKVAQEL | |
| | EEKLNILNNNYKILQADQEL | |
| No. 7/ | MHSKVTIICIRFLFWFLLLCMLIGKSHTEDDIIIATKNGKVR | 7 |
| cholinesterase/ | GMNLTVFGGTVTAFLGIPYAQPPLGRLRFKKPQSLTKWSDIW | |
| CHLE, | NATKYANSCCQNIDQSFPGFHGSEMWNPNTDLSEDCLYLNVW | |
| BCHE, CHE1/ | IPAPKPKNATVLIWIYGGGFQTGTSSLHVYDGKFLARVERVI | |
| P06276 | VVSMNYRVGALGFLALPGNPEAPGNMGLFDQQLALQWVQKNI | |
| | AAFGGNPKSVTLFGESAGAASVSLHLLSPGSHSLFTRAILQS | |
| | GSFNAPWAVTSLYEARNRTLNLAKLTGCSRENETEIIKCLRN | |
| | KDPQEILLNEAFVVPYGTPLSVNFGPTVDGDFLTDMPDILLE | |
| | LGQFKKTQILVGVNKDEGTAFLVYGAPGFSKDNNSIITRKEF | |
| | QEGLKIFFPGVSEFGKESILFHYTDWVDDQRPENYREALGDV | |
| | VGDYNFICPALEFTKKFSEWGNNAFFYYFEHRSSKLPWPEWM | |
| | GVMHGYEIEFVFGLPLERRDNYTKAEEILSRSIVKRWANFAK | |
| | YGNPNETQNNSTSWPVFKSTEQKYLTLNTESTRIMTKLRAQQ | |
| | CRFWTSFFPKVLEMTGNIDEAEWEWKAGFHRWNNYMMDWKNQ | |
| | FNDYTSKKESCVGL | |
| No. 8/ | MAPHRPAPALLCALSLALCALSLPVRAATASRGASQAGAPQG | 8 |
| gelsolin/ | RVPEARPNSMVVEHPEFLKAGKEPGLQIWRVEKFDLVPVPTN | |
| GSN, GELS, | LYGDFFTGDAYVILKTVQLRNGNLQYDLHYWLGNECSQDESG | |
| ADF/P06396 | AAAIFTVQLDDYLNGRAVQHREVQGFESATFLGYFKSGLKYK | |
| | KGGVASGFKHVVPNEVVVQRLFQVKGRRVVRATEVPVSWESF | |
| | NNGDCFILDLGNNIHQWCGSNSNRYERLKATQVSKGIRDNER | |
| | SGRARVHVSEEGTEPEAMLQVLGPKPALPAGTEDTAKEDAAN | |
| | RKLAKLYKVSNGAGTMSVSLVADENPFAQGALKSEDCFILDH | |
| | GKDGKIFVWKGKQANTEERKAALKTASDFITKMDYPKQTQVS | |
| | VLPEGGETPLFKQFFKNWRDPDQTDGLGLSYLSSHIANVERV | |
| | PFDAATLHTSTAMAAQHGMDDDGTGQKQIWRIEGSNKVPVDP | |
| | ATYGQFYGGDSYIILYNYRHGGRQGQIIYNWQGAQSTQDEVA | |
| | ASAILTAQLDEELGGTPVQSRVVQGKEPAHLMSLFGGKPMII | |
| | YKGGTSREGGQTAPASTRLFQVRANSAGATRAVEVLPKAGAL | |
| | NSNDAFVLKTPSAAYLWVGTGASEAEKTGAQELLRVLRAQPV | |
| | QVAEGSEPDGFWEALGGKAAYRTSPRLKDKKMDAHPPRLFAC | |
| | SNKIGRFVIEEVPGELMQEDLATDDVMLLDTWDQVFVWVGKD | |
| | SQEEEKTEALTSAKRYIETDPANRDRRTPITVVKQGFEPPSF | |
| | VGWFLGWDDDYWSVDPLDRAMAELAA | |
| No. 9/ | MLPCLVVLLAALLSLRLGSDAHGTELPSPPSVWFEAEFFHHI | 9 |
| Interleukin 10 | LHWTPIPNQSESTCYEVALLRYGIESWNSISNCSQTLSYDLT | |
| Receptor | AVTLDLYHSNGYRARVRAVDGSRHSNWTVTNTRFSVDEVTLT | |
| Subunit Alpha/ | VGSVNLEIHNGFILGKIQLPRPKMAPANDTYESIFSHFREYE | |
| IL10R, | IAIRKVPGNFTFTHKKVKHENFSLLTSGEVGEFCVQVKPSVA | |
| IL10RA, | SRSNKGMWSKEECISLTRQYFTVTNVIIFFAFVLLLSGALAY | |
| I10R1/ | CLALQLYVRRRKKLPSVLLFKKPSPFIFISQRPSPETQDTIH | |
| Q13651 | PLDEEAFLKVSPELKNLDLHGSTDSGFGSTKPSLQTEEPQFL | |
| | LPDPHPQADRTLGNREPPVLGDSCSSGSSNSTDSGICLQEPS | |
| | LSPSTGPTWEQQVGSNSRGQDDSGIDLVQNSEGRAGDTQGGS | |
| | ALGHHSPPEPEVPGEEDPAAVAFQGYLRQTRCAEEKATKTGC | |
| | LEEESPLTDGLGPKFGRCLVDEAGLHPPALAKGYLKQDPLEM | |
| | TLASSGAPTGQWNQPTEEWSLLALSSCSDLGISDWSFAHDLA | |
| | PLGCVAAPGGLLGSFNSDLVTLPLISSLQSSE | |
| No. 10/Inter- | MKRLTCFFICFFLSEVSGFEIPINGLSEFVDYEDLVELAPGK | 10 |
| Alpha-Trypsin | FQLVAENRRYQRSLPGESEEMMEEVDQVTLYSYKVQSTITSR | |
| Inhibitor | MATTMIQSKVVNNSPQPQNVVFDVQIPKGAFISNFSMTVDGK | |
| Heavy Chain | TFRSSIKEKTVGRALYAQARAKGKTAGLVRSSALDMENFRTE | |
| 2/ITIH2/ | VNVLPGAKVQFELHYQEVKWRKLGSYEHRIYLQPGRLAKHLE | |
| P19823 | VDVWVIEPQGLRFLHVPDTFEGHFDGVPVISKGQQKAHVSFK | |
| | PTVAQQRICPNCRETAVDGELVVLYDVKREEKAGELEVFNGY | |
| | FVHFFAPDNLDPIPKNILFVIDVSGSMWGVKMKQTVEAMKTI | |
| | LDDLRAEDHFSVIDFNQNIRTWRNDLISATKTQVADAKRYIE | |
| | KIQPSGGTNINEALLRAIFILNEANNLGLLDPNSVSLIILVS | |
| | DGDPTVGELKLSKIQKNVKENIQDNISLFSLGMGFDVDYDFL | |
| | KRLSNENHGIAQRIYGNQDTSSQLKKFYNQVSTPLLRNVQFN | |
| | YPHTSVTDVTQNNFHNYFGGSEIVVAGKFDPAKLDQIESVIT | |
| | ATSANTQLVLETLAQMDDLQDFLSKDKHADPDFTRKLWAYLT | |
| | INQLLAERSLAPTAAAKRRITRSILQMSLDHHIVTPLTSLVI | |
| | ENEAGDERMLADAPPQDPSCCSGALYYGSKVVPDSTPSWANP | |
| | SPTPVISMLAQGSQVLESTPPPHVMRVENDPHFIIYLPKSQK | |
| | NICFNIDSEPGKILNLVSDPESGIVVNGQLVGAKKPNNGKLS | |
| | TYFGKLGFYFQSEDIKIEISTETITLSHGSSTFSLSWSDTAQ | |
| | VTNQRVQISVKKEKVVTITLDKEMSFSVLLHRVWKKHPVNVD | |
| | FLGIYIPPTNKFSPKAHGLIGQFMQEPKIHIFNERPGKDPEK | |
| | PEASMEVKGQKLIITRGLQKDYRTDLVFGTDVTCWFVHNSGK | |
| | GFIDGHYKDYFVPQLYSFLKRP | |
| No. 11/ | MHLIDYLLLLLVGLLALSHGQLHVEHDGESCSNSSHQQILET | 11 |
| Serpin Family | GEGSPSLKIAPANADFAFRFYYLIASETPGKNIFFSPLSISA | |
| A Member 4/ | AYAMLSLGACSHSRSQILEGLGFNLTELSESDVHRGFQHLLH | |
| KAIN, | TLNLPGHGLETRVGSALFLSHNLKFLAKFLNDTMAVYEAKLF | |
| SERPINA4, | HTNFYDTVGTIQLINDHVKKETRGKIVDLVSELKKDVLMVLV | |
| KST, PI4/ | NYIYFKALWEKPFISSRTTPKDFYVDENTTVRVPMMLQDQEH | |
| P29622 | HWYLHDRYLPCSVLRMDYKGDATVFFILPNQGKMREIEEVLT | |
| | PEMLMRWNNLLRKRNFYKKLELHLPKFSISGSYVLDQILPRL | |
| | GFTDLFSKWADLSGITKQQKLEASKSFHKATLDVDEAGTEAA | |
| | AATSFAIKFFSAQTNRHILRFNRPFLVVIFSTSTQSVLFLGK | |
| | VVDPTKP | |
| No. 12/ | MAKLIALTLLGMGLALFRNHQSSYQTRLNALREVQPVELPNC | 12 |
| Paraoxonase 1/ | NLVKGIETGSEDLEILPNGLAFISSGLKYPGIKSFNPNSPGK | |
| PON1, ESA, | ILLMDLNEEDPTVLELGITGSKFDVSSFNPHGISTFTDEDNA | |
| MVCD5/ | MYLLVVNHPDAKSTVELFKFQEEEKSLLHLKTIRHKLLPNLN | |
| P27169 | DIVAVGPEHFYGTNDHYFLDPYLQSWEMYLGLAWSYVVYYSP | |
| | SEVRVVAEGFDFANGINISPDGKYVYIAELLAHKIHVYEKHA | |
| | NWTLTPLKSLDFNTLVDNISVDPETGDLWVGCHPNGMKIFFY | |
| | DSENPPASEVLRIQNILTEEPKVTQVYAENGTVLQGSTVASV | |
| | YKGKLLIGTVFHKALYCEL | |
| No. 13/ | MKPAAREARLPPRSPGLRWALPLLLLLLRLGQILCAGGTPSP | 13 |
| Protein | IPDPSVATVATGENGITQISSTAESFHKQNGTGTPQVETNTS | |
| Tyrosine | EDGESSGANDSLRTPEQGSNGTDGASQKTPSSTGPSPVFDIK | |
| Phosphatase, | AVSISPTNVILTWKSNDTAASEYKYVVKHKMENEKTITVVHQ | |
| Receptor Type | PWCNITGLRPATSYVFSITPGIGNETWGDPRVIKVITEPIPV | |
| J/PTPRJ, | SDLRVALTGVRKAALSWSNGNGTASCRVLLESIGSHEELTQD | |
| DEP1, | SRLQVNISGLKPGVQYNINPYLLQSNKTKGDPLGTEGGLDAS | |
| CD148, SCC1/ | NTERSRAGSPTAPVHDESLVGPVDPSSGQQSRDTEVLLVGLE | |
| Q12913 | PGTRYNATVYSQAANGTEGQPQAIEFRTNAIQVFDVTAVNIS | |
| | ATSLTLIWKVSDNESSSNYTYKIHVAGETDSSNLNVSEPRAV | |
| | IPGLRSSTFYNITVCPVLGDIEGTPGFLQVHTPPVPVSDFRV | |
| | TVVSTTEIGLAWSSHDAESFQMHITQEGAGNSRVEITTNQSI | |
| | IIGGLFPGTKYCFEIVPKGPNGTEGASRTVCNRTVPSAVFDI | |
| | HVVYVTTTEMWLDWKSPDGASEYVYHLVIESKHGSNHTSTYD | |
| | KAITLQGLIPGTLYNITISPEVDHVWGDPNSTAQYTRPSNVS | |
| | NIDVSTNTTAATLSWQNFDDASPTYSYCLLIEKAGNSSNATQ | |
| | VVTDIGITDATVTELIPGSSYTVEIFAQVGDGIKSLEPGRKS | |
| | FCTDPASMASFDCEVVPKEPALVLKWTCPPGANAGFELEVSS | |
| | GAWNNATHLESCSSENGTEYRTEVTYLNFSTSYNISITTVSC | |
| | GKMAAPTRNTCTTGITDPPPPDGSPNITSVSHNSVKVKFSGF | |
| | EASHGPIKAYAVILTTGEAGHPSADVLKYTYEDFKKGASDTY | |
| | VTYLIRTEEKGRSQSLSEVLKYEIDVGNESTTLGYYNGKLEP | |
| | LGSYRACVAGFTNITFHPQNKGLIDGAESYVSFSRYSDAVSL | |
| | PQDPGVICGAVFGCIFGALVIVTVGGFIFWRKKRKDAKNNEV | |
| | SFSQIKPKKSKLIRVENFEAYFKKQQADSNCGFAEEYEDLKL | |
| | VGISQPKYAAELAENRGKNRYNNVLPYDISRVKLSVQTHSTD | |
| | DYINANYMPGYHSKKDFIATQGPLPNTLKDFWRMVWEKNVYA | |
| | IIMLTKCVEQGRTKCEEYWPSKQAQDYGDITVAMTSEIVLPE | |
| | WTIRDFTVKNIQTSESHPLRQFHFTSWPDHGVPDTTDLLINF | |
| | RYLVRDYMKQSPPESPILVHCSAGVGRTGTFIAIDRLIYQIE | |
| | NENTVDVYGIVYDLRMEIRPLMVQTEDQYVFLNQCVLDIVRS | |
| | QKDSKVDLIYQNTTAMTIYENLAPVTTFGKTNGYIA | |
| No. 14/ | MISRMEKMTMMMKILIMFALGMNYWSCSGFPVYDYDPSSLRD | 14 |
| Secreted | ALSASVVKVNSQSLSPYLFRAFRSSLKRVEVLDENNLVMNLE | |
| Phospho- | FSIRETTCRKDSGEDPATCAFQRDYYVSTAVCRSTVKVSAQQ | |
| protein 24/ | VQGVHARCSWSSSTSESYSSEEMIFGDMLGSHKWRNNYLFGL | |
| SPP24, SPP2/ | ISDESISEQFYDRSLGIMRRVLPPGNRRYPNHRHRARINTDF | |
| Q13103 | E | |
| No. 15/ | MMDQARSAFSNLFGGEPLSYTRFSLARQVDGDNSHVEMKLAV | 15 |
| Transferrin | DEEENADNNTKANVTKPKRCSGSICYGTIAVIVFFLIGFMIG | |
| Receptor | YLGYCKGVEPKTECERLAGTESPVREEPGEDFPAARRLYWDD | |
| Protein 1/ | LKRKLSEKLDSTDFTGTIKLLNENSYVPREAGSQKDENLALY | |
| TFR1, TFR, | VENQFREFKLSKVWRDQHFVKIQVKDSAQNSVIIVDKNGRLV | |
| TFRC/ | YLVENPGGYVAYSKAATVTGKLVHANFGTKKDFEDLYTPVNG | |
| P02786 | SIVIVRAGKITFAEKVANAESLNAIGVLIYMDQTKFPIVNAE | |
| | LSFFGHAHLGTGDPYTPGFPSFNHTQFPPSRSSGLPNIPVQT | |
| | ISRAAAEKLFGNMEGDCPSDWKTDSTCRMVTSESKNVKLTVS | |
| | NVLKEIKILNIFGVIKGFVEPDHYVVVGAQRDAWGPGAAKSG | |
| | VGTALLLKLAQMFSDMVLKDGFQPSRSIIFASWSAGDFGSVG | |
| | ATEWLEGYLSSLHLKAFTYINLDKAVLGTSNFKVSASPLLYT | |
| | LIEKTMQNVKHPVTGQFLYQDSNWASKVEKLTLDNAAFPFLA | |
| | YSGIPAVSFCFCEDTDYPYLGTTMDTYKELIERIPELNKVAR | |
| | AAAEVAGQFVIKLTHDVELNLDYERYNSQLLSFVRDLNQYRA | |
| | DIKEMGLSLQWLYSARGDFFRATSRLTTDFGNAEKTDRFVMK | |
| | KLNDRVMRVEYHFLSPYVSPKESPFRHVFWGSGSHTLPALLE | |
| | NLKLRKQNNGAFNETLFRNQLALATWTIQGAANALSGDVWDI | |
| | DNEF | |
| No. 16/TNF | MAEDLGLSFGETASVEMLPEHGSCRPKARSSSARWALTCCLV | 16 |
| Superfamily | LLPFLAGLTTYLLVSQLRAQGEACVQFQALKGQEFAPSHQQV | |
| Member 15/ | YAPLRADGDKPRAHLTVVRQTPTQHFKNQFPALHWEHELGLA | |
| TNF15, | FTKNRMNYTNKFLLIPESGDYFIYSQVTFRGMTSECSEIRQA | |
| TNFSF15, | GRPNKPDSITVVITKVTDSYPEPTQLLMGTKSVCEVGSNWFQ | |
| TL1A, | PIYLGAMFSLQEGDKLMVNVSDISLVDYTKEDKTFFGAFLL | |
| TNLG1B, | | |
| VEGI/ | | |
| O95150 | | |
| No. 17/ | MQRARPTLWAAALTLLVLLRGPPVARAGASSAGLGPVVRCEP | 17 |
| Insulin Like | CDARALAQCAPPPAVCAELVREPGCGCCLTCALSEGQPCGIY | |
| Growth Factor | TERCGSGLRCQPSPDEARPLQALLDGRGLCVNASAVSRLRAY | |
| Binding | LLPAPPAPGNASESEEDRSAGSVESPSVSSTHRVSDPKFHPL | |
| Protein 3/ | HSKIIIIKKGHAKDSQRYKVDYESQSTDTQNFSSESKRETEY | |
| IBP3, | GPCRREMEDTLNHLKFLNVLSPRGVHIPNCDKKGFYKKKQCR | |
| IGFBP3/ | PSKGRKRGFCWCVDKYGQPLPGYTTKGKEDVHCYSMQSK | |
| P17936 | | |
| No. 18/ | MTPNSMTENGLTAWDKPKHCPDREHDWKLVGMSEACLHRKSH | 18 |
| Thyroid | SERRSTLKNEQSSPHLIQTTWTSSIFHLDHDDVNDQSVSSAQ | |
| Hormone | TFQTEEKKCKGYIPSYLDKDELCVVCGDKATGYHYRCITCEG | |
| Receptor Beta/ | CKGFFRRTIQKNLHPSYSCKYEGKCVIDKVTRNQCQECRFKK | |
| THRB, | CIYVGMATDLVLDDSKRLAKRKLIEENREKRRREELQKSIGH | |
| ERBA2, | KPEPTDEEWELIKTVTEAHVATNAQGSHWKQKRKFLPEDIGQ | |
| GRTH, PRTH/ | APIVNAPEGGKVDLEAFSHFTKIITPAITRVVDFAKKLPMFC | |
| P10828 | ELPCEDQIILLKGCCMEIMSLRAAVRYDPESETLTLNGEMAV | |
| | TRGQLKNGGLGVVSDAIFDLGMSLSSFNLDDTEVALLQAVLL | |
| | MSSDRPGLACVERIEKYQDSFLLAFEHYINYRKHHVTHFWPK | |
| | LLMKVTDLRMIGACHASRFLHMKVECPTELFPPLFLEVFED | |
| No. 19/ | MNAFLLSALCLLGAWAALAGGVTVQDGNFSFSLESVKKLKDL | 19 |
| Guanylate | QEPQEPRVGKLRNFAPIPGEPVVPILCSNPNFPEELKPLCKE | |
| Cyclase | PNAQEILQRLEEIAEDPGTCEICAYAACTGC | |
| Activator 2A/ | | |
| GUC2A, | | |
| GUCA2, | | |
| GUCA2A/ | | |
| Q02747 | | |
| No. 20/ | MTPLLTLILVVLMGLPLAQALDCHVCAYNGDNCFNPMRCPAM | 20 |
| Ly6/Neurotoxin | VAYCMTTRTSAAEAIWCHQCTGFGGCSHGSRCLRDSTHCVTT | |
| 1/LYNX1/ | ATRVLSNTEDLPLVTKMCHIGCPDIPSLGLGPYVSIACCQTS | |
| P0DP58 | LCNHD | |
| No. 21/ | MSEDSRGDSRAESAKDLEKQLRLRVCVLSELQKTERDYVGTL | 21 |
| Phospha- | EFLVSAFLHRMNQCAASKVDKNVTEETVKMLFSNIEDILAVH | |
| tidylinositol- | KEFLKVVEECLHPEPNAQQEVGTCFLHFKDKFRIYDEYCSNH | |
| 3,4,5- | EKAQKLLLELNKIRTIRTFLLNCMLLGGRKNTDVPLEGYLVT | |
| Trisphosphate | PIQRICKYPLILKELLKRTPRKHSDYAAVMEALQAMKAVCSN | |
| Dependent | INEAKRQMEKLEVLEEWQSHIEGWEGSNITDTCTEMLMCGVL | |
| Rac Exchange | LKISSGNIQERVFFLFDNLLVYCKRKHRRLKNSKASTDGHRY | |
| Factor 2/ | LFRGRINTEVMEVENVDDGTADFHSSGHIVVNGWKIHNTAKN | |
| PREX2, | KWFVCMAKTPEEKHEWFEAILKERERRKGLKLGMEQDTWVMI | |
| DEPDC2/ | SEQGEKLYKMMCRQGNLIKDRKRKLTTFPKCFLGSEFVSWLL | |
| Q70Z35 | EIGEIHRPEEGVHLGQALLENGIIHHVTDKHQFKPEQMLYRF | |
| | RYDDGTFYPRNEMQDVISKGVRLYCRLHSLFTPVIRDKDYHL | |
| | RTYKSVVMANKLIDWLIAQGDCRTREEAMIFGVGLCDNGFMH | |
| | HVLEKSEFKDEPLLFRFFSDEEMEGSNMKHRLMKHDLKVVEN | |
| | VIAKSLLIKSNEGSYGFGLEDKNKVPIIKLVEKGSNAEMAGM | |
| | EVGKKIFAINGDLVFMRPFNEVDCFLKSCLNSRKPLRVLVST | |
| | KPRETVKIPDSADGLGFQIRGFGPSVVHAVGRGTVAAAAGLH | |
| | PGQCIIKVNGINVSKETHASVIAHVTACRKYRRPTKQDSIQW | |
| | VYNSIESAQEDLQKSHSKPPGDEAGDAFDCKVEEVIDKFNTM | |
| | AIIDGKKEHVSLTVDNVHLEYGVVYEYDSTAGIKCNVVEKMI | |
| | EPKGFFSLTAKILEALAKSDEHFVQNCTSLNSLNEVIPTDLQ | |
| | SKFSALCSERIEHLCQRISSYKKFSRVLKNRAWPTFKQAKSK | |
| | ISPLHSSDFCPTNCHVNVMEVSYPKTSTSLGSAFGVQLDSRK | |
| | HNSHDKENKSSEQGKLSPMVYIQHTITTMAAPSGLSLGQQDG | |
| | HGLRYLLKEEDLETQDIYQKLLGKLQTALKEVEMCVCQIDDL | |
| | LSSITYSPKLERKTSEGIIPTDSDNEKGERNSKRVCFNVAGD | |
| | EQEDSGHDTISNRDSYSDCNSNRNSIASFTSICSSQCSSYFH | |
| | SDEMDSGDELPLSVRISHDKQDKIHSCLEHLFSQVDSITNLL | |
| | KGQAVVRAFDQTKYLTPGRGLQEFQQEMEPKLSCPKRLRLHI | |
| | KQDPWNLPSSVRTLAQNIRKFVEEVKCRLLLALLEYSDSETQ | |
| | LRRDMVFCQTLVATVCAFSEQLMAALNQMFDNSKENEMETWE | |
| | ASRRWLDQIANAGVLFHFQSLLSPNLTDEQAMLEDTLVALFD | |
| | LEKVSFYFKPSEEEPLVANVPLTYQAEGSRQALKVYFYIDSY | |
| | HFEQLPQRLKNGGGFKIHPVLFAQALESMEGYYYRDNVSVEE | |
| | FQAQINAASLEKVKQYNQKLRAFYLDKSNSPPNSTSKAAYVD | |
| | KLMRPLNALDELYRLVASFIRSKRTAACANTACSASGVGLLS | |
| | VSSELCNRLGACHIIMCSSGVHRCTLSVTLEQAIILARSHGL | |
| | PPRYIMQATDVMRKQGARVQNTAKNLGVRDRTPQSAPRLYKL | |
| | CEPPPPAGEE | |
| No. 22/ | MKWVWALLLLAALGSGRAERDCRVSSFRVKENFDKARFSGTW | 22 |
| Retinol | YAMAKKDPEGLFLQDNIVAEFSVDETGQMSATAKGRVRLLNN | |
| Binding | WDVCADMVGTFTDTEDPAKFKMKYWGVASFLQKGNDDHWIVD | |
| Protein 4/ | TDYDTYAVQYSCRLLNLDGTCADSYSFVFSRDPNGLPPEAQK | |
| RET4, RBP4/ | IVRQRQEELCLARQYRLIVHNGYCDGRSERNLL | |
| P02753 | | |
| No. 23 | Patient Age | |
| No. 24 | Patient Gender | |

Biomarkers contemplated herein also include polypeptides having an amino acid sequence identical to a listed marker of Table 1 over a span of 6 residues, 7 residues, 8 residues, 9, residues, 10 residues, 20 residues, 50 residues, or alternately 5%, 10%, 20%, 30%, 40%, 50%, 60%, 70% 80% 90%, 95% or greater than 95% of the sequence of the biomarker. Variant or alternative forms of the biomarker include for example polypeptides encoded by any splice-variants of transcripts encoding the disclosed biomarkers. In certain cases the modified forms, fragments, or their corresponding RNA or DNA, may exhibit better discriminatory power in diagnosis than the full-length protein.

Biomarkers contemplated herein also include truncated forms or polypeptide fragments of any of the proteins described herein. Truncated forms or polypeptide fragments of a protein can include N-terminally deleted or truncated forms and C-terminally deleted or truncated forms. Truncated forms or fragments of a protein can include fragments arising by any mechanism, such as, without limitation, by alternative translation, exo- and/or endo-proteolysis and/or degradation, for example, by physical, chemical and/or enzymatic proteolysis. Without limitation, a biomarker may comprise a truncated or fragment of a protein, polypeptide or peptide may represent about 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100% of the amino acid sequence of the protein.

Without limitation, a truncated or fragment of a protein may include a sequence of about 5-20 consecutive amino acids, or about 10-50 consecutive amino acids, or about 20-100 consecutive amino acids, or about 30-150 consecutive amino acids, or about 50-500 consecutive amino acid residues of the corresponding full length protein.

In some instances, a fragment is N-terminally and/or C-terminally truncated by between 1 and about 20 amino acids, such as, for example, by between 1 and about 15 amino acids, or by between 1 and about 10 amino acids, or by between 1 and about 5 amino acids, compared to the corresponding mature, full-length protein or its soluble or plasma circulating form.

Any protein biomarker of the present disclosure such as a peptide, polypeptide or protein and fragments thereof may also encompass modified forms of said marker, peptide, polypeptide or protein and fragments such as bearing post-expression modifications including but not limited to, modifications such as phosphorylation, glycosylation, lipidation, methylation, selenocystine modification, cysteinylation, sulphonation, glutathionylation, acetylation, oxidation of methionine to methionine sulphoxide or methionine sulphone, and the like.

In some instances, a fragmented protein is N-terminally and/or C-terminally truncated. Such fragmented protein can comprise one or more, or all transitional ions of the N-terminally (a, b, c-ion) and/or C-terminally (x, y, z-ion) truncated protein or peptide. Exemplary human markers, nucleic acids, proteins or polypeptides as taught herein are as annotated under NCBI Genbank (accessible at the website ncbi.nlm.nih.gov) or Swissprot/Uniprot (accessible at the website uniprot.org) accession numbers. In some instances said sequences are of precursors (for example, preproteins) of the of markers, nucleic acids, proteins or polypeptides as taught herein and may include parts which are processed away from mature molecules. In some instances although only one or more isoforms is disclosed, all isoforms of the sequences are intended.

Antibodies for the detection of the biomarkers listed herein are commercially available.

For a given biomarker panel recited herein, variant biomarker panels differing in one or more than one constituent are also contemplated. Thus, turning to a lead CRC panel A2GL, ALS, PTPRJ, and also including individual age, as an example, a number of related panels are disclosed. For this and other panels disclosed herein, variants are contemplated comprising at least 3, or at least 2 of the biomarker constituents of a recited biomarker panel.

Provided herein are methods that utilize biomarker panels to assess health status such as, for example, colorectal cancer health status. The methods can provide a high AUC signal that arises from a small pool of markers in the panel. In some cases, the AUC signal arises from no more than 20, 15, 10, 9, 8, 7, 6, 5, or 4 markers in the panel. The panel may include a list of markers from which a smaller subset of markers provide an AUC signal of at least 0.70, 0.75, 0.80, 0.81, 0.82, 0.83, 0.84, 0.85, 0.86, 0.87, 0.88, 0.89, 0.90, 0.91, 0.92, 0.93, 0.94, 0.95, 0.96, 0.97, 0.98, or 0.99. For example, a biomarker panel may comprise a panel of at least one marker selected from A2GL, ALS, and PTPRJ (and optionally age), and at least one additional marker such as one listed in Table 1. In some cases, the biomarker panel used to assess a colorectal health status comprises no more than 20, 15, 10, 9, 8, 7, 6, 5, or 4 markers. The biomarker panel may comprise markers selected from Table 1. In some cases, the biomarker panel consists of A2GL, ALS, PTPRJ, and age. In some cases, the biomarker panel consists essentially of A2GL, ALS, PTPRJ, and age. In some instances, the assessment of colorectal health status comprises utilizing a ratio between one or more of A2GL, ALS, and PTPRJ with age. For example, a classifier utilizing the biomarker panel to generate a prediction or classification (e.g., health status assessment) may utilize the ratio between PTPRJ and age as a feature in making the prediction. A biomarker panel comprising A2GL, ALS, PTPRJ, and age may include additional markers such as any combination of those listed in Table 1 or the list of 430 candidate markers described herein. In some cases, the biomarker panel comprises at least 1, 2, 3, 4, 5, 6, 7, 8, 9, 10, 11, 12, 13, 14, 15, 16, 17, 18, 19, 20, 21, 22, or at least 23 markers from Table 1. The biomarker panel can comprise any reference listed in Table 2 in combination with at least 1, 2, 3, 4, 5, 6, 7, 8, 9, 10, 11, 12, 13, 14, 15, 16, 17, 18, 19, or at least 20 additional markers (e.g., non-redundant markers) from Table 1. In some instances, the biomarker panel comprises at least 1, 2, 3, 4, or 6 of A2GL, ALS, PTPRJ, GELS, and TFRC1. An exemplary panel comprises A2GL, ACTBM, ALS, APOC4, APOE, APOL1, CHLE, IL10R, ITIH2, KAIN, PON1, PTPRJ, SPP24, TFR1, and TNF15. In some instances, a biomarker panel comprises at least 1, 2, 3, 4, 5, 6, 7, 8, 9, 10, 11, 12, 13, or 14 proteins selected from A2GL, ACTBM, ALS, APOC4, APOE, APOL1, CHLE, IL10R, ITIH2, KAIN, PON1, PTPRJ, SPP24, TFR1, TNF15, and optionally including age. Another exemplary panel comprises A2GL, ALS, PTPRJ, GELS, and TFR1. Sometimes, a biomarker panel comprises at least 1, 2, 3, or 4 of A2GL, ALS, PTPRJ, GELS, and TFR1, alone or in combination with age. The biomarker panel can comprise a ratio of a biomarker and age such as, for example, PTPRJ/age.

Exemplary CRC panels consistent with the disclosure herein are listed in Table 2. Also disclosed are panels comprising the markers listed in entries of Table 2.

TABLE 2
CRC biomarker panel constituents

| Reference | CRC Protein Biomarker | Demographics | Features |
|---|---|---|---|
| 1 | A2GL, ALS, PTPRJ | None | 3 |
| 2 | A2GL, ALS | None | 2 |
| 3 | A2GL, PTPRJ | None | 2 |
| 4 | ALS, PTPRJ | None | 2 |
| 5 | A2GL | None | 1 |
| 6 | ALS | None | 1 |
| 7 | PTPRJ | None | 1 |
| 8 | A2GL, ALS, PTPRJ | Age | 4 |
| 9 | A2GL, ALS | Age | 3 |
| 10 | A2GL, PTPRJ | Age | 3 |
| 11 | ALS, PTPRJ | Age | 3 |
| 12 | A2GL | Age | 2 |
| 13 | ALS | Age | 2 |
| 14 | PTPRJ | Age | 2 |

In some cases, the panel comprises reference 1 of Table 2 in combination with at least one additional marker from Table 1. In some cases, the panel comprises reference 2 of Table 2 in combination with at least one additional marker from Table 1. In some cases, the panel comprises reference 3 of Table 2 in combination with at least one additional marker from Table 1. In some cases, the panel comprises reference 4 of Table 2 in combination with at least one additional marker from Table 1. In some cases, the panel comprises reference 5 of Table 2 in combination with at least one additional marker from Table 1. In some cases, the panel comprises reference 6 of Table 2 in combination with at least one additional marker from Table 1. In some cases, the panel comprises reference 7 of Table 2 in combination with at least one additional marker from Table 1. In some cases, the panel comprises reference 8 of Table 2 in combination with at least one additional marker from Table 1. In some cases, the panel comprises reference 9 of Table 2 in combination with at least one additional marker from Table 1. In some cases, the panel comprises reference 10 of Table 2 in combination with at least one additional marker from Table 1. In some cases, the panel comprises reference 11 of Table 2 in combination with at least one additional marker from Table 1. In some cases, the panel comprises reference 12 of Table 2 in combination with at least one additional marker from Table 1. In some cases, the panel comprises reference 13 of Table 2 in combination with at least one additional marker from Table 1. In some cases, the panel comprises reference 14 of Table 2 in combination with at least one additional marker from Table 1. In some cases, the biomarker panel comprises any reference of Table 2 in combination with GELS from Table 1. In some cases, the biomarker panel comprises any reference of Table 2 in combination with TFR1 from Table 1.

### Proteomics and Other Affinity Assay Workflows

The present disclosure includes methods that address various shortcomings with a targeted proteomics workflow that enable Tier 2 measurements of targeted peptides using mass spectrometry. In some instances, the measurements are obtained using dynamic multiple reaction monitoring (dMRM) MS. Described herein are various steps taken, including process controls, to develop and characterize a mass spectrometric analysis such as, for example, a high-multipex dMRM assay. Alternative assays are also consistent with the disclosure herein. For example, affinity assays using antibodies or antibody mimetics such as affibody molecules, affitins, atrimers, etc., may be used to detect and/or quantify markers. Affinity assays can include immunoassays and aptamer assays. In some cases, the assay measures proteotypic peptides from proteins related to a disease or health status. For example, described herein are assays measuring 641 proteotypic peptides from 392 colorectal cancer (CRC) related proteins. The present disclosure includes the use of quality and/or process control metrics and procedures to track and handle sample processing and instrument variations over a data collection period (e.g., of four months), during which the assay was used in the study of biological samples from patients with CRC symptoms. The biological samples can be obtained from various sources such as, for example, blood samples. The samples for 1,045 patients with CRC symptoms were analyzed in one study. After data collection, transitions can be filtered using one or more signal quality metrics before being used in receiver operating characteristic (ROC) analysis to assess univariate CRC signal. As an example, the ROC analysis demonstrated dMRM-based CRC signal carried by 127 CRC-related proteins in the symptomatic population. These dMRM assays can be developed as Tier 1 assays for clinical tests to identify individuals at elevated risk of CRC.

In some cases, transitions are filtered using at least one, at least two, at least three, at least four, at least five, at least six, at least seven, at least eight, at least nine, or at least ten signal quality metrics before being used in ROC analysis for assessing univariate CRC signal.

Disclosed herein is a dMRM MS method with the rigor of a Tier 2 assay as defined by the CPTAC ‘fit for purpose approach’. Using quality and process control procedures, the assay was successfully used to quantify 641 proteotypic peptides representing 392 CRC-related proteins in plasma from 1045 CRC-symptomatic patients. The results showed that 127 of the proteins carried univariate CRC signal in the symptomatic population. This large number of single biomarkers demonstrates the utility of multivariate classifiers to distinguish CRC in the symptomatic population using the disclosed workflow(s). Other methodologies in addition to dMRM MS may be used. Immunoassays and aptamer assays that utilize antibodies, aptamers, or other molecules capable of binding or recognizing specific targets are consistent with the methods and workflows described herein.

Various forms of mass spectrometry are available for evaluating protein and other molecules in a sample. For example, fragmenting approaches for tandem MS include collision-induced dissociation (CID), electron capture dissociation (ECD), electron transfer dissociation (ETD), infrared multiphoton dissociation (IRMPD), blackbody infrared radiative dissociation (BIRD), electron-detachment dissociation (EDD) and surface-induced dissociation (SID). Various separation techniques are available as well and include, for example, gas chromatography, liquid chromatography, and capillary electrophoresis.

Disclosed herein are quality and process control procedures that allow the generation of biomarker panels for assessing colorectal health status. Such procedures include process control and/or quality control steps for evaluating performance of the assays and/or instruments used to process samples. A process control step can include system suitability tests (SST) that are performed prior to sample processing. For example, SSTs can be performed on mass spectrometry instrumentation to evaluate performance of the liquid chromatography and/or mass spectrometer. Control samples can be used in this evaluation such as, for example, to generate standard curves of internal standards to assess the instrumentation and workflow. An example of a process control step is to determine whether 10× dilution series of internal standards are being accurately quantified by the mass spectrometer (or other affinity assay such as immunoassay or aptamer assay). The process control step may also determine whether the dynamic range spans across a threshold number of log units across the standard curve. For example, a lack of accuracy in quantification and/or a low dynamic range can cause the sample to be discarded and/or gated/screened to remove data determined to be impacted by the areas of poor performance. A process control step that evaluates at least one QC marker is also consistent with the present disclosure. In some cases, a control sample includes at least one QC marker as described herein.

Process control steps can include various forms of workflow monitoring such as, for example, monitoring flow-through AUC during immunodepletion, monitoring of TPA results for sample processing and immunodepletion efficiency, or sample preparation customization depending on the TPA result of each individual sample. Other examples of process control steps include a quality control check requiring a confidence interval of RTs of heavy transitions to be no more than a certain percentage from the margins of a chromatography mass spectrometry acquisition window. Examples of the certain percentage include 1%, 2%, 3%, 4%, 5%, 6%, 7%, 8%, 9%, 10%, 15%, and 20%. Workflow monitoring utilizing QC markers to assess various conditions such as sample integrity, sample elution efficiency, sample storage condition, and internal standard monitoring are also contemplated in the present disclosure.

Biomarkers or biological markers can refer to any measurable characteristic of a biological specimen that can be evaluated as an indicator of normal biological processes, pathogenic processes or pharmacological responses to a therapeutic intervention. In the last 30 years, a greater understanding of the underlying biology of many cancers coupled with technological advances have contributed to the investment in biomarker discovery with the hope of identifying the appropriate biological markers to guide clinicians in the detection, screening, diagnosis, treatment and monitoring of cancer treatment. Among the plethora of biomarker-related publications of recent years there have been numerous reports on the discovery and promise of novel plasma- or serum-based cancer biomarkers, intended for diagnostic, prognostic and predictive purposes. However, despite the abundance of biomarker publications and the advances in genomic and proteomic technologies, few biomarkers have been implemented in clinical practice; by some estimates the success rate for clinical translation of biomarkers is as low as 0.1%, with only a few dozen biomarkers in clinical use for the treatment of cancer. While some have speculated on the factors contributing to the failures of biomarkers reaching the clinic, it is widely recognized that a large number of these failures can be categorized as false discoveries—biomarkers that could not be independently reproduced in follow-up studies.

The present disclosure recognizes that these false discoveries can be attributed to pre-analytical, analytical, and post-analytical shortcomings. The pre-analytical problems may stem from poor sample quality and/or incomplete clinical documentation. The analytical problems may originate from varying qualities of assay platforms and sample measurements. The post-analytical problems may result from faulty bioinformatics approaches (statistical problems related to multiple testing and overfitting). In light of the poor return on investment in biomarker discovery, in recent years, the scientific community has started to focus on identifying and addressing these issues contributing to high biomarker failure rate.

In some instances, analytical variation and address factors contributing to false biomarker discovery are monitored. These are particularly troublesome in multiplexed biomarker studies, where the variabilities of several assays must be tracked and managed to ensure success. The multi-marker assay presented in this manuscript can be classified as a Tier 2 assay under the CPTAC ‘fit for purpose approach’; it was developed to measure colorectal cancer candidate biomarker proteins with the goal of down-selecting to a much smaller protein panel, for further validation and eventual clinical implementation. A Tier 2 assay should be high-throughput, precise, reproducible and quantitative and it's because of these requirements as well as it's multiplexing capabilities that targeted dMRM was selected in this study with the goal of identifying a novel colorectal biomarker panel. While selecting the best technology platform for clinical utility will no doubt improve the odds of successful delivery of a clinical biomarker, it is also important to address the variability associated with the highly complex analytical process. To this end, an important consideration is the implementation of system suitability tests (SST) and quality controls to aid in monitoring and remedying the variability. Recent publications also support the growing recognition of the need for SST and quality controls as a means to addressing analytical variability and establishing confidence in analytical measurements.

Described herein is the development and experimental steps of a large-scale dMRM-based method for identifying biomarkers relevant to disease or health status. In some cases, the method implements SST, using SIS peptide mixture and pooled plasma sample as reference material, to evaluate aspects of the analytical instrumentation such as consistency of the response, carryover, retention time stability, and signal-to-noise. In certain instances, quality controls are used in the form of pooled plasma sample to monitor and if needed, correct the analytical variability during sample processing and analysis. The implementation of one or more systematic quality assessments was a critical component of the analytical process, providing confidence in over a thousand samples measurements, collected on multiple instruments over an extended period of time.

Described herein are systems and methods that address the analytical variability, and the pre-analytical factors impacting sample quality, were also an important consideration in the study design. The samples used in this study were from the same carefully curated cohort as used in previous biomarker studies and described in more detail in an earlier publication. In addition to the measures taken to monitor analytical variability in this report, described herein is a novel systematic approach used to filter peptides and rank peptide transitions, as a means to build a robust mass spectrometry analytical method such as, for example, a dMRM-based analytical method, for the measurement of proteotypic peptides representing disease or health condition related proteins. For example, disclosed herein are measurements of 641 proteotypic peptides representing 392 CRC-related proteins. Finally, with a dataset of reliable analytical measurements from various patients and under the guidance of a team of bioinformatics scientists, machine learning algorithms were used to analyze the quantitative measurements and to build candidate CRC biomarker panels suitable for identifying at-risk patients who should undergo colonoscopy. Described herein are biomarker panels generated based on measurements and analysis of 1045 CRC patients.

### Candidate Biomarkers

Candidate protein biomarkers for CRC can be selected from various sources such as one or more of: 1) an earlier targeted proteomics study performed in our laboratory, 2) analysis of publicly available proteomics datasets related to CRC, and 3) semi-automated literature searches. A non-limiting list of candidate protein biomarkers identified is shown below, which has a total of 430 proteins designated as CRC-related biomarker candidates for further experimental investigation.

1433B_HUMAN; CH60_HUMAN; H2BFS_HUMAN; PCKGM_HUMAN; TNF15_HUMAN; 1433E_HUMAN; CHK1_HUMAN; HABP2_HUMAN; PDIA3_HUMAN; TNF6B_HUMAN; 1433F_HUMAN; CHK2_HUMAN; HEMO_HUMAN; PDIA6_HUMAN; TP4A3_HUMAN; 1433G_HUMAN; CHLE_HUMAN; HEP2_HUMAN; PDLI7_HUMAN; TPA_HUMAN; 1433T_HUMAN; CLC4D_HUMAN; HGF_HUMAN; PDXK_HUMAN; TPM2_HUMAN; 1433Z_HUMAN; CLUS_HUMAN; HMGB1_HUMAN; PEBP1_HUMAN; TR10B_HUMAN; 1A68_HUMAN; CNDP1_HUMAN; HNRPF_HUMAN; PEDF_HUMAN; TRAP1_HUMAN; A1AG1_HUMAN; CNN1_HUMAN; HNRPQ_HUMAN; PGFRA_HUMAN; TREM1_HUMAN; A1AG2_HUMAN; CO3_HUMAN; HPT_HUMAN; PIPNA_HUMAN; TRFE_HUMAN; A1AT_HUMAN; CO4A_HUMAN; HRG_HUMAN; PLGF_HUMAN; TRFL_HUMAN; A1BG_HUMAN; CO6A3_HUMAN; HS90B_HUMAN; PLIN2_HUMAN; TRI33_HUMAN; A2AP_HUMAN; CO8G_HUMAN; HSPB1_HUMAN; PLMN_HUMAN; TSG6_HUMAN; A2GL_HUMAN; CO9_HUMAN; I10R1_HUMAN; PO2F1_HUMAN; TSP1_HUMAN; A2MG_HUMAN; COR1C_HUMAN; IBP2_HUMAN; PON1_HUMAN; TTHY_HUMAN; A4_HUMAN; CORIN_HUMAN; IBP3_HUMAN; POTEF_HUMAN; UGDH_HUMAN; AACT_HUMAN; CP1A1_HUMAN; IF4A3_HUMAN; PPIB_HUMAN; UGPA_HUMAN; ABCB5_HUMAN; CRDL2_HUMAN; IFT74_HUMAN; PRD16_HUMAN; UROK_HUMAN; ABCBA_HUMAN; CRP_HUMAN; IGF1_HUMAN; PRDX1_HUMAN; VCAM1_HUMAN; ACINU_HUMAN; CSF1_HUMAN; IGHA2_HUMAN; PRDX2_HUMAN; VEGFA_HUMAN; ACTBL_HUMAN; CSF1R_HUMAN; IGLL5_HUMAN; PREX2_HUMAN; VGFR1_HUMAN; ACTBM_HUMAN; CSPG2_HUMAN; IKKB_HUMAN; PRKN2_HUMAN; VILI_HUMAN; ACTG_HUMAN; CTHR1_HUMAN; IL23R_HUMAN; PRL_HUMAN; VIME_HUMAN; ACTH_HUMAN; CTNA1_HUMAN; IL26_HUMAN; PROC_HUMAN; VNN1_HUMAN; ADIPO_HUMAN; CTNB1_HUMAN; IL2RB_HUMAN; PROS_HUMAN; VP13B_HUMAN; ADT2_HUMAN; CUL1_HUMAN; IL6RA_HUMAN; PSME3_HUMAN; VTNC_HUMAN; AFAM_HUMAN; CYTC_HUMAN; IL8_HUMAN; PTEN_HUMAN; VWF_HUMAN; AGAP2_HUMAN; DAF_HUMAN; IL9_HUMAN; PTGDS_HUMAN; XBP1_HUMAN; AKA12_HUMAN; DEF1_HUMAN; ILEU_HUMAN; PTPRJ_HUMAN; ZA2G_HUMAN; AKT1_HUMAN; DESM_HUMAN; IPSP_HUMAN; PTPRT_HUMAN; ZMIZ1_HUMAN; AL1A1_HUMAN; DHRS2_HUMAN; IPYR_HUMAN; PTPRU_HUMAN; ZPI_HUMAN; AL1B1_HUMAN; DHSA_HUMAN; IRGM_HUMAN; PZP_HUMAN; ALBU_HUMAN; DPP10_HUMAN; ISK1_HUMAN; RAB38_HUMAN; ALDOA_HUMAN; DPP4_HUMAN; ITA6_HUMAN; RASF2_HUMAN; ALDR_HUMAN; DPYL2_HUMAN; ITA9_HUMAN; RASK_HUMAN; ALS_HUMAN; DYHC1_HUMAN; ITIH2_HUMAN; RBX1_HUMAN; AMPD1_HUMAN; ECH1_HUMAN; JAM3_HUMAN; RCAS1_HUMAN; AMPN_HUMAN; EDA_HUMAN; K1C19_HUMAN; REG4_HUMAN; AMY2B_HUMAN; EF2_HUMAN; K2C72_HUMAN; RET4_HUMAN; ANGI_HUMAN; ENOA_HUMAN; K2C73_HUMAN; RHOA_HUMAN; ANGL4_HUMAN; ENOX2_HUMAN; K2C8_HUMAN; RHOB_HUMAN; ANGT_HUMAN; ENPL_HUMAN; KAIN_HUMAN; RHOC_HUMAN; ANT3_HUMAN; ENPP1_HUMAN; KC1D_HUMAN; ROA1_HUMAN; ANXA1_HUMAN; ENPP2_HUMAN; KCRB_HUMAN; ROA2_HUMAN; ANXA3_HUMAN; EZRI_HUMAN; KISS1_HUMAN; RRBP1_HUMAN; ANXA4_HUMAN; FA10_HUMAN; KLK6_HUMAN; RSSA_HUMAN; ANXA5_HUMAN; FA5_HUMAN; KLOT_HUMAN; S100P_HUMAN; APC_HUMAN; FA7_HUMAN; KNG1_HUMAN; S10A8_HUMAN; APCD1_HUMAN; FA9_HUMAN; KPCD1_HUMAN; S10A9_HUMAN; APOA1_HUMAN; FABP5_HUMAN; KPYM_HUMAN; S10AB_HUMAN; APOA2_HUMAN; FAK1_HUMAN; LAMA2_HUMAN; S10AC_HUMAN; APOA4_HUMAN; FAK2_HUMAN; LAT1_HUMAN; S29A1_HUMAN; APOA5_HUMAN; FARP1_HUMAN; LBP_HUMAN; SAA1_HUMAN; APOC1_HUMAN; FBX4_HUMAN; LCAT_HUMAN; SAA2_HUMAN; APOC4_HUMAN; FCGBP_HUMAN; LDHA_HUMAN; SAA4_HUMAN; APOE_HUMAN; FCRL3_HUMAN; LEG2_HUMAN; SAHH_HUMAN; APOH_HUMAN; FCRL5_HUMAN; LEG3_HUMAN; SAMP_HUMAN; APOL1_HUMAN; FETA_HUMAN; LEG4_HUMAN; SBP1_HUMAN; APOM_HUMAN; FETUA_HUMAN; LEG8_HUMAN; SDCG3_HUMAN; ASAP3_HUMAN; FHL1_HUMAN; LEPR_HUMAN; SEGN_HUMAN; ATPB_HUMAN; FHR1_HUMAN; LEUK_HUMAN; SELPL_HUMAN; ATS13_HUMAN; FHR3_HUMAN; LG3BP_HUMAN; SEPP1_HUMAN; B2CL1_HUMAN; FIBA_HUMAN; LMNB1_HUMAN; SEPR_HUMAN; B2LA1_HUMAN; FIBB_HUMAN; LRRC7_HUMAN; SEPT9_HUMAN; B3GT5_HUMAN; FIBG_HUMAN; LUM_HUMAN; SF3B3_HUMAN; BANK1_HUMAN; FINC_HUMAN; LYNX1_HUMAN; SHIP1_HUMAN; BC11A_HUMAN; FLNA_HUMAN; LYSC_HUMAN; SHRPN_HUMAN; BCAR1_HUMAN; FLNB_HUMAN; MACF1_HUMAN; SIA8D_HUMAN; C1QBP_HUMAN; FLNC_HUMAN; MAP1S_HUMAN; SIAL_HUMAN; C4BPA_HUMAN; FND3B_HUMAN; MARE1_HUMAN; SIT1_HUMAN; CA195_HUMAN; FRIH_HUMAN; MASP1_HUMAN; SKP1_HUMAN; CAH1_HUMAN; FRIL_HUMAN; MASP2_HUMAN; SLAF1_HUMAN; CAH2_HUMAN; FRMD3_HUMAN; MBL2_HUMAN; SO1B3_HUMAN; CALR_HUMAN; FST_HUMAN; MCM4_HUMAN; SP110_HUMAN; CAPG_HUMAN; FUCO_HUMAN; MCR_HUMAN; SPB6_HUMAN; CASP9_HUMAN; FUCO2_HUMAN; MCRS1_HUMAN; SPON2_HUMAN; CATD_HUMAN; G3P_HUMAN; MIC1_HUMAN; SPP24_HUMAN; CATS_HUMAN; GAS6_HUMAN; MICA1_HUMAN; SRC_HUMAN; CATZ_HUMAN; GBRA1_HUMAN; MIF_HUMAN; SRPX2_HUMAN; CBG_HUMAN; GDF15_HUMAN; MMP2_HUMAN; STK11_HUMAN; CBPN_HUMAN; GDIR1_HUMAN; MMP7_HUMAN; SYDC_HUMAN; CBPQ_HUMAN; GELS_HUMAN; MMP9_HUMAN; SYG_HUMAN; CCD83_HUMAN; GFI1B_HUMAN; MTG16_HUMAN; SYNE1_HUMAN; CCL14_HUMAN; GGT1_HUMAN; MUC24_HUMAN; SYUG_HUMAN; CCR5_HUMAN; GHRL_HUMAN; MYL6_HUMAN; TACC1_HUMAN; CD109_HUMAN; GPNMB_HUMAN; MYL9_HUMAN; TAL1_HUMAN; CD20_HUMAN; GPX3_HUMAN; MYO9B_HUMAN; TBB1_HUMAN; CD24_HUMAN; GREM1_HUMAN; NDKA_HUMAN; TCTP_HUMAN; CD248_HUMAN; GRM6_HUMAN; NDRG1_HUMAN; TETN_HUMAN; CD28_HUMAN; GRP75_HUMAN; NFAC1_HUMAN; TF7L1_HUMAN; CD63_HUMAN; GSHR_HUMAN; NGAL_HUMAN; TFR1_HUMAN; CDD_HUMAN; GSTP1_HUMAN; NIBL2_HUMAN; THBG_HUMAN; CEA_HUMAN; GUC2A_HUMAN; NIPBL_HUMAN; THIO_HUMAN; CEAM3_HUMAN; H13_HUMAN; NNMT_HUMAN; THRB_HUMAN; CEAM5_HUMAN; H2A1D_HUMAN; NOD2_HUMAN; THTR_HUMAN; CEAM6_HUMAN; H2A2B_HUMAN; NUPR1_HUMAN; TIE2_HUMAN; CERU_HUMAN; H2AX_HUMAN; OSTP_HUMAN; TIMP1_HUMAN; CFAH_HUMAN; H2B1A_HUMAN; P53_HUMAN; TIMP2_HUMAN; CFAI_HUMAN; H2B1L_HUMAN; PAFA_HUMAN; TKT_HUMAN; CGHB_HUMAN; H2B1O_HUMAN; PAI1_HUMAN; TMG4_HUMAN; CH3L1_HUMAN; H2B3B_HUMAN; PALLD_HUMAN; TNF13_HUMAN;

Described herein is are methods for carrying out CRC biomarker discovery using targeted MS measures obtained with dMRM assays. The present methods addressed a significant problem that has plagued MS-based biomarker discovery over the past few decades—that few discovery results translate successfully to the clinic. To ensure a better success rate in translating the results to the clinic, a large amount of work went toward developing dMRM assays of very high quality.

The methods described herein allowed the development of Tier 2 assays as defined by the CPTAC ‘fit for purpose approach’. In some cases, a number of process and quality controls were utilized throughout assay development, study running, and study analysis; some of these control steps included novel approaches. During assay development, process control steps were implemented in early in silico peptide filtering, LC gradient optimization, transition filtering, CE optimization, and transition screening/ranking for the final method build. The transition screening/ranking process used an automated approach that is novel in the field, and that offers several advantages to manual methods. During study runs, process control steps were implemented in monitoring of flow-through AUC during immunodepletion, monitoring of TPA results for sample processing and immunodepletion efficiency, and sample preparation customization depending on each sample's TPA result. During study runs, quality control steps were implemented in SSTs run to check LC and MS performance prior to each day's planned sample runs, and in tracking PQCs' signal and reproducibility across study days. During study analysis, transitions were filtered to those with quantitative performance and with good peak quality, thus ensuring that only the best measures entered into study analysis. The peak quality tool that we employed is novel in the field; its high performance enables quick assessment of peak quality and obviates requirement for lengthy manual peak review. In addition, we used only transitions that had valid measures across all study samples, thus avoiding the problems that accompany data imputation for missing values.

The study presented here resulted in evidence for CRC signal carried individually by 127 CRC-related proteins in the CRC-symptomatic population. This large number of CRC biomarkers in the symptomatic population, combined with the very high quality assays with which they were identified, demonstrates the potential for development of new CRC diagnostic tests serving the CRC-symptomatic population using our workflow.

### Classifiers for Assessing Health Status

The present disclosure describes work related to classifier builds performed as part of the project known as Targeted Proteomics Version 2 (TPv2). The classifiers were aimed at discriminating colorectal cancer (CRC) from non-CRC samples, using data from 1,045 Endoscopy II (CRC-symptomatic) patients' plasma samples. In TPv2, the sample concentrations of targeted peptide ions were obtained using a dynamic multiple-reaction-monitoring (MRM) method on mass spectrometry (MS) instruments (You et al., 2018). The initial goals of the work reported here were to develop CRC classifiers that 1) demonstrate an improvement of CRC signal over that reported in TPv1 (Jones et al., 2016) and/or 2) demonstrate CRC performance at least equivalent to that found in the SimpliProColon Version 1 CRC (SPCv1) test, which was developed based on ELISA measures from the same 1,045 Endoscopy II patients used in the present study. The first goal was determined to be unrealistic because of differences between the datasets used in TPv1 and TPv2. The second goal was met.

### Overview of the 58 Simple Grids

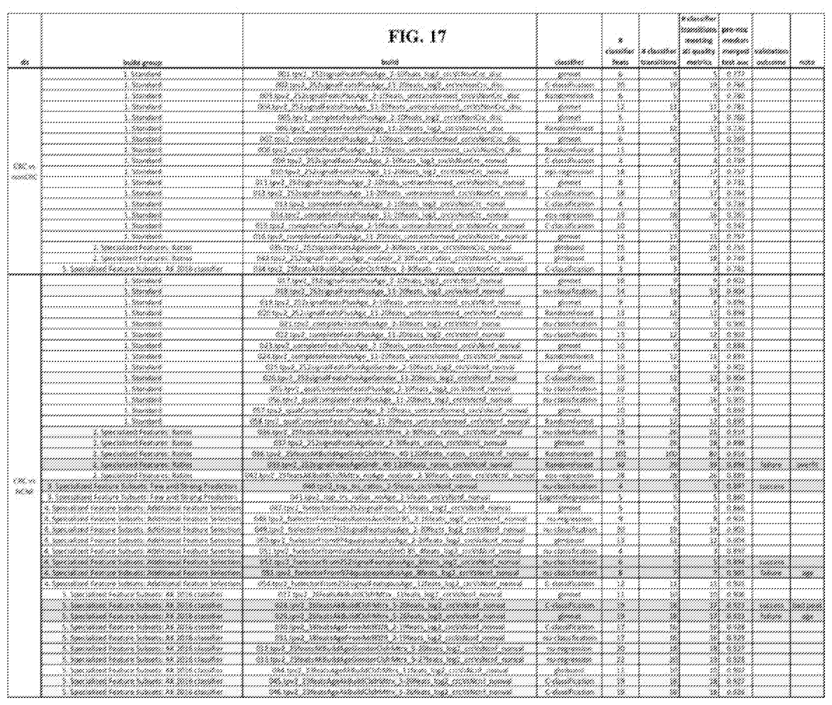

An overview of the 58 simple grids is presented in FIG. 17. The table is ordered first by discrimination tested (dx: CRC vs nonCRC, or CRC vs NCNF), then by build group, then by build number. Additional columns from left to right include classifier, number of classifier features, number of classifier transitions, number of classifier transitions meeting all quality metrics, pre-noc (‘pre-no call’) median merged test AUC, validation outcome, and notes. This table can be used as a guide to understanding the development and outcomes of the 58 classifier grids. The build groups include: standard, specialized features (e.g., including ratios), and earlier classifiers (e.g., AK 2016 classifier). The classifiers include: glmnet, C-classification, nu-classification, random forest, eps-regression, nu-regression, and glmboost. The number of classifier features range from 3 to 102. The number of classifier transitions range from 3 to 100. The number of classifier transitions that meet all quality metrics range from 3 to 80. The pre-noc median merged test AUCs range from 0.730 to 0.929. The validation outcomes showing selected successful and failed classifiers are indicated by shaded rows (4 shaded rows total). The top shaded row is a failure and has 40 features (notes indicate it was overfit) using a random forest classifier. The second top shaded row is a success with 4 features and 3 transitions with a 0.897 AUC using a nu-classification classifier. The third shaded row from the top is a success with 6 features, 5 transitions, and 0.894 AUC using a nu-classification classifier. The fifth shaded row from the top is a success with 19 features, 18 transitions, and 0.923 AUC using a c-classification classifier. The fourth and sixth shaded rows from the top were failures.

The column “pre-noc median merged test auc” lists the discovery set CRC vs NCNF AUCs achieved in each grid, prior to any NoC analyses. Considering just these AUCs, it's clear that the lowest AUCs were obtained for the CRC vs nonCRC discrimination, performed early in the process. This is consistent with other API studies using the same patient samples (CRC05E, which gave rise to the SPCv1 test). Based on this, the majority of later builds focused on the CRC vs NCNF discrimination. The highest AUCs were obtained for the CRC vs NCNF grids using the “AK 2016 classifier” feature subset. While AK's expanded grid often gave good classifiers in the past, this finding of highest AUCs was not entirely expected—only a subset of the AK 2016 classifier features was found in the data matrices that AK distributed to the team, and the peak areas appear to have been calculated using different algorithms than used by AK for his 2016 builds. Despite these differences, the highest AUCs were uncovered with these classifiers; this is another argument in favor of either recasting the simple grid with additional feature selection capabilities, or rehydrating the expanded grid,

Rows for classifiers for which NoC analyses were performed are highlighted in blue and orange in FIG. 17. In the earlier of the 58 grids, NoC analyses were applied generally, with some exceptions, to classifiers with AUCs near and above 0.91. As the grids proceeded, three patterns became clear and influenced later selection of classifiers for NoC analyses. The first pattern was that despite good AUCs and good NoC performance for classifiers based on AK 2016 classifier features, there was a large decrement in performance for these models in validation (models 28 and 29); technically model 28 validated, but sens and spec were below the SPCv1 sens and spec of 0.81/0.78. The second pattern was a tendency towards overfitting in classifiers with more features. This was tested explicitly in model 39, which had very strong NoC performance but failed validation because of statistically lower performance than observed in NoC′d discovery. The third pattern was that some ratios had very strong univariate performance.

These observations led to a revised approach focusing on using specialized feature subsets, and using fewer features. This eventually led to model 40, which validated with sens/spec matching that of SPCv1. The other notable success using this approach was model 52.

Comparison with TPv1

One of the initial goals of the work described here was to compare TPv2 results to those of TPv1 (Jones et al., 2016). The TPv1 study examined CRC vs non-CRC signal using samples from age- and gender-matched patient pairs in discovery and validation sets of 138 and 136 patients respectively. The patients came from three different cohorts that varied in control group composition and in information provided regarding comorbidities. At least one of the cohorts had a control group approximately equivalent to TPv2's NCNF (healthiest controls) group. TPv1 generated a 15-transition classifier with a discovery AUC of 0.82, and validated with an AUC of 0.91 and sens/spec of 0.87/0.81; this was higher than TPv2's validation AUC of 0.82 and sens/spec 0.81/0.78 for model 40.

There are several notable differences between TPv1 and TPv2, making a direct comparison challenging. Whereas TPv1 used matched samples and excluded demographic factors as CRC predictors, TPv1 randomized sample distribution and allowed age and gender to contribute to classifiers. Whereas TPv1 used three patient cohorts with varying annotation quality about comorbidities and symptomology, TPv2 used a single patient cohort with high quality annotations regarding comorbidities and symptomology. Whereas TPv1 samples may have had site bias correlated with CRC status for some cohorts, TPv2 samples were shown to have no site bias. Whereas TPv1 used a non-CRC group biased toward (and possibly dominated by) healthiest controls, TPv2 final classifiers used a non-CRC group representing the range of comorbidities in the actual ITT population. Whereas TPv1 did not use any information about patient CRC symptomology, TPv2 used only patients with CRC symptomology.

Of these differences, two can explain the larger CRC signal reported for the final TPv1 classifier: 1) bias toward healthy controls for the non-CRC group in TPv1, 2) potential site bias correlated with CRC status in TPv1. The first suggests that a more responsible comparison might be between TPv1 signal and TPv2's CRC vs NCNF signal. Considering TPv2's CRC vs NCNF discovery classifiers (Table 4) reveals that model 31 had a pre-NoC discovery AUC of 0.929, which is higher than the TPv1 discovery AUC of 0.81 at the same stage; taking model 31 forward into validation, and using the just the CRC vs NCNF subset there, might serve as an acceptable comparison with TPv1. This might be considered for future work, if a comparison with TPv1 is pursued further.

Comparison with SPCv1.

The second initial goal of the work described here was to demonstrate CRC performance at least equivalent to that found for the SPCv1 CRC test. The CRC05E study that gave rise to the SPCv1 test used samples from exactly the same patients as used in the current TPv2 study, with the same patients assigned to the discovery and validation sets. In addition, the SPCv1 classifier builds used the same approach as that used here—discovery CRC vs NCNF classifier builds, followed by NoC analyses in discovery ITT samples, followed by validation. Thus the results are directly comparable between the two studies. SPCv1 had a validated CRC vs non-CRC AUC of 0.83 and sens/spec of 0.81/0.78; TPv2 model 40 had a validated AUC of 0.82 (statistically indistinguishable from that of SPCv1) and sens/spec of 0.81/0.78; thus the TPv2 study demonstrated performance equivalent to that of SPCv1, meeting the goal.

The TPv2 classifier offers two advantages over that used in the SPCv1 test. First, the assay format, using targeted MRM MS measures, may prove to be more amenable to successful quality control and automation than the SPCv1 ELISAs. Second the smaller number of features in two of the best TPv2 classifiers (3 and 5 unique transition in models 40 and 52 respectively) will likely improve the focus and quality of any new test based on these results.

The work described here resulted in three validated CRC vs non-CRC classifiers targeted toward the CRC-symptomatic population. These classifiers were all SVMs, and arose from builds 28, 40, and 52. The classifier from build 40 is the most promising as it uses the fewest predictors and has the strongest performance in validation, matching sens/spec of 0.81/0.78 used in the SPCv1 test. This test, if implemented commercially on a MS platform, would provide equivalent CRC performance to SPCv1, and would likely prove more amenable to automation and quality control.

### Health Status Assessment

Disclosed herein are methods, systems, databases and compositions related to targeted health status assessment. Practice of the disclosure herein allows monitoring of a patient's health status, for example through the accurate, repeatable measurement of biomarkers such as proteins in an in vitro sample (e.g., derived from a patient). Monitoring may be directed toward a particular health status or condition, a set of conditions, or may be untargeted such that biomarkers are monitored and a change in biomarker levels or other signal from the biomarkers signals that a health condition indicated by the biomarkers or related to the biomarkers has changed or warrants further investigation or intervention.

Disclosed herein is a demonstration of the utility of mass spectrometry for the identification and quantitation of endogenous proteins and peptides in biological samples obtained from a human. Non-limiting examples of biological samples include dried blood or plasma spots, which can be collected using various collection methods such as special filter paper or dried plasma spot cards. In some embodiments of dried plasma spot cards, a blood sample is deposited on a filter layer that separates out the non-plasma blood components. After a specified amount of time, this filter layer is removed leaving a spot of plasma which is then left to dry prior to storage.

Biomarkers as contemplated herein encompass a broad range of data informative of patient health. Dried blood or dried plasma is an exemplary source of biomarker information, but a broad range of biomarkers and biomarker sources are compatible with the disclosure herein. In various embodiments, markers contemplated herein include at least one of patient age, gender, glucose level, blood pressure, sleep patterns, weight measurements, calorie intake, food intake constituents, vitamin or pharmaceutical intake, prescription drug use patterns, substance abuse history, exercise patterns or exercise output quantification (in terms, for example, of distance, an estimate of calories consumed, or other measure of energy consumed or exerted), and biomolecule measurement.

Additional markers employed in some embodiments include the time and place at which a sample is collected, such as at least one of time of day, time of week, date, and season in which a sample is collected. Similarly, geographic information related to the location at which the sample is collected, and/or geographical information relating to the individual from which the sample is collected, is also included in some embodiments.

A biomolecule serving as a biomarker can be measured from a sample in any number of patient tissues, for example fluids such as in at least one of a patient's blood, blood serum, urine, saliva, cerebrospinal fluid, breath exudate or any number of other tissues or fluids. In some cases, biomolecules are measured in, for example, patient urine, collected particles or fluid droplets in breath, or in saliva or blood. Preferred embodiments comprise measurement of a plurality of biomarkers from patient blood, such as protein biomarkers.

Biomarkers derived from a patient sample such as a patient fluid, for example as circulating biomarkers in patient blood, are quantified through a number of approaches consistent with the disclosure herein. When specific markers are targeted for measurement, mass spectrometric approaches or antibodies are used to detect and in some cases to quantify the level of at least one biomarker in a sample. Alternately or in combination, biomarkers such as circulating biomarkers in a blood sample or biomarkers obtained from breath aspirate are quantified, either relatively or absolutely, through mass spectrometric approaches.

Some aspects of the approaches described herein include the generation of large amounts of biomarker measurements. In various embodiments, measurements are made so that levels are determined for at least 5, 6, 7, 8, 9, 10, 11, 12, 13, 14, 15, 16, 17, 18, 19, 20, 25, 30, 35, 40, 45, 50, 60, 70, 80, 90, 100, 150, or 200 or more biomarkers in a sample.

In some examples, label-free, label, or any other mass-shifted techniques are used to identify or quantify molecular markers in the sample. For example, label-free techniques include but are not limited to the Stable Isotope Standard (SIS) peptide response. Label techniques include but are not limited to chemical or enzymatic tagging of peptides or proteins. In some examples molecular markers in the sample include all the proteins associated with a particular disease. In some examples, these proteins are selected based on several performance characteristics (i.e. peak abundance, CV's, precision, etc.).

As disclosed herein, biomarkers can be accurately and repeatably measured for analyses such as in comparison to reference levels. Reference levels include levels of biomarkers determined from average levels of a plurality of individuals or samples for which at least one health condition status is known. Alternately or in combination, reference levels of biomarkers are determined from samples taken from the same individual at different times, such that temporal changes in an individual's biomarker profile are observed over time and such that a change in at least one up to a large number of biomarkers associated with a health status or condition is indicative of a change or an upcoming change in that health status or condition.

In some cases, a single biomarker is indicative of a health status in some instances, such that a change in the biomarker level is informative as to a change in health status. Alternately or in combination, a number of biomarkers, even if individually not informative of health status or informative below a confidence level upon which information is actionable, may exhibit changes in concert such that a health condition or status for which they are commonly implicated is identified as being altered or likely to be altered in the future with a level of confidence warranting action.

Biomarker measurements can be generated from mass spectrometry data or other sources such as protein or peptide array or immunological assays. In some cases, the measurements are for biomarkers corresponding to at least one of 1) known proteins or fragments mapping to known proteins of known function and known role in at least one heath status or disorder, 2) known proteins or known fragments mapping to known proteins of known function but unknown role in a health status or disorder, 3) unknown or unidentified proteins or fragments, such as fragments that have not been mapped to or identified with a particular protein of known function, but that nonetheless are in some cases relevant as markers for a health status or condition, for example due to their identifiable difference in levels between samples that differ in a known or hypothesized health status or health condition.

Accordingly, in various embodiments herein, marker data is useful in identifying a protein or set of proteins that differ between samples, such as individuals of differing health status or within a single individual at different time points, such that the identity of the biomarkers indicate a health condition or health status difference between individuals or in the individual at one time point compared to another. A non-limiting list of health conditions for which biomarkers are informative includes cardiovascular diseases (heart disease), hyperproliferative diseases (for example, cancer), neural diseases (for example, Alzheimer's disease), autoimmune diseases (for example, lupus metabolic diseases (such as obesity), inflammatory diseases (for example arthritis), bone diseases (such as osteoporosis) gastrointestinal diseases (such as ulcers), blood diseases (such as sickle cell anemia), infections (for example, bacterial, viral, and fungal infections), and chronic fatigue syndrome. Examples of hyperproliferative diseases such as cancer include colorectal, skin, lung, throat, blood, brain, breast, and prostate cancer.

Certain approaches described herein are targeted to the identification of colorectal cancer, adenoma, or polyp health status. For example, advanced colorectal cancer can be detected using a variety of techniques, and often include identifiable health symptoms such as rectal bleeding or bloody stool, change in bowel habits, weakness/fatigue, cramping, and weight loss. However, early stage colorectal cancer can be more difficult to detect. In some cases, the individual has not developed colorectal cancer and instead has a pre-CRC adenoma or polyp. Therefore, some of the methods described herein assess early stage colorectal cancer or pre-CRC using a biomarker panel recited herein such as, for example, A2GL, ALS, PTPRJ, and age.

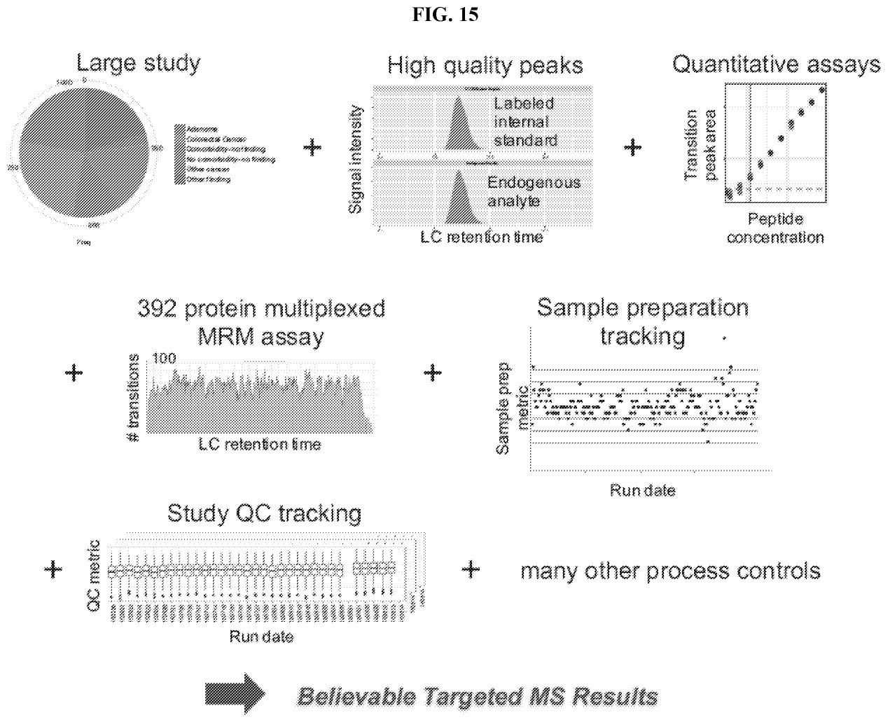

A diagram showing an approach for designing and characterizing a study to identify biomarkers suitable for use in assessing health status such as colorectal cancer status is shown in FIG. 15. The pie chart showing health conditions for various cases shows “other findings” starting from 0 to below 250, “other cancer” represented by a small slice below 250, “no comorbidity-no finding” starting just before 250 and extending to below 500, “comorbidity-no finding” represented by a slice that begins before 500 and extends past 500, “colorectal cancer” represented by a slice beginning past 500 and extending past 750, and “adenoma” beginning past 750 and extending until 1000.

### Quality Control Metrics

Described herein are quality control (QC) metrics informative of one or more factors having an influence on sample analysis. Such factors include sample collection, sample storage, sample elution, and other conditions or processes relevant to sample analysis. For example, certain conditions have an adverse impact on the quality, reliability, or variability of data that can be obtained from samples. Accordingly, QC metrics are indicative of at least one category of information such as sample integrity, sample elution efficiency, or filter storage condition. Sample integrity includes sample pH, sample stability, proteolytic activity, DNase activity, RNase activity, and other conditions informative of potential damage to the sample. Sample elution efficiency includes hydropathy-associated elution efficiency, overall sample elution efficiency, elution efficiency of sample constituents, and other indicators for assessing successful elution. Filter storage condition includes duration of sample storage, maximum temperature exposure, minimum temperature exposure, average temperature exposure, time-temperature exposure, light exposure, UV exposure, radiation exposure, humidity, and other conditions to which the sample has been exposed. QC metrics can be used to discard samples, discard or gate at least a portion of assay data obtained from the sample from further analysis or use in categorizing a result (e.g., CRC health status). For example, if a QC metric indicates that a threshold percentage of a marker of interest has failed to successfully elute from a collection device (e.g., greater than 10% of the marker or a corresponding internal standard or QC marker has failed to elute), then the marker may be discarded from use in categorizing a result. Alternatively, the quantification of the marker may be adjusted based on the QC metric (e.g., readjust calculated amount of marker to account for the predicted amount that was lost during elution).

QC metrics can be evaluated with the help of QC markers that provide information indicative of one or more category of information. In some embodiments, a QC marker is indicative of duration of sample storage, maximum temperature exposure, minimum temperature exposure, average temperature exposure, time-temperature exposure, sample pH, light exposure, UV exposure, radiation exposure, humidity, elution efficiency of sample constituents, hydropathy-associated elution efficiency, overall sample elution efficiency, sample stability, proteolytic activity, DNase activity, or RNase activity. Non-limiting examples of QC markers include elution markers, humidity markers, pH markers, temperature markers, time markers, proteolysis markers, nuclease markers, stability markers, radiation markers, UV markers, and light markers. Examples of QC markers can be found in international application PCT/US2018/049583, which is hereby incorporated by reference in its entirety. Specifically, at least the description of elution markers, humidity markers, pH markers, temperature markers, time markers, proteolysis markers, nuclease markers, stability markers, radiation markers, UV markers, and light markers from PCT/US2018/049583 are hereby incorporated by reference.

In some cases, the QC markers are collected and/or stored together with the sample. For example, a collection device such as a filter paper or dried blood spot filter comprising at least one QC marker is contemplated herein. Alternatively or in combination, QC markers are added to the sample after collection but before or during sample processing or analysis. Collection devices are suitable for collecting or receiving a variety of samples. Suitable samples include liquid samples such as blood, saliva, urine, tears, lymph, bile, sputum, or other biological fluids. A filter often comprises at least one layer such as a porous layer impermeable to particulates. When QC markers are used, at least one QC marker is disposed on a collection device such as a filter during device assembly, after device assembly, prior to sample deposition, during sample deposition, after sample deposition, before sample elution, during sample elution, after sample elution, before sample processing (e.g., for mass spectrometry or affinity assay analysis), during sample processing, or any combination thereof. At least one QC marker disposed on a collection device is positioned so as to co-migrate with a sample deposited on the device, co-elute from the filter with the sample, be stored on the device together with the sample, or any combination thereof. Alternatively, at least one QC marker disposed on a collection device is positioned to avoid co-elution with the sample. For example, some quality control markers provide direct information about the sample itself, which can include pH, proteolytic activity, or nuclease activity.

A filter consistent with the use of QC markers is a Noviplex Plasma Prep Card (Novilytic Labs), which comprises multiple layers that include an overlay (surface layer), a spreading layer, a separator (for filtering cells), a plasma collection reservoir, an isolation card, and a base card. In these types of filters, at least one QC marker can be disposed on at least one of the overlay, the spreading layer, the separator, the plasma collection reservoir, and the plasma collection reservoir. Variations on filter structure are contemplated, and markers and methods are compatible with a broad range of filter structures.

A QC marker can be positioned on a collection device based on the information the marker is intended to provide. For example, a marker for measuring the efficiency of sample migration from the overlay (surface) to the plasma collection reservoir is positioned on the overlay such that it co-migrates with the sample to the reservoir following sample deposition on the filter. Quantifying the marker in eluted sample relative to a marker in the collection reservoir, for example, can provide the elution efficiency of the device.

The corresponding marker, for example, having a known mass spectrometry migration offset (e.g., due to isotope labeling or a chemical modification) can be positioned in the reservoir at a known quantity. In certain cases, both markers have a known migration offset from a endogenous molecule from the sample to allow differentiation from the endogenous molecule. After sample elution, the two markers can be quantified using mass spectrometry to determine a ratio representative of the amount or proportion of the marker that is “lost” during sample migration. This, in turn, provides an estimate of the loss of the sample or biomarker in the sample collection process. Alternatively, when at least one QC marker indicates that only a subset of the data is impaired or compromised, the sample data is optionally gated to remove the compromised subset while retaining the remaining data for subsequent analysis. For example, a QC marker may indicate temperature exposure exceeding a threshold that is predicted or known to result in degradation for certain temperature-sensitive proteins. Accordingly, the temperature-sensitive proteins or data corresponding to these proteins can be screened out from further analysis without losing the entire sample or data set.

Internal standards can be used to evaluate a QC metric. An internal standard can be used to generate a calibration curve of multiple dilutions of a known amount of a marker. This calibration curve can be used to evaluate the sensitivity, dynamic range, and other indicators of the assay performance. For example, a calibration curve may indicate a loss of signal when the quantity of a marker is below a certain threshold. This information can be used to adjust the assay or sample processing as described above such as, for example, discarding the sample and/or gating or removing data for markers that fall below the threshold.

### Machine Learning

Some embodiments involve machine learning as a component of database analysis, and accordingly some computer systems are configured to comprise a module having a machine learning capacity. Machine learning modules often comprise at least one of the following listed modalities, so as to constitute a machine learning functionality.

Modalities that constitute machine learning variously demonstrate a data filtering capacity, so as to be able to perform automated mass spectrometric data spot detection and calling. This modality is in some cases facilitated by the presence of marker polypeptides, such as heavy isotope labeled polypeptides or other markers in a mass spectrometric analysis output, so that native peptides are readily identified and in some cases quantified. The markers are optionally added to samples prior to proteolytic digestion or subsequent to proteolytic digestion. Markers are in some embodiments present on a solid backing onto which a blood spot or other sample is deposited for storage or transfer prior to analysis via mass spectroscopy.

Modalities that constitute machine learning variously demonstrate a data treatment or data processing capacity, so as to render called data spots in a form conducive to downstream analysis. Examples of data treatment include but are not necessarily limited to log transformation, assigning of scaling ratios, or mapping data to crafted features so as to render the data in a form that is conducive to downstream analysis.

Machine learning data analysis components as disclosed herein regularly process a wide range of features in a mass spectrometric data set, such as 1 to 10,000 features, or 2 to 300,000 features, or a number of features within either of these ranges or higher than either of these ranges. In some cases, data analysis involves at least 1k, 2k, 3k, 4k, 5k, 6k, 7k, 8k, 9k, 10k, 20k, 30k, 40k, 50k, 60k, 70k, 80k, 90k, 100k, 120k, 140k, 160k, 180k, 200k, 220k, 2240k, 260k, 280k, 300k, or more than 300k features.

Features are selected using any number of approaches consistent with the disclosure herein. In some cases, feature selection comprises elastic net, information gain, random forest imputing or other feature selection approaches consistent with the disclosure herein and familiar to one of skill in the art.

Selected feature are assembled into classifiers, again using any number of approaches consistent with the disclosure herein. In some cases, classifier generation comprises logistic regression, SVM, random forest, KNN, or other classifier approaches consistent with the disclosure herein and familiar to one of skill in the art.

Machine learning approaches variously comprise implementation of at least one approach selected from the list consisting of ADTree, BFTree, ConjunctiveRule, DecisionStump, Filtered Classifier, J48, J48Graft, JRip, LADTree, NNge, OneR, OrdinalClassClassifier, PART, Ridor, SimpleCart, Random Forest and SVM.

Applying machine learning, or providing a machine learning module on a computer configured for the analyses disclosed herein, allows for the detection of relevant panels for asymptomatic disease detection or early detection as part of an ongoing monitoring procedure, so as to identify a disease or disorder either ahead of symptom development or while intervention is either more easily accomplished or more likely to bring about a successful outcome. Monitoring is often but not necessarily performed in combination with or in support of a genetic assessment indicating a genetic predisposition for a disorder for which a signature of onset or progression is monitored. Similarly, in some cases machine learning is used to facilitate monitoring of or assessment of treatment efficacy for a treatment regimen, such that the treatment regimen can be modified over time, continued or resolved as indicated by the ongoing proteomics mediated monitoring.

Machine learning approaches and computer systems having modules configured to execute machine learning algorithms facilitate identification of classifiers or panels in datasets of varying complexity. In some cases the classifiers or panels are identified from an untargeted database comprising a large amount of mass spectrometric data, such as data obtained from a single individual at multiple time points, samples taken from multiple individuals such as multiple individuals of a known status for a condition of interest or known eventual treatment outcome or response, or from multiple time points and multiple individuals.

Alternately, in some cases machine learning facilitates the refinement of a panel through the analysis of a database targeted to that panel, by for example collecting panel information for that panel from a single individual over multiple time points, when a health condition for the individual is known for the time points, or collecting panel information from multiple individuals of known status for a condition of interest, or collecting panel information from multiple individuals at multiple time points. As is readily apparent, in some cases collection of panel information is facilitated through the use of mass markers, such as heavy-labeled or ‘light-labeled’ mass markers that migrate so as to identify nearby unlabeled spots corresponding to the marked polypeptides. Thus, panel information is collected either alone or in combination with untargeted mass spectrometric data collection. Panel data is subjected to machine learning, for example on a computer system configured as disclosed herein, so as to identify a subset of panel markers that either alone or in combination with one or more non-panel markers analyzed through an untargeted approach, account for a health status signal. Thus, machine learning in some cases facilitates identification of a panel that is individually informative of a health status in an individual.

### Dried Blood Spot Analysis

Methods, databases and computers configured to receive mass spectrometric data as disclosed herein often involve processing mass spectrometric data sets that are spatially, temporally or spatially and temporally large. That is, datasets are generated that in some cases comprise large amounts of mass spectrometric data points per sample collected, are generated from large numbers of collected samples, and are in some cases generated from multiple samples derived from a single individual.

Data collection is in some cases facilitated by depositing samples such as dried blood samples (or other readily obtained samples such as urine, sweat, saliva or other fluid or tissue) onto a solid framework such as a solid backing or solid three-dimensional framework. The sample such as a blood sample is deposited on the solid backing or framework, where it is actively or passively dried, facilitating storage or transport from a collection point to a location where it may be processed.

As disclosed herein, a number of approaches are available for recovering proteomic or other biomarker information from a dried sample such as a dried blood spot sample. In some cases samples are solubilized, for example in TFE, and subjected to proteolysis to generate fragments to be visualized by mass spectrometric analysis. Proteolysis is accomplished by enzymatic or non-enzymatic treatment. Exemplary proteases include trypsin, but also enzymes such as proteinase K, enteropeptidase, furin, liprotamase, bromelain, serratipeptidase, thermolysin, collagenase, plasmin, or any number of serine proteases, cysteine proteases or other specific or nonspecific enzymatic peptidases, used singly or in combination. Nonenzymatic protease treatments, such as high temperature, pH treatment, cyanogen bromide and other treatments are also consistent with some embodiments.

When particular mass spectrometric fragments are of interest or use in analysis, such as a biomarker panel indicative of a health condition status, it is often beneficial to include heavy-labeled or other markers as standard markers as described herein. Markers, as discussed, migrate on a mass spectrometric output at a known position and at a known offset relative to the sample fragments of interest. Inclusion of these markers often leads to ‘offset doublets’ in mass spectrometric output. By detecting these doublets, one can readily, either personally or through an automated data analysis workflow, identify particular spots of interest to a health condition status among and in addition to the full range of mass spectrometric output data. When the markers have known mass and amount, and optionally when the amount loaded into a sample varies among markers, the markers are also useful as mass standards, facilitating quantification of both the marker-associated fragments and the remaining fragments in the mass spectrometric output.

Standard markers are introduced to a sample either at collection, during or subsequent to resolubilization, prior to digestion or subsequent to digestion. That is, in some cases a sample collection structure such as a solid backing or a three-dimensional volume is ‘pre-loaded’ so as to have a standard marker or standard markers present prior to sample collection. Alternately, the standard markers are added to the collection structure subsequent to sample collection, subsequent to sample drying on the structure, during or subsequent to sample collection, during or subsequent to sample resolubilization, or during or subsequent to sample proteolysis treatment. In preferred embodiments, exactly or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95, 100, 110, 120, 130, 140, 150, 160, 170, 180, 190, 200, 225, 250, 275, 300, or more than 300 standard markers are added to a collection structure prior to sample collection, such that standard processing of the sample results in a mass spectrometric output having the standard markers included in the output without any additional processing of the sample. Accordingly, some methods disclosed herein comprise providing a collection device having sample markers introduced onto the surface prior to sample collection, and some devices or computer systems are configured to receive mass spectrometric data having standard markers included therein, and optionally to identify the mass spectrometric markers and their corresponding native mass fragment.

### Certain Definitions

As used in the specification and claims, the singular forms “a”, “an” and “the” include plural references unless the context clearly dictates otherwise. For example, the term “a sample” includes a plurality of samples, including mixtures thereof.

The terms “determining”, “measuring”, “evaluating”, “assessing,” “assaying,” and “analyzing” are often used interchangeably herein to refer to forms of measurement, and include determining if an element is present or not (for example, detection). These terms can include quantitative, qualitative or quantitative and qualitative determinations. Assessing is alternatively relative or absolute. “Detecting the presence of” includes determining the amount of something present, as well as determining whether it is present or absent.

The terms “panel”, “biomarker panel”, “protein panel” are used interchangeably herein to refer to a set of biomarkers, wherein the set of biomarkers comprises at least two biomarkers. Exemplary biomarkers are proteins or polypeptide fragments of proteins that are uniquely or confidently mapped to particular proteins. However, additional biomarkers are also contemplated, for example age or gender of the individual providing a sample. The biomarker panel is often predictive and/or informative of a subject's health status, disease, or condition.

The “level” of a biomarker panel refers to the absolute and relative levels of the panel's constituent markers and the relative pattern of the panel's constituent biomarkers.

The terms “colorectal cancer” and “CRC” are used interchangeably herein. The term “colorectal cancer status”, “CRC status” can refer to the status of the disease in subject. Examples of types of CRC statuses include, but are not limited to, the subject's risk of cancer, including colorectal carcinoma, the presence or absence of disease (for example, adenocarcinoma), the stage of disease in a patient (for example, carcinoma), and the effectiveness of treatment of disease. In some cases, a health status is the presence or absence of an adenoma or polyp that is pre-CRC.

The term “mass spectrometer” can refer to a gas phase ion spectrometer that measures a parameter that can be translated into mass-to-charge (m/z) ratios of gas phase ions. Mass spectrometers generally include an ion source and a mass analyzer. Examples of mass spectrometers are time-of-flight, magnetic sector, quadrupole filter, ion trap, ion cyclotron resonance, electrostatic sector analyzer and hybrids of these. “Mass spectrometry” can refer to the use of a mass spectrometer to detect gas phase ions.

The term “biomarker” and “marker” are used interchangeably herein, and can refer to a polypeptide, gene, nucleic acid (for example, DNA and/or RNA) which is differentially present in a sample taken from a subject having a disease for which a diagnosis is desired (for example, CRC), or to other data obtained from the subject with or without sample acquisition, such as patient age information or patient gender information, as compared to a comparable sample or comparable data taken from control subject that does not have the disease (for example, a person with a negative diagnosis or undetectable CRC, normal or healthy subject, or, for example, from the same individual at a different time point). Common biomarkers herein include proteins, or protein fragments that are uniquely or confidently mapped to a particular protein (or, in cases such as SAA, above, a pair or group of closely related proteins), transition ion of an amino acid sequence, or one or more modifications of a protein such as phosphorylation, glycosylation or other post-translational or co-translational modification. In addition, a protein biomarker can be a binding partner of a protein, protein fragment, or transition ion of an amino acid sequence.

The terms “polypeptide,” “peptide” and “protein” are often used interchangeably herein in reference to a polymer of amino acid residues. A protein, generally, refers to a full-length polypeptide as translated from a coding open reading frame, or as processed to its mature form, while a polypeptide or peptide informally refers to a degradation fragment or a processing fragment of a protein that nonetheless uniquely or identifiably maps to a particular protein. A polypeptide can be a single linear polymer chain of amino acids bonded together by peptide bonds between the carboxyl and amino groups of adjacent amino acid residues. Polypeptides can be modified, for example, by the addition of carbohydrate, phosphorylation, etc. Proteins can comprise one or more polypeptides.

An “immunoassay” is an assay that uses an antibody to specifically bind an antigen (for example, a marker). The immunoassay can be characterized by the use of specific binding properties of a particular antibody to isolate, target, and/or quantify the antigen.

An “aptamer assay” is an assay that uses an oligonucleotide (e.g., DNA, RNA, or a nucleic acid analogue such as peptide nucleic acid, morpholino, glycol nucleic acid, or threose nucleic acid) or a peptide molecule to specifically bind a target (for example, a protein or peptide biomarker). The aptamer assay can be characterized by the use of specific binding properties of a particular aptamer molecule to isolate, target, and/or quantify the target.

The term “antibody” can refer to a polypeptide ligand substantially encoded by an immunoglobulin gene or immunoglobulin genes, or fragments thereof, which specifically binds and recognizes an epitope. Antibodies exist, for example, as intact immunoglobulins or as a number of well-characterized fragments produced by digestion with various peptidases.

The term “tumor” can refer to a solid or fluid-filled lesion or structure that may be formed by cancerous or non-cancerous cells, such as cells exhibiting aberrant cell growth or division. The terms “mass” and “nodule” are often used synonymously with “tumor”. Tumors include malignant tumors or benign tumors. An example of a malignant tumor can be a carcinoma which is known to comprise transformed cells.

The terms “subject,” “individual,” or “patient” are often used interchangeably herein. A “subject” can be a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. The subject may be diagnosed or suspected of being at high risk for a disease. The disease can be cancer. The cancer can be CRC (CRC). In some cases, the subject is not necessarily diagnosed or suspected of being at high risk for the disease.

The term specificity, or true negative rate, can refer to a test's ability to exclude a condition correctly. For example, in a diagnostic test, the specificity of a test is the proportion of patients known not to have the disease, who will test negative for it. In some cases, this is calculated by determining the proportion of true negatives (i.e. patients who test negative who do not have the disease) to the total number of healthy individuals in the population (i.e., the sum of patients who test negative and do not have the disease and patients who test positive and do not have the disease).

The term sensitivity, or true positive rate, can refer to a test's ability to identify a condition correctly. For example, in a diagnostic test, the sensitivity of a test is the proportion of patients known to have the disease, who will test positive for it. In some cases, this is calculated by determining the proportion of true positives (i.e. patients who test positive who have the disease) to the total number of individuals in the population with the condition (i.e., the sum of patients who test positive and have the condition and patients who test negative and have the condition).

The quantitative relationship between sensitivity and specificity can change as different diagnostic cut-offs are chosen. This variation can be represented using ROC curves. The x-axis of a ROC curve shows the false-positive rate of an assay, which can be calculated as (1−specificity). The y-axis of a ROC curve reports the sensitivity for an assay. This allows one to easily determine a sensitivity of an assay for a given specificity, and vice versa.

As used herein, the term ‘about’ a number refers to that number plus or minus 10% of that number. The term ‘about’ a range refers to that range minus 10% of its lowest value and plus 10% of its greatest value.

As used herein, the terms “treatment” or “treating” are used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

### Digital Processing Device

In some embodiments, the platforms, systems, media, and methods described herein include a digital processing device, or use of the same. In further embodiments, the digital processing device includes one or more hardware central processing units (CPUs) or general purpose graphics processing units (GPGPUs) that carry out the device's functions. In still further embodiments, the digital processing device further comprises an operating system configured to perform executable instructions. In some embodiments, the digital processing device is optionally connected a computer network. In further embodiments, the digital processing device is optionally connected to the Internet such that it accesses the World Wide Web. In still further embodiments, the digital processing device is optionally connected to a cloud computing infrastructure. In other embodiments, the digital processing device is optionally connected to an intranet. In other embodiments, the digital processing device is optionally connected to a data storage device.

In accordance with the description herein, suitable digital processing devices include, by way of non-limiting examples, server computers, desktop computers, laptop computers, notebook computers, sub-notebook computers, netbook computers, netpad computers, set-top computers, media streaming devices, handheld computers, Internet appliances, mobile smartphones, tablet computers, personal digital assistants, video game consoles, and vehicles. Those of skill in the art will recognize that many smartphones are suitable for use in the system described herein. Those of skill in the art will also recognize that select televisions, video players, and digital music players with optional computer network connectivity are suitable for use in the system described herein. Suitable tablet computers include those with booklet, slate, and convertible configurations, known to those of skill in the art.

In some embodiments, the digital processing device includes an operating system configured to perform executable instructions. The operating system is, for example, software, including programs and data, which manages the device's hardware and provides services for execution of applications. Those of skill in the art will recognize that suitable server operating systems include, by way of non-limiting examples, FreeBSD, OpenBSD, NetBSD®, Linux, Apple® Mac OS X Server®, Oracle® Solaris®, Windows Server®, and Novell® NetWare®. Those of skill in the art will recognize that suitable personal computer operating systems include, by way of non-limiting examples, Microsoft® Windows®, Apple® Mac OS X®, UNIX®, and UNIX-like operating systems such as GNU/Linux®. In some embodiments, the operating system is provided by cloud computing. Those of skill in the art will also recognize that suitable mobile smart phone operating systems include, by way of non-limiting examples, Nokia® Symbian® OS, Apple® iOS®, Research In Motion® BlackBerry OS®, Google® Android®, Microsoft® Windows Phone® OS, Microsoft® Windows Mobile® OS, Linux®, and Palm® WebOS®. Those of skill in the art will also recognize that suitable media streaming device operating systems include, by way of non-limiting examples, Apple TV®, Roku®, Boxee®, Google TV®, Google Chromecast®, Amazon Fire®, and Samsung® HomeSync®. Those of skill in the art will also recognize that suitable video game console operating systems include, by way of non-limiting examples, Sony® PS3®, Sony® PS4®, Microsoft® Xbox 360®, Microsoft Xbox One, Nintendo® Wii®, Nintendo® Wii U®, and Ouya®.

In some embodiments, the device includes a storage and/or memory device. The storage and/or memory device is one or more physical apparatuses used to store data or programs on a temporary or permanent basis. In some embodiments, the device is volatile memory and requires power to maintain stored information. In some embodiments, the device is non-volatile memory and retains stored information when the digital processing device is not powered. In further embodiments, the non-volatile memory comprises flash memory. In some embodiments, the non-volatile memory comprises dynamic random-access memory (DRAM). In some embodiments, the non-volatile memory comprises ferroelectric random access memory (FRAM). In some embodiments, the non-volatile memory comprises phase-change random access memory (PRAM). In other embodiments, the device is a storage device including, by way of non-limiting examples, CD-ROMs, DVDs, flash memory devices, magnetic disk drives, magnetic tapes drives, optical disk drives, and cloud computing based storage. In further embodiments, the storage and/or memory device is a combination of devices such as those disclosed herein.

In some embodiments, the digital processing device includes a display to send visual information to a user. In some embodiments, the display is a cathode ray tube (CRT). In some embodiments, the display is a liquid crystal display (LCD). In further embodiments, the display is a thin film transistor liquid crystal display (TFT-LCD). In some embodiments, the display is an organic light emitting diode (OLED) display. In various further embodiments, on OLED display is a passive-matrix OLED (PMOLED) or active-matrix OLED (AMOLED) display. In some embodiments, the display is a plasma display. In other embodiments, the display is a video projector. In still further embodiments, the display is a combination of devices such as those disclosed herein.

In some embodiments, the digital processing device includes an input device to receive information from a user. In some embodiments, the input device is a keyboard. In some embodiments, the input device is a pointing device including, by way of non-limiting examples, a mouse, trackball, track pad, joystick, game controller, or stylus. In some embodiments, the input device is a touch screen or a multi-touch screen. In other embodiments, the input device is a microphone to capture voice or other sound input. In other embodiments, the input device is a video camera or other sensor to capture motion or visual input. In further embodiments, the input device is a Kinect, Leap Motion, or the like. In still further embodiments, the input device is a combination of devices such as those disclosed herein.

### Non-Transitory Computer Readable Storage Medium

In some embodiments, the platforms, systems, media, and methods disclosed herein include one or more non-transitory computer readable storage media encoded with a program including instructions executable by the operating system of an optionally networked digital processing device. In further embodiments, a computer readable storage medium is a tangible component of a digital processing device. In still further embodiments, a computer readable storage medium is optionally removable from a digital processing device. In some embodiments, a computer readable storage medium includes, by way of non-limiting examples, CD-ROMs, DVDs, flash memory devices, solid state memory, magnetic disk drives, magnetic tape drives, optical disk drives, cloud computing systems and services, and the like. In some cases, the program and instructions are permanently, substantially permanently, semi-permanently, or non-transitorily encoded on the media.

### Computer Program

In some embodiments, the platforms, systems, media, and methods disclosed herein include at least one computer program, or use of the same. A computer program includes a sequence of instructions, executable in the digital processing device's CPU, written to perform a specified task. Computer readable instructions may be implemented as program modules, such as functions, objects, Application Programming Interfaces (APIs), data structures, and the like, that perform particular tasks or implement particular abstract data types. In light of the disclosure provided herein, those of skill in the art will recognize that a computer program may be written in various versions of various languages.

The functionality of the computer readable instructions may be combined or distributed as desired in various environments. In some embodiments, a computer program comprises one sequence of instructions. In some embodiments, a computer program comprises a plurality of sequences of instructions. In some embodiments, a computer program is provided from one location. In other embodiments, a computer program is provided from a plurality of locations. In various embodiments, a computer program includes one or more software modules. In various embodiments, a computer program includes, in part or in whole, one or more web applications, one or more mobile applications, one or more standalone applications, one or more web browser plug-ins, extensions, add-ins, or add-ons, or combinations thereof.

### WEB Application

In some embodiments, a computer program includes a web application. In light of the disclosure provided herein, those of skill in the art will recognize that a web application, in various embodiments, utilizes one or more software frameworks and one or more database systems. In some embodiments, a web application is created upon a software framework such as Microsoft®.NET or Ruby on Rails (RoR). In some embodiments, a web application utilizes one or more database systems including, by way of non-limiting examples, relational, non-relational, object oriented, associative, and XML database systems. In further embodiments, suitable relational database systems include, by way of non-limiting examples, Microsoft® SQL Server, mySQL™, and Oracle®. Those of skill in the art will also recognize that a web application, in various embodiments, is written in one or more versions of one or more languages. A web application may be written in one or more markup languages, presentation definition languages, client-side scripting languages, server-side coding languages, database query languages, or combinations thereof. In some embodiments, a web application is written to some extent in a markup language such as Hypertext Markup Language (HTML), Extensible Hypertext Markup Language (XHTML), or eXtensible Markup Language (XML). In some embodiments, a web application is written to some extent in a presentation definition language such as Cascading Style Sheets (CSS). In some embodiments, a web application is written to some extent in a client-side scripting language such as Asynchronous Javascript and XML (AJAX), Flash® Actionscript, Javascript, or Silverlight®. In some embodiments, a web application is written to some extent in a server-side coding language such as Active Server Pages (ASP), ColdFusion®, Perl, Java™, JavaServer Pages (JSP), Hypertext Preprocessor (PHP), Python™, Ruby, Tcl, Smalltalk, WebDNA®, or Groovy. In some embodiments, a web application is written to some extent in a database query language such as Structured Query Language (SQL). In some embodiments, a web application integrates enterprise server products such as IBM® Lotus Domino®. In some embodiments, a web application includes a media player element. In various further embodiments, a media player element utilizes one or more of many suitable multimedia technologies including, by way of non-limiting examples, Adobe® Flash®, HTML 5, Apple® QuickTime®, Microsoft® Silverlight®, Java™, and Unity®.

### Mobile Application

In some embodiments, a computer program includes a mobile application provided to a mobile digital processing device. In some embodiments, the mobile application is provided to a mobile digital processing device at the time it is manufactured. In other embodiments, the mobile application is provided to a mobile digital processing device via the computer network described herein.

In view of the disclosure provided herein, a mobile application is created by techniques known to those of skill in the art using hardware, languages, and development environments known to the art. Those of skill in the art will recognize that mobile applications are written in several languages. Suitable programming languages include, by way of non-limiting examples, C, C++, C #, Objective-C, Java™, Javascript, Pascal, Object Pascal, Python™, Ruby, VB.NET, WML, and XHTML/HTML with or without CSS, or combinations thereof.

Suitable mobile application development environments are available from several sources. Commercially available development environments include, by way of non-limiting examples, AirplaySDK, alcheMo, Appcelerator®, Celsius, Bedrock, Flash Lite, .NET Compact Framework, Rhomobile, and WorkLight Mobile Platform. Other development environments are available without cost including, by way of non-limiting examples, Lazarus, MobiFlex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Those of skill in the art will recognize that several commercial forums are available for distribution of mobile applications including, by way of non-limiting examples, Apple® App Store, Google® Play, Chrome Web Store, BlackBerry® App World, App Store for Palm devices, App Catalog for webOS, Windows® Marketplace for Mobile, Ovi Store for Nokia® devices, Samsung® Apps, and Nintendo® DSi Shop.

### Standalone Application

In some embodiments, a computer program includes a standalone application, which is a program that is run as an independent computer process, not an add-on to an existing process, e.g., not a plug-in. Those of skill in the art will recognize that standalone applications are often compiled. A compiler is a computer program(s) that transforms source code written in a programming language into binary object code such as assembly language or machine code. Suitable compiled programming languages include, by way of non-limiting examples, C, C++, Objective-C, COBOL, Delphi, Eiffel, Java™, Lisp, Python™, Visual Basic, and VB .NET, or combinations thereof. Compilation is often performed, at least in part, to create an executable program. In some embodiments, a computer program includes one or more executable complied applications.

### Web Browser Plug-in

In some embodiments, the computer program includes a web browser plug-in (e.g., extension, etc.). In computing, a plug-in is one or more software components that add specific functionality to a larger software application. Makers of software applications support plug-ins to enable third-party developers to create abilities which extend an application, to support easily adding new features, and to reduce the size of an application. When supported, plug-ins enable customizing the functionality of a software application. For example, plug-ins are commonly used in web browsers to play video, generate interactivity, scan for viruses, and display particular file types. Those of skill in the art will be familiar with several web browser plug-ins including, Adobe® Flash® Player, Microsoft® Silverlight®, and Apple® QuickTime®. In some embodiments, the toolbar comprises one or more web browser extensions, add-ins, or add-ons. In some embodiments, the toolbar comprises one or more explorer bars, tool bands, or desk bands.

In view of the disclosure provided herein, those of skill in the art will recognize that several plug-in frameworks are available that enable development of plug-ins in various programming languages, including, by way of non-limiting examples, C++, Delphi, Java™ PHP, Python™, and VB .NET, or combinations thereof.

Web browsers (also called Internet browsers) are software applications, designed for use with network-connected digital processing devices, for retrieving, presenting, and traversing information resources on the World Wide Web. Suitable web browsers include, by way of non-limiting examples, Microsoft® Internet Explorer®, Mozilla® Firefox®, Google® Chrome, Apple® Safari®, Opera Software® Opera®, and KDE Konqueror. In some embodiments, the web browser is a mobile web browser. Mobile web browsers (also called mircrobrowsers, mini-browsers, and wireless browsers) are designed for use on mobile digital processing devices including, by way of non-limiting examples, handheld computers, tablet computers, netbook computers, subnotebook computers, smartphones, music players, personal digital assistants (PDAs), and handheld video game systems. Suitable mobile web browsers include, by way of non-limiting examples, Google® Android® browser, RIM BlackBerry® Browser, Apple® Safari®, Palm® Blazer, Palm® WebOS Browser, Mozilla® Firefox® for mobile, Microsoft® Internet Explorer® Mobile, Amazon Kindle Basic Web, Nokia® Browser, Opera Software® Opera® Mobile, and Sony® PSP™ browser.

### Software Modules

In some embodiments, the platforms, systems, media, and methods disclosed herein include software, server, and/or database modules, or use of the same. In view of the disclosure provided herein, software modules are created by techniques known to those of skill in the art using machines, software, and languages known to the art. The software modules disclosed herein are implemented in a multitude of ways. In various embodiments, a software module comprises a file, a section of code, a programming object, a programming structure, or combinations thereof. In further various embodiments, a software module comprises a plurality of files, a plurality of sections of code, a plurality of programming objects, a plurality of programming structures, or combinations thereof. In various embodiments, the one or more software modules comprise, by way of non-limiting examples, a web application, a mobile application, and a standalone application. In some embodiments, software modules are in one computer program or application. In other embodiments, software modules are in more than one computer program or application. In some embodiments, software modules are hosted on one machine. In other embodiments, software modules are hosted on more than one machine. In further embodiments, software modules are hosted on cloud computing platforms. In some embodiments, software modules are hosted on one or more machines in one location. In other embodiments, software modules are hosted on one or more machines in more than one location.

### Databases

In some embodiments, the platforms, systems, media, and methods disclosed herein include one or more databases, or use of the same. In view of the disclosure provided herein, those of skill in the art will recognize that many databases are suitable for storage and retrieval of biomarker information. In various embodiments, suitable databases include, by way of non-limiting examples, relational databases, non-relational databases, object oriented databases, object databases, entity-relationship model databases, associative databases, and XML databases. Further non-limiting examples include SQL, PostgreSQL, MySQL, Oracle, DB2, and Sybase. In some embodiments, a database is internet-based. In further embodiments, a database is web-based. In still further embodiments, a database is cloud computing-based. In other embodiments, a database is based on one or more local computer storage devices.

### Numbered Embodiments

The following embodiments recite nonlimiting permutations of combinations of features disclosed herein. Other permutations of combinations of features are also contemplated. 1. A method of assessing a colorectal health risk status in an individual, comprising steps of obtaining a circulating blood sample from said individual; and obtaining a biomarker panel level for at least one of A2GL, ALS, PTPRJ, and age of said individual, and assessing colorectal health risk status. 2. A method of analyzing a biological sample, comprising: obtaining protein levels in said biological sample for each protein of a biomarker panel comprising A2GL, ALS, and PTPRJ to determine a panel information for said biomarker panel; comparing said panel information to a reference panel information, wherein said reference panel information corresponds to a known colorectal cancer status; and categorizing said biological sample as having a positive colorectal cancer risk status if said panel information does not differ significantly from said reference panel information, wherein said biological sample is derived from a circulating blood sample. 3. The method of embodiment 2, wherein said biomarker panel further comprises at least one of an individual age and an individual gender. 4. The method of embodiment 2, wherein said known colorectal cancer status comprises at least one of early CRC and advanced CRC. 5. The method of embodiment 2, wherein said known colorectal cancer status comprises at least one of advanced adenoma, Stage 0 CRC, stage I CRC, Stage II CRC, stage III CRC, and stage IV CRC. 6. The method of embodiment 2, wherein said biomarker panel comprises no more than 20 proteins. 7. The method of embodiment 2, wherein said biomarker panel comprises no more than 10 proteins. 8. The method of embodiment 2, wherein said categorizing has a sensitivity of at least 70% and a specificity of at least 70%, or a sensitivity of at least 81% and a specificity of at least 78%. 9. The method of embodiment 2, further comprising performing a treatment regimen in response to said categorizing. 10. The method of embodiment 9, wherein said treatment regimen comprises at least one of chemotherapy, radiation, immunotherapy, administration of a biologic therapeutic agent, polypectomy, partial colectomy, low anterior resection or abdominoperineal resection and colostomy. 11. The method of embodiment 2, further comprising transmitting a report of results of said categorizing to a health practitioner. 12. The method of embodiment 11, wherein said report indicates a sensitivity of at least 70% or at least 81%. 13. The method of embodiment 11, wherein said report indicates a specificity of at least 70% or at least 78%. 14. The method of embodiment 11, wherein said report indicates a recommendation for a treatment regimen comprising at least one of chemotherapy, radiation, immunotherapy, administration of a biologic therapeutic agent, polypectomy, partial colectomy, low anterior resection or abdominoperineal resection and colostomy. 15. The method of embodiment 11, wherein said report indicates a recommendation for a colonoscopy. 16. The method of embodiment 11, wherein said report indicates a recommendation for undergoing an independent cancer assay. 17. The method of embodiment 11, wherein said report indicates a recommendation for undergoing a stool cancer assay. 18. The method of embodiment 2, further comprising performing a stool cancer assay in response to said categorizing. 19. The method of embodiment 2, further comprising continued monitoring for a period of 3 months or greater. 20. The method of embodiment 2, further comprising continued monitoring for a period of between 3 months and 24 months. 21. The method of embodiment 2, wherein said obtaining said protein levels comprises subjecting said biological sample to a mass spectrometric analysis. 22. The method of embodiment 2, wherein said obtaining said protein levels comprises subjecting said biological sample to an immunoassay analysis. 23. A method of analyzing a biological sample, comprising: obtaining protein levels in said biological sample for each protein of a biomarker panel comprising A2GL, ALS, and PTPRJ to determine a panel information for said biomarker panel; comparing said panel information to a reference panel information, wherein said reference panel information corresponds to a known advanced adenoma status; and categorizing said blood sample as having a positive advanced adenoma risk status if said panel information does not differ significantly from said reference panel information, wherein said biological sample is derived from a circulating blood sample. 24. The method of embodiment 23, wherein said biomarker panel further comprises at least one of an individual age and an individual gender. 25. The method of embodiment 23, wherein said biomarker panel comprises no more than 20 proteins. 26. The method of embodiment 23, wherein said biomarker panel comprises no more than 10 proteins. 27. The method of embodiment 23, wherein said categorizing has a sensitivity of at least 44% and a specificity of at least 80%. 28. The method of embodiment 23, further comprising performing a treatment regimen in response to said categorizing. 29. The method of embodiment 28, wherein said treatment regimen comprises at least one of chemotherapy, radiation, immunotherapy, administration of a biologic therapeutic agent, polypectomy, partial colectomy, low anterior resection or abdominoperineal resection and colostomy. 30. The method of embodiment 23, comprising transmitting a report of results of said categorizing to a health practitioner. 31. The method of embodiment 30, wherein said report indicates a sensitivity of at least 70% or at least 81%. 32. The method of embodiment 30, wherein said report indicates a specificity of at least 70% or at least 87%. 33. The method of embodiment 30, wherein said report indicates a recommendation for a treatment regimen comprising at least one of chemotherapy, radiation, immunotherapy, administration of a biologic therapeutic agent, polypectomy, partial colectomy, low anterior resection or abdominoperineal resection and colostomy. 34. The method of embodiment 30, wherein said report indicates a recommendation for a colonoscopy. 35. The method of embodiment 30, wherein said report indicates a recommendation for undergoing an independent cancer assay. 36. The method of embodiment 30, wherein said report indicates a recommendation for undergoing a stool cancer assay. 37. The method of embodiment 23, further comprising performing a stool cancer assay. 38. The method of embodiment 23, further comprising continued monitoring for a period of 3 months or greater. 39. The method of embodiment 23, further comprising continued monitoring for a period of between 3 months and 24 months. 40. The method of embodiment 23, wherein obtaining said protein levels comprises subjecting said biological sample to a mass spectrometric analysis. 41. The method of embodiment 23, wherein said obtaining said protein levels comprises subjecting said biological sample to an immunoassay analysis. 42. A method of analyzing data generated in vitro, comprising: storing, by a processor, a panel information corresponding to a biological sample, wherein said panel information comprises protein levels for each protein of a biomarker panel comprising A2GL, ALS, and PTPRJ; comparing, by said processor, said panel information to a reference panel information, wherein said reference panel information corresponds to a known colorectal cancer status; and categorizing, by said processor, said panel information as having a positive colorectal cancer risk status if said panel information does not differ significantly from said reference panel information. 43. The method of embodiment 42, wherein said biomarker panel further comprises at least one of an individual age and an individual gender. 44. The method of embodiment 42, wherein said known colorectal cancer status comprises at least one of early CRC and advanced CRC. 45. The method of embodiment 42, wherein said known colorectal cancer status comprises at least one of advanced adenoma, Stage 0 CRC, stage I CRC, Stage II CRC, stage III CRC, and stage IV CRC. 46. The method of embodiment 42, wherein said biomarker panel comprises no more than 20 proteins. 47. The method of embodiment 42, wherein said biomarker panel comprises no more than 10 proteins. 48. The method of embodiment 42, wherein said categorizing has a sensitivity of at least 70% and a specificity of at least 70%, or a sensitivity of at least 81% and a specificity of at least 78%. 49. The method of embodiment 42, wherein said processor is further configured to generate a report indicating said positive colorectal cancer risk status. 50. The method of embodiment 49, wherein said report further indicates recommendation for a treatment regimen in response to said categorizing. 51. The method of embodiment 49, wherein said treatment regimen comprises at least one of chemotherapy, radiation, immunotherapy, administration of a biologic therapeutic agent, polypectomy, partial colectomy, low anterior resection or abdominoperineal resection and colostomy. 52. The method of embodiment 49, wherein said report indicates a sensitivity of at least 70% or at least 81%. 53. The method of embodiment 49, wherein said report indicates a specificity of at least 70% or at least 78%. 54. The method of embodiment 49, wherein said report indicates recommendation for a colonoscopy. 55. The method of embodiment 49, wherein said report indicates recommendation for undergoing an independent cancer assay. 56. The method of embodiment 49, wherein said report indicates recommendation for undergoing a stool cancer assay. 57. A method of analyzing data generated in vitro, comprising: storing a panel information comprising protein levels for each protein of a biomarker panel comprising A2GL, ALS, and PTPRJ; comparing said panel information to a reference panel information, wherein said reference panel information corresponds to a known advanced adenoma status; and categorizing said panel information as having a positive advance adenoma risk status if said panel information does not differ significantly from said reference panel information. 58. The method of embodiment 57, wherein said biomarker panel further comprises at least one of an individual age and an individual gender. 59. The method of embodiment 57, wherein said biomarker panel comprises no more than 20 proteins. 60. The method of embodiment 57, wherein said biomarker panel comprises no more than 10 proteins. 61. The method of embodiment 57, wherein said categorizing has a sensitivity of at least 70% and a specificity of at least 70%. 62. The method of embodiment 57, further comprising generating a report indicating said positive advanced adenoma status. 63. The method of embodiment 62, wherein said report further indicates recommendation for a treatment regimen in response to said categorizing. 64. The method of embodiment 63, wherein said treatment regimen comprises at least one of chemotherapy, radiation, immunotherapy, administration of a biologic therapeutic agent, polypectomy, partial colectomy, low anterior resection or abdominoperineal resection and colostomy. 65. The method of embodiment 62, wherein said report indicates a sensitivity of at least 70%. 66. The method of embodiment 62, wherein said report indicates a specificity of at least 70%. 67. The method of embodiment 62, wherein said report indicates recommendation for a colonoscopy. 68. The method of embodiment 62, wherein said report indicates recommendation for undergoing an independent cancer assay. 69. The method of embodiment 62, wherein said report indicates recommendation for undergoing a stool cancer assay. 70. A computer system for analyzing data generated in vitro, comprising: (a) a memory unit for receiving a panel information comprising measurement of protein levels of each protein in a biomarker panel from a biological sample, wherein the biomarker panel comprises A2GL, ALS, and PTPRJ; (b) computer-executable instructions for comparing said panel information to a reference panel information, wherein said reference panel information corresponds to a known colorectal cancer status; and (c) computer-executable instructions for categorizing said panel information as having a positive colorectal cancer status if said panel information does not differ significantly from said reference panel information. 71. The computer system of embodiment 70, further comprising computer-executable instructions to generate a report of said positive colorectal cancer status. 72. The computer system of embodiment 70, wherein said biomarker panel further comprises at least one of an individual age and an individual gender. 73. The computer system of embodiment 70, wherein said known colorectal cancer status comprises at least one of early CRC and advanced CRC. 74. The computer system of embodiment 70, wherein said known colorectal cancer status comprises at least one of advanced adenoma, Stage 0 CRC, stage I CRC, Stage II CRC, stage III CRC, and stage IV CRC. 75. The computer system of embodiment 70, wherein said biomarker panel comprises no more than 20 proteins. 76. The computer system of embodiment 70, wherein said biomarker panel comprises no more than 10 proteins. 77. The computer system of embodiment 70, wherein said categorizing has a sensitivity of at least 70% and a specificity of at least 70%. 78. The computer system of embodiment 70, further comprising generating a report indicating said positive colorectal cancer risk status. 79. The computer system of embodiment 78, wherein said report further indicates recommendation for a treatment regimen in response to said categorizing. 80. The computer system of embodiment 79, wherein said treatment regimen comprises at least one of chemotherapy, radiation, immunotherapy, administration of a biologic therapeutic agent, polypectomy, partial colectomy, low anterior resection or abdominoperineal resection and colostomy. 81. The computer system of embodiment 78, wherein said report indicates a sensitivity of at least 70%. 82. The computer system of embodiment 78, wherein said report indicates a specificity of at least 70%. 83. The computer system of embodiment 78, wherein said report indicates recommendation for a colonoscopy. 84. The computer system of embodiment 78, wherein said report indicates recommendation for undergoing an independent cancer assay. 85. The computer system of embodiment 79, wherein said report indicates recommendation for undergoing a stool cancer assay. 86. The computer system of embodiment 70, further comprising a user interface configured to communicate or display said report to a user. 87. A computer system for analyzing data generated in vitro: (a) a memory unit for receiving a panel information comprising measurement of protein levels of each protein in a biomarker panel from a biological sample, wherein said biomarker panel comprises A2GL, ALS, and PTPRJ; (b) computer-executable instructions for comparing said panel information to a reference panel information, wherein said reference panel information corresponds to a known advanced adenoma status; and (c) computer-executable instructions for categorizing said panel information as having a positive advanced adenoma status if said panel information does not differ significantly from said reference panel information. 88. The computer system of embodiment 87, wherein said biomarker panel further comprises at least one of an individual age and an individual gender. 89. The computer system of embodiment 87, wherein said biomarker panel comprises no more than 20 proteins. 90. The computer system of embodiment 87, wherein biomarker panel comprises no more than 10 proteins. 91. The computer system of embodiment 87, wherein said categorizing has a sensitivity of at least 70% and a specificity of at least 70%. 92. The computer system of embodiment 87, further comprising computer-executable instructions to generate a report of said positive advanced adenoma status. 93. The computer system of embodiment 92, wherein said report further indicates recommendation for a treatment regimen in response to said categorizing. 94. The computer system of embodiment 93, wherein said treatment regimen comprises at least one of chemotherapy, radiation, immunotherapy, administration of a biologic therapeutic agent, polypectomy, partial colectomy, low anterior resection or abdominoperineal resection and colostomy. 95. The computer system of embodiment 92, wherein said report indicates a sensitivity of at least 70%. 96. The computer system of embodiment 92, wherein said report indicates a specificity of at least 70%. 97. The computer system of embodiment 92, wherein said report indicates recommendation for a colonoscopy. 98. The computer system of embodiment 92, wherein said report indicates recommendation for undergoing an independent cancer assay. 99. The computer system of embodiment 92, wherein said report indicates recommendation for undergoing a stool cancer assay. 100. A method of assessing colorectal health of an individual, comprising: obtaining a circulating blood sample from said individual; and detecting protein levels for each member of a list of proteins in said sample, said list of proteins comprising A2GL, ALS, and PTPRJ. 101. The method of embodiment 100, further comprising diagnosing said individual as having a colorectal cancer status when said protein levels from said individual do not differ significantly from a reference panel information set corresponding to a known colorectal cancer risk status. 102. The method of embodiment 101, further comprising performing colonoscopy on said individual. 103. The method of embodiment 101, wherein said known colorectal cancer status comprises at least one of early CRC and advanced CRC. 104. The method of embodiment 101, wherein said known colorectal cancer status comprises at least one of advanced adenoma, Stage 0 CRC, stage I CRC, Stage II CRC, stage III CRC, and stage IV CRC. 105. The method of embodiment 101, further performing a treatment regimen upon said individual. 106. The method of embodiment 105, wherein said treatment regimen comprises a polypectomy. 107. The method of embodiment 105, wherein said treatment regimen comprises radiation. 108. The method of embodiment 105, wherein said treatment regimen comprises chemotherapy. 109. The method of embodiment 100, wherein said list of proteins further comprises at least one additional protein selected from Table 1. 110. The method of embodiment 100, wherein said list of proteins further comprises at least two additional proteins selected from Table 1. 111. The method of embodiment 100, wherein said list of proteins further comprises at least three additional proteins selected from Table 1. 112. The method of embodiment 100, further comprising obtaining at least one of an age and a gender of said individual. 113. The method of embodiment 100, further comprising transmitting a report to a health practitioner of results of said detecting. 114. The method of embodiment 113, wherein said report indicates recommendation for a colonoscopy for said individual. 115. The method of embodiment 113, wherein said report indicates recommendation for a polypectomy for said individual. 116. The method of embodiment 113, wherein said report indicates recommendation for radiation for said individual. 117. The method of embodiment 113, wherein said report indicates recommendation for chemotherapy for said individual. 118. The method of embodiment 113, wherein said report indicates recommendation for undergoing an independent cancer assay. 119. The method of embodiment 113, wherein said report indicates recommendation for undergoing a stool cancer assay. 120. The method of embodiment 100, wherein said list of proteins comprises no more than 20 proteins. 121. The method of embodiment 100, wherein said list of proteins comprises no more than 10 proteins. 122. A method of assessing colorectal health of an individual, comprising: obtaining a circulating blood sample from said individual; and detecting protein levels for each member of a list of proteins in said sample, said list of proteins comprising A2GL and ALS; and obtaining an age of said individual. 123. The method of embodiment 122, further comprising diagnosing said individual as having a colorectal cancer status when said protein levels from said individual do not differ significantly from a reference panel information set corresponding to a known colorectal cancer risk status. 124. The method of embodiment 123, further comprising performing colonoscopy on said individual. 125. The method of embodiment 123, wherein said known colorectal cancer status comprises at least one of early CRC and advanced CRC. 126. The method of embodiment 123, wherein said known colorectal cancer status comprises at least one of advanced adenoma, Stage 0 CRC, stage I CRC, Stage II CRC, stage III CRC, and stage IV CRC. 127. The method of embodiment 123, further performing a treatment regimen upon said individual. 128. The method of embodiment 127, wherein said treatment regimen comprises polypectomy. 129. The method of embodiment 127, wherein said treatment regimen comprises radiation. 130. The method of embodiment 127, wherein said treatment regimen comprises chemotherapy. 131. The method of embodiment 122, wherein said list of proteins further comprises PTPRJ. 132. The method of embodiment 122, wherein said list of proteins further comprises at least one additional protein selected from Table 1. 133. The method of embodiment 122, wherein said list of proteins further comprises at least two additional protein selected from Table 1. 134. The method of embodiment 122, wherein said list of proteins further comprises each additional protein selected from Table 1. 135. The method of embodiment 122, further comprising obtaining a gender of said individual. 136. The method of embodiment 122, further comprising transmitting a report to a health practitioner of results of said detecting. 137. The method of embodiment 136, wherein said report indicates recommendation for a colonoscopy for said individual. 138. The method of embodiment 136, wherein said report indicates recommendation for a polypectomy for said individual. 139. The method of embodiment 136, wherein said report indicates recommendation for radiation for said individual. 140. The method of embodiment 136, wherein said report indicates recommendation for chemotherapy for said individual. 141. The method of embodiment 136, wherein said report indicates recommendation for undergoing an independent cancer assay. 142. The method of embodiment 136, wherein said report indicates recommendation for undergoing a stool cancer assay. 143. The method of embodiment 122, wherein said list of proteins comprises no more than 15 proteins. 144. The method of embodiment 122, wherein said list of proteins comprises no more than 8 proteins. 145. A method of assessing colorectal health of an individual, comprising: obtaining a circulating blood sample from said individual; and detecting protein levels for each member of a list of proteins in the sample, said list of proteins comprising A2GL and ALS. 146. The method of embodiment 145, further comprising diagnosing said individual as having an advanced adenoma status when said protein levels from said individual do not differ significantly from a reference panel information set corresponding to a known advanced adenoma risk status. 147. The method of embodiment 146, further comprising performing colonoscopy on said individual. 148. The method of embodiment 146, further performing a treatment regimen upon said individual. 149. The method of embodiment 148, wherein said treatment regimen comprises polypectomy. 150. The method of embodiment 148, wherein said treatment regimen comprises radiation. 151. The method of embodiment 148, wherein said treatment regimen comprises chemotherapy. 152. The method of embodiment 145, wherein said list of proteins further comprises PTPRJ. 153. The method of embodiment 145, wherein said list of proteins further comprises at least one additional protein selected from Table 1. 154. The method of embodiment 145, wherein said list of proteins further comprises at least two additional proteins selected from Table 1. 155. The method of embodiment 145, wherein said list of proteins further comprises each additional protein selected from Table 1. 156. The method of embodiment 145, further comprising obtaining a gender of said individual. 157. The method of embodiment 145, further comprising transmitting a report to a health practitioner of results of said detecting. 158. The method of embodiment 157, wherein said report indicates recommendation for a colonoscopy for said individual. 159. The method of embodiment 157, wherein said report indicates recommendation for a polypectomy for said individual. 160. The method of embodiment 157, wherein said report indicates recommendation for radiation for said individual. 161. The method of embodiment 157, wherein said report indicates recommendation for chemotherapy for said individual. 162. The method of embodiment 157, wherein said report indicates recommendation for undergoing an independent cancer assay. 163. The method of embodiment 157, wherein said report indicates recommendation for undergoing a stool cancer assay. 164. The method of embodiment 145, wherein said list of proteins comprises no more than 15 proteins. 165. The method of embodiment 145, wherein said list of proteins comprises no more than 8 proteins. 166. A method of assessing colorectal health of an individual, comprising: obtaining a circulating blood sample from said individual; detecting protein levels for each member of a list of proteins in sample, said list of proteins comprising A2GL and ALS; and obtaining an age of said individual. 167. The method of embodiment 166, further comprising diagnosing said individual as having an advanced adenoma status when said protein levels from said individual do not differ significantly from a reference panel information set corresponding to a known advanced adenoma risk status. 168. The method of embodiment 167, further comprising performing colonoscopy on said individual. 169. The method of embodiment 167, further performing a treatment regimen upon said individual. 170. The method of embodiment 169, wherein said treatment regimen comprises polypectomy. 171. The method of embodiment 169, wherein said treatment regimen comprises radiation. 172. The method of embodiment 169, wherein said treatment regimen comprises chemotherapy. 173. The method of embodiment 166, wherein said list of proteins further comprises PTPRJ. 174. The method of embodiment 173, wherein said list of proteins further comprises at least one additional protein selected from Table 1. 175. The method of embodiment 166, further comprising obtaining a gender of said individual. 176. The method of embodiment 166, further comprising transmitting a report to a health practitioner of results of said detecting. 177. The method of embodiment 176, wherein said report indicates recommendation for a colonoscopy for said individual. 178. The method of embodiment 176, wherein said report indicates recommendation for a polypectomy for said individual. 179. The method of embodiment 176, wherein said report indicates recommendation for radiation for said individual. 180. The method of embodiment 176, wherein said report indicates recommendation for chemotherapy for said individual. 181. The method of embodiment 176, wherein said report indicates recommendation for undergoing an independent cancer assay. 182. The method of embodiment 176, wherein said report indicates recommendation for undergoing a stool cancer assay. 183. The method of embodiment 166, wherein said list of proteins comprises no more than 20 proteins. 184. The method of embodiment 166, wherein said list of proteins comprises no more than 10 proteins. 185. A method of assessing colorectal health of an individual, comprising: obtaining a circulating blood sample from said individual; detecting protein levels for each member of a list of proteins in sample, said list of proteins comprising A2GL and ALS. 186. The method of embodiment 185, further comprising diagnosing said individual as having a colorectal cancer status when said protein levels from said individual do not differ significantly from a reference panel information set corresponding to a known colorectal cancer risk status. 187. The method of embodiment 185 or 186, further comprising performing colonoscopy on said individual. 188. The method of any one of embodiments 185 to 187, further performing a treatment regimen upon said individual. 189. The method of embodiment 188, wherein said treatment regimen comprises polypectomy. 190. The method of embodiment 188, wherein said treatment regimen comprises radiation. 191. The method of embodiment 188, wherein said treatment regimen comprises chemotherapy. 192. The method of embodiment 185, wherein said list of proteins further comprises PTPRJ. 193. The method of embodiment 185, wherein said list of proteins further comprises at least one additional protein selected from Table 1. 194. The method of embodiment 185, comprising obtaining age information for said individual. 195. The method of embodiment 185, comprising obtaining gender information for said individual. 196. The method of embodiment 185, comprising obtaining age information and gender information for said individual. 197. The method of any one of embodiments 185 to 196, further comprising transmitting a report to a health practitioner of results of said detecting. 198. The method of any one of embodiments 195 to 197, further comprising diagnosing said individual as having a colorectal cancer status when said protein levels, age and gender from said individual as a whole do not differ significantly from a reference panel information set corresponding to a known colorectal cancer risk status. 199. The method of embodiment 185, wherein said report indicates recommendation for a colonoscopy for said individual. 200. The method of embodiment 197, wherein said report indicates recommendation for a polypectomy for said individual. 201. The method of embodiment 197, wherein said report indicates recommendation for radiation for said individual. 202. The method of embodiment 197, wherein said report indicates recommendation for chemotherapy for said individual. 203. The method of embodiment 197, wherein said report indicates recommendation for undergoing an independent cancer assay. 204. The method of embodiment 197, wherein said report indicates recommendation for undergoing a stool cancer assay. 205. The method of any one of embodiments 185 to 204, wherein said list of proteins comprises no more than 20 proteins. 206. The method of embodiment 185, wherein said list of proteins comprises no more than 10 proteins. 207. 208. A method of assessing colorectal health of an individual, comprising: obtaining a circulating blood sample from said individual; detecting protein levels for each member of a list of proteins in sample, said list of proteins comprising A2GL and ALS. 209. The method of embodiment 208, further comprising diagnosing said individual as having an advanced adenoma status when said protein levels from said individual do not differ significantly from a reference panel information set corresponding to a known advanced adenoma risk status. 210. The method of embodiment 208 or 209, further comprising performing colonoscopy on said individual. 211. The method of any one of embodiments 208 to 210, further performing a treatment regimen upon said individual. 212. The method of embodiment 211, wherein said treatment regimen comprises polypectomy. 213. The method of embodiment 211, wherein said treatment regimen comprises radiation. 214. The method of embodiment 211, wherein said treatment regimen comprises chemotherapy. 215. The method of embodiment 208, wherein said list of proteins further comprises PTPRJ. 216. The method of embodiment 208, wherein said list of proteins further comprises at least one additional protein selected from Table 1. 217. The method of embodiment 208, comprising obtaining age information for said individual. 218. The method of embodiment 208, comprising obtaining gender information for said individual. 219. The method of embodiment 208, comprising obtaining age information and gender information for said individual. 220. The method of any one of embodiments 208 to 219, further comprising transmitting a report to a health practitioner of results of said detecting. 221. The method of any one of embodiments 208 to 219, further comprising diagnosing said individual as having an advanced adenoma status when said protein levels and age from said individual as a whole do not differ significantly from a reference panel information set corresponding to a known advanced adenoma risk status. 222. The method of embodiment 220, wherein said report indicates recommendation for a colonoscopy for said individual. 223. The method of embodiment 220, wherein said report indicates recommendation for a polypectomy for said individual. 224. The method of embodiment 220, wherein said report indicates recommendation for radiation for said individual. 225. The method of embodiment 220, wherein said report indicates recommendation for chemotherapy for said individual. 226. The method of embodiment 220, wherein said report indicates recommendation for undergoing an independent cancer assay. 227. The method of embodiment 220, wherein said report indicates recommendation for undergoing a stool cancer assay. 228. The method of any one of embodiments 208 to 227, wherein said list of proteins comprises no more than 20 proteins. 229. The method of any one of embodiments 208 to 227, wherein said list of proteins comprises no more than 10 proteins. 230. A method of generating a biomarker panel for assessing a health status, comprising: a) identifying candidate biomarkers having an association with the health status; and b) performing mass spectrometric processing on at least a fragment of a plurality of candidate biomarker proteins derived from the candidate biomarkers to determine biomarkers suitable for assessing a health status; wherein the processing comprises at least one process control step. 231. The method of embodiment 230, wherein the at least one process control step comprises using at least one system suitability test (SST) run to assess liquid chromatography (LC) and mass spectrometry (MS) performance prior to the mass spectrometric processing. 232. The method of embodiment 231, wherein the SST comprises determining LC-MS performance by running a SIS standard curve in log-serial dilution. 233. The method of embodiment 232, further comprising performing a quality control check requiring at least about a 10-fold difference in MS signal between any two adjacent concentration levels, and a dynamic range of approximately four log units across the standard curve. 234. The method of embodiment 231, wherein the SST comprises determining LC performance by monitoring heavy transitions of internal standards for RT stability. 235. The method of embodiment 234, wherein monitoring heavy transitions comprises tracking RT shift between a detected value and a scheduled RT. 236. The method of embodiment 235, further comprising performing a quality control check requiring the upper 95% confidence interval of RTs of heavy transitions are no more than 6 seconds from the margins of LC-MS acquisition windows. 237. The method of embodiment 230, wherein the at least one process control step comprises monitoring flow-through AUC during immunodepletion, monitoring of TPA results for sample processing and immunodepletion efficiency, sample preparation customization depending on TPA result of each individual sample, or any combination thereof. 238. The method of embodiment 230, further comprising analyzing results of the mass spectrometric processing. 239. The method of embodiment 238, wherein the step of analyzing results comprises filtering transitions based on quantitative performance and peak quality. 240. The method of embodiment 239, wherein peak quality is evaluated using a peak quality tool. 241. The method of embodiment 230, wherein identifying candidate biomarkers comprises at least one of: obtaining biomarkers from an internal biomarker dataset, obtaining biomarkers from public biomarker datasets, or conducting a semi-automated literature search to identify biomarkers associated with the health condition. 242. The method of embodiment 241, wherein the step of analyzing results comprises requiring transitions to have labeled peaks in every processed sample. 243. The method of embodiment 230, wherein the at least one process control step comprises evaluating transitions for quantitative performance, peak quality, and the presence of labeled peaks in every processed sample. 244. The method of embodiment 230, wherein the at least one process control step comprises evaluating heavy and light transition pairs for at least one quantitative metric comprising heavy transition specificity, signal to noise ratio, precision, linearity, light transition specificity, or any combination thereof. 245. The method of any one of embodiments 230-244, further comprising evaluating only transitions that passed the at least one process control step. 246. A system for generating a biomarker panel for assessing a health status, comprising: a) a module identifying candidate biomarkers having an association with the health status; and b) a module performing mass spectrometric processing on at least a fragment of a plurality of candidate biomarker proteins derived from the candidate biomarkers to determine biomarkers suitable for assessing a health status; wherein the processing comprises at least one process control step. 247. The system of embodiment 246, wherein the at least one process control step comprises using at least one system suitability test (SST) run to assess liquid chromatography (LC) and mass spectrometry (MS) performance prior to the mass spectrometric processing. 248. The system of embodiment 247, wherein the SST comprises determining LC-MS performance by running a SIS standard curve in log-serial dilution. 249. The system of embodiment 248, further comprising performing a quality control check requiring at least about a 10-fold difference in MS signal between any two adjacent concentration levels, and a dynamic range of approximately four log units across the standard curve. 250. The system of embodiment 247, wherein the SST comprises determining LC performance by monitoring heavy transitions of internal standards for RT stability. 251. The system of embodiment 250, wherein monitoring heavy transitions comprises tracking RT shift between a detected value and a scheduled RT. 252. The system of embodiment 251, further comprising performing a quality control check requiring the upper 95% confidence interval of RTs of heavy transitions are no more than 6 seconds from the margins of LC-MS acquisition windows. 253. The system of embodiment 246, wherein the at least one process control step comprises monitoring flow-through AUC during immunodepletion, monitoring of TPA results for sample processing and immunodepletion efficiency, sample preparation customization depending on TPA result of each individual sample, or any combination thereof. 254. The system of embodiment 246, further comprising analyzing results of the mass spectrometric processing. 255. The system of embodiment 254, wherein the step of analyzing results comprises filtering transitions based on quantitative performance and peak quality. 256. The system of embodiment 255, wherein peak quality is evaluated using a peak quality tool. 257. The system of embodiment 246, wherein identifying candidate biomarkers comprises at least one of: obtaining biomarkers from an internal biomarker dataset, obtaining biomarkers from public biomarker datasets, or conducting a semi-automated literature search to identify biomarkers associated with the health condition. 258. The system of embodiment 257, wherein the step of analyzing results comprises requiring transitions to have labeled peaks in every processed sample. 259. The system of embodiment 246, wherein the at least one process control step comprises evaluating transitions for quantitative performance, peak quality, and the presence of labeled peaks in every processed sample. 260. The system of embodiment 246, wherein the at least one process control step comprises evaluating heavy and light transition pairs for at least one quantitative metric comprising heavy transition specificity, signal to noise ratio, precision, linearity, light transition specificity, or any combination thereof. 261. The system of any one of embodiments 246-260, wherein only transitions that passed the at least one process control step are evaluated to determine the biomarkers suitable for assessing health status. 262. A method of assessing a colorectal health risk status in an individual, comprising steps of: a) obtaining a circulating blood sample from said individual; and b) obtaining a biomarker panel level for at least two of A2GL, ALS, and PTPRJ of said circulating blood sample, and assessing colorectal health risk status. 263. The method of embodiment 262, wherein said biomarker panel further comprises an individual age. 264. The method of embodiment 262, wherein said colorectal cancer status comprises at least one of early CRC and advanced CRC. 265. The method of embodiment 262, wherein said colorectal cancer status comprises at least one of advanced adenoma, Stage 0 CRC, stage I CRC, Stage II CRC, stage III CRC, and stage IV CRC. 266. The method of embodiment 262, wherein said biomarker panel comprises no more than 20 proteins. 267. The method of embodiment 262, wherein said biomarker panel comprises no more than 10 proteins. 268. The method of embodiment 262, wherein said categorizing has a sensitivity of at least 70% and a specificity of at least 70%. 269. The method of embodiment 262, further comprising performing a treatment regimen in response to said categorizing. 270. The method of embodiment 269, wherein said treatment regimen comprises at least one of chemotherapy, radiation, immunotherapy, administration of a biologic therapeutic agent, polypectomy, partial colectomy, low anterior resection or abdominoperineal resection and colostomy. 271. The method of embodiment 262, further comprising transmitting a report of results of said categorizing to a health practitioner. 272. The method of embodiment 271, wherein said report indicates a sensitivity of at least 70%. 273. The method of embodiment 271, wherein said report indicates a specificity of at least 70%. 14. 274. The method of embodiment 271, wherein said report indicates a recommendation for a treatment regimen comprising at least one of chemotherapy, radiation, immunotherapy, administration of a biologic therapeutic agent, polypectomy, partial colectomy, low anterior resection or abdominoperineal resection and colostomy. 275. The method of embodiment 271, wherein said report indicates a recommendation for a colonoscopy. 276. The method of embodiment 271, wherein said report indicates a recommendation for undergoing an independent cancer assay. 277. The method of embodiment 271, wherein said report indicates a recommendation for undergoing a stool cancer assay. 278. The method of embodiment 262, further comprising performing a stool cancer assay in response to said categorizing. 279. The method of embodiment 262, further comprising continued monitoring for a period of 3 months or greater. 280. The method of embodiment 262, further comprising continued monitoring for a period of between 3 months and 24 months. 281. The method of embodiment 262, wherein said obtaining said protein levels comprises subjecting said biological sample to a mass spectrometric analysis. 282. The method of embodiment 281, wherein said mass spectrometric analysis is evaluated according to at least one process control step. 283. The method of embodiment 282, wherein the process control step comprises using at least one system suitability test (SST) run to assess liquid chromatography (LC) and mass spectrometry (MS) performance prior to the mass spectrometric processing. 284. The method of embodiment 262, wherein said obtaining said protein levels comprises subjecting said biological sample to an affinity assay. 285. The method of embodiment 284, wherein said affinity assay comprises an immunoassay analysis of said biological sample. 286. The method of embodiment 284, wherein said affinity assay comprises an aptamer analysis of said biological sample. 287. The method of embodiment 284, wherein said affinity assay comprises assessing said biological sample according to a quality control (QC) parameter. 288. The method of embodiment 287, wherein the QC parameter comprises at least one of sample integrity, sample elution efficiency, sample storage condition, and internal standard monitoring. 289. A method of generating a biomarker panel for assessing a health status, comprising: a) identifying candidate biomarkers having an association with the health status; and b) performing mass spectrometric processing on at least a fragment of a plurality of candidate biomarker proteins derived from the candidate biomarkers to determine biomarkers suitable for assessing a health status; wherein the processing comprises at least one process control step. 290. The method of embodiment 289, wherein the at least one process control step comprises using at least one system suitability test (SST) run to assess liquid chromatography (LC) and mass spectrometry (MS) performance prior to the mass spectrometric processing. 291. The method of embodiment 290, wherein the SST comprises determining LC-MS performance by running a SIS standard curve in log-serial dilution. 292. The method of embodiment 291, further comprising performing a quality control check requiring at least about a 10-fold difference in MS signal between any two adjacent concentration levels, and a dynamic range of approximately four log units across the standard curve. 293. The method of embodiment 289, wherein the SST comprises determining LC performance by monitoring heavy transitions of internal standards for RT stability. 294. The method of embodiment 293, wherein monitoring heavy transitions comprises tracking RT shift between a detected value and a scheduled RT. 295. The method of embodiment 292, further comprising performing a quality control check requiring the upper 95% confidence interval of RTs of heavy transitions are no more than 10% from the margin from the margins of LC-MS acquisition windows. 296. The method of embodiment 289, wherein the at least one process control step comprises monitoring flow-through AUC during immunodepletion, monitoring of TPA results for sample processing and immunodepletion efficiency, sample preparation customization depending on the TPA result of each individual sample, or any combination thereof 297. The method of embodiment 289, wherein the at least a fragment comprises a proteotypic peptide. 298. The method of embodiment 289, wherein the at least a fragment comprises a full length protein.

Further understanding of the disclosure herein is gained through reference to the following embodiments.

### EXAMPLES

### Example 1

A patient at risk of colorectal cancer is tested using a panel as disclosed herein. A blood sample is taken from the patient. The blood sample is mailed to a facility, where plasma is prepared and protein accumulation levels are measured using antibody florescence binding assay to detect members of a panel comprising A2GL, ALS, and PTPRJ, and also factoring in the patient's age. The patient's panel results are compared to panel results of known status, and the patient is categorized with an at least 81% sensitivity, and an at least 78% specificity as having colon cancer. A colonoscopy is recommended and evidence of colorectal cancer is detected in the individual.

### Example 2

The patient of Example 1 is prescribed a treatment regimen comprising a surgical intervention. A blood sample is taken from the patient prior to surgical intervention and protein accumulation levels are measured for a panel comprising A2GL, ALS, and PTPRJ, and also factoring in the patient's age. The patient's panel results are compared to panel results of known status, and the patient is categorized with an 81% sensitivity and a 78% specificity as having colon cancer.

A blood sample is taken from the patient subsequent to surgical intervention and protein accumulation levels are measured for a panel comprising A2GL, ALS, and PTPRJ, and also factoring in the patient's age. The patient's panel results are compared to panel results of known status, and the patient is patient is categorized with an 81% sensitivity, and a 78% specificity as having colon cancer.

### Example 3

The patient of Example 1 is prescribed a treatment regimen comprising a chemotherapeutic intervention comprising 5-FU administration. A blood sample is taken from the patient prior to chemotherapeutic intervention and protein accumulation levels are measured for a panel comprising A2GL, ALS, and PTPRJ, and also factoring in the patient's age. The patient's panel results are compared to panel results of known status, and the patient is patient is categorized with an 81% sensitivity, and a 78% specificity as having colon cancer.

A blood sample is taken from the patient at weekly intervals during chemotherapy treatment and protein accumulation levels are measured for a panel comprising A2GL, ALS, and PTPRJ, and also factoring in the patient's age. The patient's panel results are compared to panel results of known status. The patient's panel results over time indicate that the cancer has responded to the chemotherapy treatment and that the colorectal cancer is no longer detectable by completion of the treatment regimen.

### Example 4

The patient of Example 1 is prescribed a treatment regimen comprising a chemotherapeutic intervention comprising oral capecitabine administration. A blood sample is taken from the patient prior to chemotherapeutic intervention and protein accumulation levels are measured for a panel comprising A2GL, ALS, and PTPRJ, and also factoring in the patient's age. The patient's panel results are compared to panel results of known status, and the patient is patient is categorized with an 81% sensitivity, and a 78% specificity as having colon cancer.

A blood sample is taken from the patient at weekly intervals during chemotherapy treatment and protein accumulation levels are measured for a panel comprising A2GL, ALS, and PTPRJ, and also factoring in the patient's age. The patient's panel results over time indicate that the cancer has responded to the chemotherapy treatment and that the colorectal cancer is no longer detectable by completion of the treatment regimen.

### Example 5

The patient of Example 1 is prescribed a treatment regimen comprising a chemotherapeutic intervention comprising oral oxaliplatin administration. A blood sample is taken from the patient prior to chemotherapeutic intervention and protein accumulation levels are measured for a panel comprising A2GL, ALS, and PTPRJ, and also factoring in the patient's age. The patient's panel results are compared to panel results of known status, and the patient is patient is categorized with an 81% sensitivity, and a 78% specificity as having colon cancer.

A blood sample is taken from the patient at weekly intervals during chemotherapy treatment and protein accumulation levels are measured for a panel comprising A2GL, ALS, and PTPRJ, and also factoring in the patient's age. The patient's panel results are compared to panel results of known status. The patient's panel results over time indicate that the cancer has responded to the chemotherapy treatment and that the colorectal cancer is no longer detectable by completion of the treatment regimen.

### Example 6

The patient of Example 1 is prescribed a treatment regimen comprising a chemotherapeutic intervention comprising oral oxaliplatin administration in combination with bevacizumab. A blood sample is taken from the patient prior to chemotherapeutic intervention and protein accumulation levels are measured for a panel comprising A2GL, ALS, and PTPRJ, and also factoring in the patient's age. The patient's panel results are compared to panel results of known status, and the patient is patient is categorized with an 81% sensitivity, and a 78% specificity as having colon cancer.

A blood sample is taken from the patient at weekly intervals during chemotherapy treatment and protein accumulation levels are measured for a panel comprising A2GL, ALS, and PTPRJ, and also factoring in the patient's age. The patient's panel results are compared to panel results of known status. The patient's panel results over time indicate that the cancer has responded to the chemotherapy treatment and that the colorectal cancer is no longer detectable by completion of the treatment regimen.

### Example 7

A patient at risk of colorectal cancer is tested using a panel as disclosed herein. A blood sample is taken from the patient and protein accumulation levels are measured using reagents in an ELISA kit to detect members of a panel comprising A2GL, ALS, and PTPRJ, and also factoring in the patient's age. The patient's panel results are compared to panel results of known status, and the patient is patient is categorized with an 81% sensitivity, and a 78% specificity as having colon cancer. A colonoscopy is recommended and evidence of colorectal cancer is detected in the individual.

### Example 8

A patient at risk of colorectal cancer is tested using a panel as disclosed herein. A blood sample is taken from the patient and protein accumulation levels are measured using mass spectrometry to detect members of a panel comprising A2GL, ALS, and PTPRJ, and also factoring in the patient's age. The patient's panel results are compared to panel results of known status, and the patient is categorized with an 81% sensitivity, and a 78% specificity as having colon cancer. A colonoscopy is recommended and evidence of colorectal cancer is detected in the individual.

### Example 9

1000 patients at risk of colorectal cancer are tested using a panel as disclosed herein. A blood sample is taken from the patient and protein accumulation levels are measured to detect members of a panel comprising A2GL, ALS, and PTPRJ, and also factoring in the patient's age. The patients' panel results are compared to panel results of known status, and the patients are categorized with an 81% sensitivity, and a 78% specificity into a colon cancer category. A colonoscopy is recommended for patients categorized as positive. Of the patients categorized as having colon cancer, 80% are independently confirmed to have colon cancer. Of the patients categorized as not having colon cancer, 20% are later found to have colon cancer through an independent follow up test, confirmed via a colonoscopy.

### Example 10

A patient at risk of advanced adenoma is tested using a panel as disclosed herein. A blood sample is taken from the patient. The blood sample is mailed to a facility, where plasma is prepared and protein accumulation levels are measured using an antibody florescence binding assay to detect members of a panel comprising A2GL, ALS, and PTPRJ, and also factoring in the patient's age. The patient's panel results are compared to panel results of known status, and the patient is categorized as being at risk of advanced adenoma.

### Example 11—Identifying Protein Biomarkers

### Selection of Candidate Biomarkers

Candidate protein biomarkers can be selected from various sources. Examples of sources of candidate protein biomarkers include publicly available proteomics databases or datasets, internal datasets (e.g., from past internal studies), and scientific literature. The candidate protein biomarkers can be identified based on a known or inferred relationship with a disease or health status such as CRC. In some instances, the health status comprises the presence or absence of CRC. Alternatively or in combination, the health status comprises the grade or stage of CRC. Examples of CRC grades include low grade (e.g., the tumor has well differentiated cells that resemble normal cells and tend to be slower growing) and high grade (e.g., the tumor has poorly differentiated or undifferentiated cells that do not resemble normal cells and tend to be faster growing). In some cases, CRC grades include grade 0, grade 1, grade 2, grade 3, or grade 4. Grade 0 is the earliest stage of cancer and the tumor has not grown beyond the inner mucosal layer of the colon. Grades 1-4 are more advanced stages. In some cases, the systems and methods described herein enable detection of CRC that is grade 0, 1, 2, 3, or 4. Sometimes, the systems and methods enable detection of pre-CRC or increased risk of developing CRC that is even before grade 0. In some instances, candidate protein biomarkers for CRC are selected one or more of three sources: 1) an earlier targeted proteomics study performed in our laboratory, 2) analysis of publicly available proteomics datasets related to CRC, and 3) semi-automated literature searches. These three approaches yielded a total of 430 proteins designated as CRC-related biomarker candidates for further experimental investigation.

### List of Protein UniProt Entries for the 430 CRC-Related Biomarker Candidates

1433B_HUMAN; CH60_HUMAN; H2BFS_HUMAN; PCKGM_HUMAN; TNF15_HUMAN; 1433E_HUMAN; CHK1_HUMAN; HABP2_HUMAN; PDIA3_HUMAN; TNF6B_HUMAN; 1433F_HUMAN; CHK2_HUMAN; HEMO_HUMAN; PDIA6_HUMAN; TP4A3_HUMAN; 1433G_HUMAN; CHLE_HUMAN; HEP2_HUMAN; PDLI7_HUMAN; TPA_HUMAN; 1433T_HUMAN; CLC4D_HUMAN; HGF_HUMAN; PDXK_HUMAN; TPM2_HUMAN; 1433Z_HUMAN; CLUS_HUMAN; HMGB1_HUMAN; PEBP1_HUMAN; TR10B_HUMAN; 1A68_HUMAN; CNDP1_HUMAN; HNRPF_HUMAN; PEDF_HUMAN; TRAP1_HUMAN; A1AG1_HUMAN; CNN1_HUMAN; HNRPQ_HUMAN; PGFRA_HUMAN; TREM1_HUMAN; A1AG2_HUMAN; CO3_HUMAN; HPT_HUMAN; PIPNA_HUMAN; TRFE_HUMAN; A1AT_HUMAN; CO4A_HUMAN; HRG_HUMAN; PLGF_HUMAN; TRFL_HUMAN; A1BG_HUMAN; CO6A3_HUMAN; HS90B_HUMAN; PLIN2_HUMAN; TRI33_HUMAN; A2AP_HUMAN; CO8G_HUMAN; HSPB1_HUMAN; PLMN_HUMAN; TSG6_HUMAN; A2GL_HUMAN; C09_HUMAN; I10R1_HUMAN; PO2F1_HUMAN; TSP1_HUMAN; A2MG_HUMAN; COR1C_HUMAN; IBP2_HUMAN; PON1_HUMAN; TTHY_HUMAN; A4_HUMAN; CORIN_HUMAN; IBP3_HUMAN; POTEF_HUMAN; UGDH_HUMAN; AACT_HUMAN; CP1A1_HUMAN; IF4A3_HUMAN; PPIB_HUMAN; UGPA_HUMAN; ABCB5_HUMAN; CRDL2_HUMAN; IFT74_HUMAN; PRD16_HUMAN; UROK_HUMAN; ABCBA_HUMAN; CRP_HUMAN; IGF1_HUMAN; PRDX1_HUMAN; VCAM1_HUMAN; ACINU_HUMAN; CSF1_HUMAN; IGHA2_HUMAN; PRDX2_HUMAN; VEGFA_HUMAN; ACTBL_HUMAN; CSF1R_HUMAN; IGLL5_HUMAN; PREX2_HUMAN; VGFR1_HUMAN; ACTBM_HUMAN; CSPG2_HUMAN; IKKB_HUMAN; PRKN2_HUMAN; VILI_HUMAN; ACTG_HUMAN; CTHR1_HUMAN; IL23R_HUMAN; PRL_HUMAN; VIME_HUMAN; ACTH_HUMAN; CTNA1_HUMAN; IL26_HUMAN; PROC_HUMAN; VNN1_HUMAN; ADIPO_HUMAN; CTNB1_HUMAN; IL2RB_HUMAN; PROS_HUMAN; VP13B_HUMAN; ADT2_HUMAN; CUL1_HUMAN; IL6RA_HUMAN; PSME3_HUMAN; VTNC_HUMAN; AFAM_HUMAN; CYTC_HUMAN; IL8_HUMAN; PTEN_HUMAN; VWF_HUMAN; AGAP2_HUMAN; DAF_HUMAN; IL9_HUMAN; PTGDS_HUMAN; XBP1_HUMAN; AKA12_HUMAN; DEF1_HUMAN; ILEU_HUMAN; PTPRJ_HUMAN; ZA2G_HUMAN; AKT1_HUMAN; DESM_HUMAN; IPSP_HUMAN; PTPRT_HUMAN; ZMIZ1_HUMAN; AL1A1_HUMAN; DHRS2_HUMAN; IPYR_HUMAN; PTPRU_HUMAN; ZPI_HUMAN; AL1B1_HUMAN; DHSA_HUMAN; IRGM_HUMAN; PZP_HUMAN; ALBU_HUMAN; DPP10_HUMAN; ISK1_HUMAN; RAB38_HUMAN; ALDOA_HUMAN; DPP4_HUMAN; ITA6_HUMAN; RASF2_HUMAN; ALDR_HUMAN; DPYL2_HUMAN; ITA9_HUMAN; RASK_HUMAN; ALS_HUMAN; DYHC1_HUMAN; ITIH2_HUMAN; RBX1_HUMAN; AMPD1_HUMAN; ECH1_HUMAN; JAM3_HUMAN; RCAS1_HUMAN; AMPN_HUMAN; EDA_HUMAN; K1C19_HUMAN; REG4_HUMAN; AMY2B_HUMAN; EF2_HUMAN; K2C72_HUMAN; RET4_HUMAN; ANGI_HUMAN; ENOA_HUMAN; K2C73_HUMAN; RHOA_HUMAN; ANGL4_HUMAN; ENOX2_HUMAN; K2C8_HUMAN; RHOB_HUMAN; ANGT_HUMAN; ENPL_HUMAN; KAIN_HUMAN; RHOC_HUMAN; ANT3_HUMAN; ENPP1_HUMAN; KC1D_HUMAN; ROA1_HUMAN; ANXA1_HUMAN; ENPP2_HUMAN; KCRB_HUMAN; ROA2_HUMAN; ANXA3_HUMAN; EZRI_HUMAN; KISS1_HUMAN; RRBP1_HUMAN; ANXA4_HUMAN; FA10_HUMAN; KLK6_HUMAN; RSSA_HUMAN; ANXA5_HUMAN; FA5_HUMAN; KLOT_HUMAN; S100P_HUMAN; APC_HUMAN; FA7_HUMAN; KNG1_HUMAN; S10A8_HUMAN; APCD1_HUMAN; FA9_HUMAN; KPCD1_HUMAN; S10A9_HUMAN; APOA1_HUMAN; FABP5_HUMAN; KPYM_HUMAN; S10AB_HUMAN; APOA2_HUMAN; FAK1_HUMAN; LAMA2_HUMAN; S10AC_HUMAN; APOA4_HUMAN; FAK2_HUMAN; LAT1_HUMAN; S29A1_HUMAN; APOA5_HUMAN; FARP1_HUMAN; LBP_HUMAN; SAA1_HUMAN; APOC1_HUMAN; FBX4_HUMAN; LCAT_HUMAN; SAA2_HUMAN; APOC4_HUMAN; FCGBP_HUMAN; LDHA_HUMAN; SAA4_HUMAN; APOE_HUMAN; FCRL3_HUMAN; LEG2_HUMAN; SAHH_HUMAN; APOH_HUMAN; FCRL5_HUMAN; LEG3_HUMAN; SAMP_HUMAN; APOL1_HUMAN; FETA_HUMAN; LEG4_HUMAN; SBP1_HUMAN; APOM_HUMAN; FETUA_HUMAN; LEG8_HUMAN; SDCG3_HUMAN; ASAP3_HUMAN; FHL1_HUMAN; LEPR_HUMAN; SEGN_HUMAN; ATPB_HUMAN; FHR1_HUMAN; LEUK_HUMAN; SELPL_HUMAN; ATS13_HUMAN; FHR3_HUMAN; LG3BP_HUMAN; SEPP1_HUMAN; B2CL1_HUMAN; FIBA_HUMAN; LMNB1_HUMAN; SEPR_HUMAN; B2LA1_HUMAN; FIBB_HUMAN; LRRC7_HUMAN; SEPT9_HUMAN; B3GT5_HUMAN; FIBG_HUMAN; LUM_HUMAN; SF3B3_HUMAN; BANK1_HUMAN; FINC_HUMAN; LYNX1_HUMAN; SHIP1_HUMAN; BC11A_HUMAN; FLNA_HUMAN; LYSC_HUMAN; SHRPN_HUMAN; BCAR1_HUMAN; FLNB_HUMAN; MACF1_HUMAN; SIA8D_HUMAN; C1QBP_HUMAN; FLNC_HUMAN; MAP1S_HUMAN; SIAL_HUMAN; C4BPA_HUMAN; FND3B_HUMAN; MARE1_HUMAN; SIT1_HUMAN; CA195_HUMAN; FRIH_HUMAN; MASP1_HUMAN; SKP1_HUMAN; CAH1_HUMAN; FRIL_HUMAN; MASP2_HUMAN; SLAF1_HUMAN; CAH2_HUMAN; FRMD3_HUMAN; MBL2_HUMAN; SO1B3_HUMAN; CALR_HUMAN; FST_HUMAN; MCM4_HUMAN; SP110_HUMAN; CAPG_HUMAN; FUCO_HUMAN; MCR_HUMAN; SPB6_HUMAN; CASP9_HUMAN; FUCO2_HUMAN; MCRS1_HUMAN; SPON2_HUMAN; CATD_HUMAN; G3P_HUMAN; MIC1_HUMAN; SPP24_HUMAN; CATS_HUMAN; GAS6_HUMAN; MICA1_HUMAN; SRC_HUMAN; CATZ_HUMAN; GBRA1_HUMAN; MIF_HUMAN; SRPX2_HUMAN; CBG_HUMAN; GDF15_HUMAN; MMP2_HUMAN; STK11_HUMAN; CBPN_HUMAN; GDIR1_HUMAN; MMP7_HUMAN; SYDC_HUMAN; CBPQ_HUMAN; GELS_HUMAN; MMP9_HUMAN; SYG_HUMAN; CCD83_HUMAN; GFI1B_HUMAN; MTG16_HUMAN; SYNE1_HUMAN; CCL14_HUMAN; GGT1_HUMAN; MUC24_HUMAN; SYUG_HUMAN; CCR5_HUMAN; GHRL_HUMAN; MYL6_HUMAN; TACC1_HUMAN; CD109_HUMAN; GPNMB_HUMAN; MYL9_HUMAN; TAL1_HUMAN; CD20_HUMAN; GPX3_HUMAN; MYO9B_HUMAN; TBB1_HUMAN; CD24_HUMAN; GREM1_HUMAN; NDKA_HUMAN; TCTP_HUMAN; CD248_HUMAN; GRM6_HUMAN; NDRG1_HUMAN; TETN_HUMAN; CD28_HUMAN; GRP75_HUMAN; NFAC1_HUMAN; TF7L1_HUMAN; CD63_HUMAN; GSHR_HUMAN; NGAL_HUMAN; TFR1_HUMAN; CDD_HUMAN; GSTP1_HUMAN; NIBL2_HUMAN; THBG_HUMAN; CEA_HUMAN; GUC2A_HUMAN; NIPBL_HUMAN; THIO_HUMAN; CEAM3_HUMAN; H13_HUMAN; NNMT_HUMAN; THRB_HUMAN; CEAM5_HUMAN; H2A1D_HUMAN; NOD2_HUMAN; THTR_HUMAN; CEAM6_HUMAN; H2A2B_HUMAN; NUPR1_HUMAN; TIE2_HUMAN; CERU_HUMAN; H2AX_HUMAN; OSTP_HUMAN; TIMP1_HUMAN; CFAH_HUMAN; H2B1A_HUMAN; P53_HUMAN; TIMP2_HUMAN; CFAI_HUMAN; H2B1L_HUMAN; PAFA_HUMAN; TKT_HUMAN; CGHB_HUMAN; H2B1O_HUMAN; PAI1_HUMAN; TMG4_HUMAN; CH3L1_HUMAN; H2B3B_HUMAN; PALLD_HUMAN; TNF13_HUMAN;

Protein Biomarkers from an Earlier Study

An earlier targeted proteomics study focused on measuring 187 CRC-related proteins in 274 samples. All of these proteins were translated to the current project. Fresh method development was performed to find transitions that operated well in the complete method.

Protein Biomarkers from Analysis of Public CRC Datasets

Two publicly available proteomics datasets were obtained from the Clinical Proteomic Tumor Analysis Consortium (CPTAC) (https://cptac-data-portal.georgetown.edu/cptac/public). One offered shotgun proteomics measures from 95 CRC tumor samples analyzed earlier by The Cancer Genome Atlas (TCGA) (https://cptac-data-portal.georgetown.edu/cptac/s/S016, accessed August 2014). The second offered shotgun proteomics measures from normal colon tissue taken from 30 CRC patients (https://cptac-data-portal.georgetown.edu/cptac/s/S019, accessed August 2014). Both datasets originated from the same Proteome Characterization Center (Vanderbilt University), and were acquired using data-dependent MS2 methods on an LTQ Orbitrap Velos mass spectrometer. The datasets included relative abundance calculations for precursors and peptide sequence proposals based on MS2 spectra interpretation from database searching. Features with identical peptide sequence proposals were compared across the two datasets to find those that were significantly different using Student's t-test between normal and CRC tumor tissue. Any features found to be significantly different were then examined further to find those with peptide sequences uniquely linking them to a single protein. This procedure yielded 72 new candidate CRC-related proteins.

Protein Biomarkers from Semi-Automated Literature Searches

Semi-automated literature searches looked for co-occurrences of particular text terms in full-text PubMed Central (PMC, https://www.ncbi.nlm.nih.gov/pmc/) Open Access Subset and in PubMed abstracts. PubMed abstracts were searched for co-occurrences of common terms for CRC and of UniProt protein names and symbols, yielding 120 CRC-related proteins not used in the previous study. PMC open access articles were searched for co-occurrences of synonyms for “human”, “colon”, “cancer”, “plasma” or “serum”, and “protein”. Articles with these terms were additionally investigated to find any occurrences of UniProt protein names or symbols. The proteins were ranked by their number of mentions, and those proteins with the highest mention counts covering 95% of the total mentions were selected as candidate CRC-related proteins. This procedure yielded 172 new candidate CRC-related proteins.

### Selection of Proteotypic Peptides

The peptide selection process was performed using algorithms developed for the previous study and followed the guidelines established in published MS standards. Following in silico digestion of the proteins by trypsin, proteotypic peptides favoring zero miscleavage were selected for each protein by removing homologous peptides identified via BLAST sequence analysis. Next, some peptides were excluded because they have poor LC-MS responsiveness predicted by in silico models or include cysteine and methionine residues prone to chemical modification. The remaining peptides were then filtered by length, retaining those with 6-21 amino acids to ensure effective ionization and fragmentation. After these filtering steps, 1006 candidate proteotypic peptides covered the 431 proteins, with at least two peptides per protein.

### LC-dMRM/MS Optimization

The LC gradient was optimized by exploring LC gradient programs across repeated runs of a heavy peptide working solution. The working solution was a mix of stable isotope-labeled internal standards (SIS) (New England Peptide, Gardner, MA) consisting of nitrogen (15N) and carbon (13C) labeled versions (>95% purity) of the 1006 peptides with equal molar concentrations at 158 fmol/μL. Multiple reverse-phase chromatographic conditions were tested on a 1290 Infinity ultra-high performance liquid chromatography (UHPLC) system (Agilent Technologies) coupled with a 6550 quadrupole time-of-flight (Q-TOF) mass spectrometer (Agilent Technologies). Chromatographic separation was performed on a C18 column (Waters ACQUITY UPLC CSH, 2.1×150 mm, 1.7 μm particle size) with mobile phase A: 0.1% formic acid in water, and mobile phase B: 0.1% formic acid in acetonitrile. MS/MS spectra were acquired for heavy peptides exclusively and searched using in-house developed software for peptide identification and retention time assignment. The optimal LC gradient was established as that with the lowest gradient duration of less than 32 minutes, and with peptide concurrency approximately equal to 25 at any point, using an acquisition window of 42 sec and a cycle time of 500 ms. The final LC gradient used a flow rate of 450 μL/min on a 31.75 min linear gradient with the following segments: mobile phase B increased from 3% to 13% in the first 20 min, 13% to 20% in the next 7 min, 20% to 40% in the next 2 min, 40% to 80% in the next 1.25 min, and then stayed at 80% for the next 1.25 min before returning to 3% in the final 0.25 min.

With the final LC gradient, RTs were determined for 979 out of 1006 heavy peptides (430 out of 431 initial proteins). Skyline software (version 3.5) was used to list all possible singly charged product ion transitions for doubly charged precursor ions of the 979 peptides. From these ions, co-eluted ions with <=1 Da Mass difference were removed, leaving 12733 heavy transitions. From these 12733 transitions, small product ions b1, b2, y1, and y2 were excluded due to the risk of interference. The collision energy (CE) was then empirically optimized for the 8806 transitions using the heavy peptide working solution on a 1290 UHPLC coupled to a 6490 triple quadrupole (QQQ) mass spectrometer (Agilent Technologies). The CE calculated by Skyline software was used as a median value for CE optimization. CE optimization parameters were set to use 3 steps on each side of the value that was predicted by the default CE equation for each transition (CE=0.031 m/z+1), specified for Agilent QQQ mass spectrometer with the step size set to 6 V. In total, 6 collision energy voltage values were considered for each transition. The peak area under the curve (AUC) was integrated and analyzed with proprietary automated algorithms, developed at Applied Proteomics Inc. The CE that yielded the maximum peak AUC mean across 3 replicates was chosen as the optimal CE. A dynamic multiple reaction monitoring (dMRM) approach was selected for CE optimization and further experiments since it offers several advantages over the conventional segment dMRM approach for complex samples with low levels of the analytes of interest. The dMRM algorithm on the Agilent 6490 QQQ automatically constructed dMRM timetables throughout the LC-MS analysis based on the analyte RTs and acquisition windows. This approach allowed the instrument to acquire data only during specific RT windows, thus maximizing the concurrent ion transitions without compromising dwell time and sensitivity. The following conditions were maintained to ensure good signal to noise and sufficient data points across the peak of each transition based on our previous experience: acquisition window=42 seconds, dwell time>=2 ms, transition concurrency<=100, cycle time<=500 ms.

### Transition Screening

The 8806 transitions represented 901 proteotypic peptides from 430 proteins. The next step was to filter these to achieve acceptable LC concurrency and quality signal, aiming for two peptides/protein and two transitions/peptide. To this end, the transitions were first ranked and filtered according to five quantitative criteria related to heavy transition specificity, endogenous transition specificity, signal/noise, precision, and linearity. To obtain the five metrics, dMRM runs were performed using two 3-point curves of a heavy peptide mixture (15.8, 50, and 158 fmol/μL) in solvent and in endogenous matrix. For the solvent curve, the heavy peptide working solution was serially diluted in the half-log scale with the LC mobile phase (0.1% formic acid in 3% acetonitrile and 97% water). For the matrix curve, BioRec plasma was immuno-depleted and digested into endogenous peptides, and these lyophilized peptides were reconstituted to 3 μg/μL in each of the above three heavy peptide solutions. SIS curves in solvent and matrix were run in three technical replicates.

Transition specificity was evaluated by using the peak AUC ratio between two transitions of the same precursor (doubly charged peptide in this paper), referred to as “branching ratio” or “relative ratio”. The triplicate ratios were considered for all the transitions of each peptide. Heavy transition specificity was determined by a t-test comparing the heavy transition ratios in heavy peptide mixture (158 fmol/μL) with and without endogenous matrix. To evaluate light transition specificity, the acceptance requirement prior to performing the t-test was that heavy and light transition peaks co-elute with <=1-second difference between peak apexes, and then the comparison was performed between the transition ratios of heavy peptide and its corresponding light peptide in endogenous matrix spiked with heavy peptide solution at 158 fmol/μL. A p-value of 0.05 after multiple-test correction was the threshold to pass transition specificity and accept lack of interference. To evaluate signal/noise for each of the 8806 heavy transitions, averaged peak abundance was compared with instrument limit of quantitation (LOQ, 10× standard deviation of solvent blank's signal+averaged blank's signal) for each concentration level in the 3-point curve of the heavy peptide mixture in solvent. Signal abundance at 50 fmol/μL must be above or equal to instrument LOQ for the transition to pass the criterion of signal/noise. Precision was measured with the triplicate 3-point curves of the heavy peptide mixture (15.8, 50, and 158 fmol/μL) in solvent. Coefficient of variation (CV) was calculated for peak AUCs of heavy transition between three repeats at each concentration level. Three peak AUC values were required for all three dilution steps with CVs <=20% for the transition to pass the metric of precision. Linearity was assessed with a linear regression applied across the three concentration levels. The criteria for acceptance were that the multiple-test corrected p-value for slope must be <0.05, that the slope must be >0, and that the slope confidence interval must exclude 0.

Following the above measurements and calculations, each transition had a binary pass/fail result for each of five metrics and was assigned to one of ten tiers based on the combination of the five binary results in the hierarchical order of heavy transition specificity, signal/noise, precision, linearity, and light transition specificity as shown in Table 3.

TABLE 3
10-Tier System For Transition Ranking And Filtering

| | Heavy | | | | Light |
|---|---|---|---|---|---|
| | Transition | | | | Transition |
| Tier | Specificity | Signal/Noise | Precision | Linearity | Specificity |
| 1 | Pass | Pass | Pass | Pass | Pass |
| 2 | Pass | Pass | Pass | Pass | Fail |
| 3 | Fail in any one criterion | | | | |
| 4 | Pass | Pass | Pass | Fail | Fail |
| 5 | Fail in any two criteria | | | | |
| 6 | Pass | Pass | Fail | Fail | Fail |
| 7 | Fail in any three criteria | | | | |
| 8 | Pass | Fail | Fail | Fail | Fail |
| 9 | Fail in any four criteria | | | | |
| 10 | Fail | Fail | Fail | Fail | Fail |

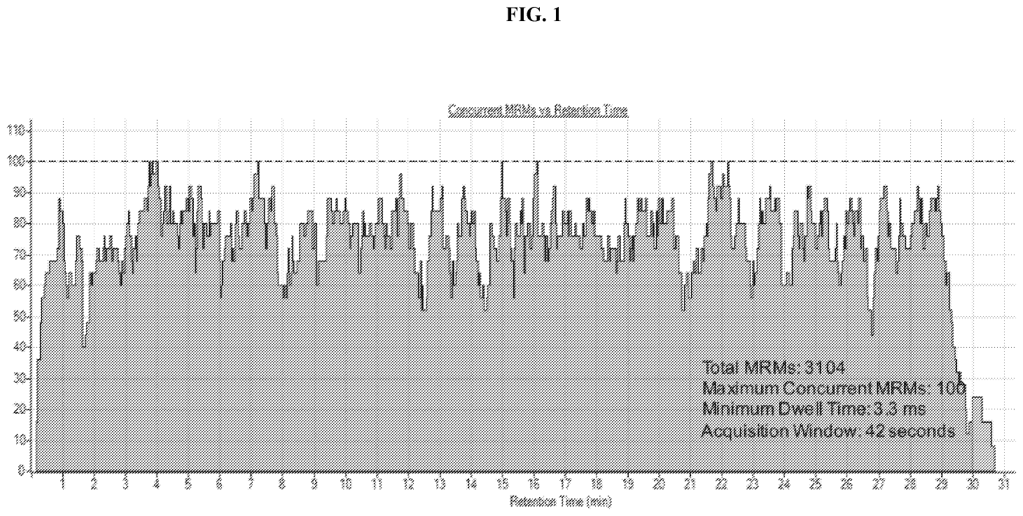

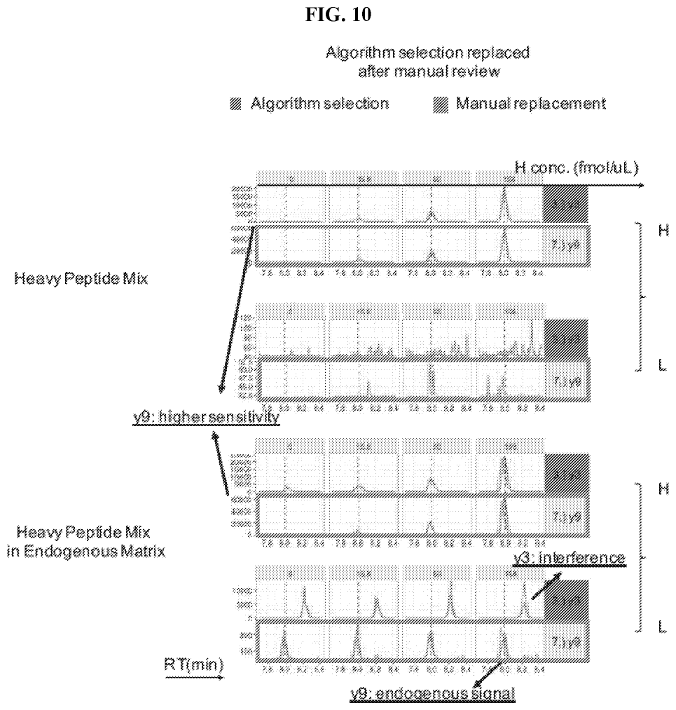

All 8806 transitions were automatically ranked in this novel 10-tier system. In the event of multiple transitions from a given peptide assigned to the same tier, the transition peak AUC was used as tiebreaker, such that the transition with the higher AUC would be ranked higher. Transitions were then selected by a proprietary automated algorithm with transitions from tiers 1 and 2 selected as first choice to increase assay quality, followed by a secondary transition selection from the other tiers to increase assay quantity while maximizing protein number in the final dMRM assay. Overall, one (required) to two (preferred) top-ranked peptides were chosen for each protein, and at least two top-tier transitions were picked for each peptide. These two transitions might be used in later analyses as a quantifier and a qualifier, conforming to some recommended analysis procedures. An output report was generated from the proprietary algorithm for a manual review to confirm the transition performances and selections. A minimal manual replacement was performed for the cases shown in FIG. 10. Ultimately, the final dMRM method, summarized in Table 4, included 1552 high-quality transitions (3104 heavy & light transitions) selected for 641 peptides representing 392 CRC proteins while transition concurrency was capped at 100 transitions for every 42-second LC-MS acquisition window as demonstrated in FIG. 1. FIG. 1 shows a first shading starting from around 0 minutes retention time on the x-axis and ending at about 30 minutes. A second, lighter shading begins at around 30 minutes and ends before 31 minutes.

TABLE 4
Summary Of Final MRM Method
The Final LC-MRM Method

| | |
|---|---|
| LC Gradient (min) | 31.75 |
| # Proteins | 392 |
| # Peptides | 641 |
| # Transition Pairs (Heavy + Light) | 1552 (3104) |
| # Peptides with 2 Transition Pairs | 79% (506/641) |
| # Peptides with > 2 Transition Pairs | 21% (135/641) |
| # Proteins with Only 1 Peptide | 37% (146/392) |
| # Proteins with 2 or More Peptides | 63% (246/392) |

Analytical Performance of the Final dMRM Method

Transition analytical performance in the final method was characterized next. This process used a new heavy peptide solution consisting of the final 641 SIS peptides with equal molar concentrations at 500 fmol/μL. This mixture was diluted to give a 10-point half-log-serial dilution series with concentrations of 0.0158, 0.05, 0.158, 0.5, 1.58, 5, 15.8, 50, 158, and 500 fmol/μL. 100 μL aliquots of each heavy peptide dilution were added to 300 μg of lyophilized endogenous peptides processed from BioRec plasma to give the standard series. In addition, one plasma matrix preparation was reconstituted with solvent to serve as a blank. Standards and blanks were run in triplicate on one instrument (Agilent 1290 UHPLC-6490 QQQ) over one day. Plate- and sample-level quality metrics were assessed as described below for study runs; no quality failures were encountered.

Sensitivity assessments began by determining the Limits of Blank (LoB) and Limits of Detection (LoD) for each of the 1552 heavy transitions. These were determined by using triplicate means and standard deviations to estimate percentiles that reasonably define the LoB and LoD. Specifically, the LoB was defined as the estimate of the 95th percentile of heavy transition peak area in the blank, and the LoD was defined as the minimum standard concentration at which the estimate of the heavy transition peak area's 5th percentile was greater than or equal to the LoB. Assuming normal distributions, the LoB and LoD were calculated as follows.

LoB=meanblank+(1.645×sdblank)

LoD=minimum standard concentration at which

meanstandard−(1.645×sdstandard)>=LoB

Linearity assessments consisted of finding the largest set of standards that met pre-specified criteria and that supported a linear response range for each of the 1552 heavy transitions. The criteria for standard measures to be included in linearity assessment were 1) CV<=30% and 2) nominal concentration>=LoD. Using these standards' measures for each heavy transition, a robust linear model was used to fit transition peak area to nominal standard concentration. If the fit slope's 95% confidence interval matched or extended below 0, the lowest standard concentration was dropped, and the fit was attempted again. This process was repeated until 1) fewer than three concentrations remained (linear fit failure), or 2) the fit slope's 95% confidence interval was positive and excluded 0 (linear fit success). Lower Limits of Quantitation (LLoQ), an additional sensitivity metric, were determined from the linearity assessments. For successful linear fits, the LLoQ was the nominal concentration of the lowest standard used in the fit.

Finally, the linear dynamic range of each heavy transition was calculated from the ratio of the maximum and minimum standard concentrations from a successful linear fit:

dynamic range=log 10(standard·concnmax/standard·concnmin)

All heavy and light transition pairs with successful linear fits (requiring a defined LoB, a defined LoD, at least 3 standard concentrations >=LoD and with CVs <=30%, and a positive linear slope distinguishable from 0) were considered to have quantitative performance.

### Biomarker Study Implementation and Performance Monitoring

The principal variables influencing the precision and accuracy of an dMRM-based quantitative experiment are often related to either the pre-analytical or analytical aspects of the study. In this study, the pre-analytical variables—sample-specific differences in collection, processing, handling and storage procedures—were controlled by implementing standard operating procedures (SOPs) during collection of the Endoscopy II specimens. In one aspect of this disclosure, we address analytical variation and review the procedures we have used to monitor the analytical variability in a large-scale, longitudinal study using multiple instruments over four months. The quality parameters we monitor address the sample processing, LC performance, MS performance, or any combination thereof.

### Patient Samples

The patient samples used in this study were drawn from a high-quality clinical sample set, Endoscopy II, described previously. In brief, plasma samples were collected between 2010 and 2012 at seven hospitals in Denmark from patients considered high risk for CRC because of symptoms of colorectal neoplasia. The study inclusion criteria encompassed age≥18 years, scheduled for first-time colonoscopy, and any symptom of colorectal neoplasia (abnormal bowel habits, abdominal pain, rectal bleeding, unexplained weight loss, meteorism, anemia, and/or palpable mass). Colonoscopies, which followed sample collection, revealed the presence or absence of CRC, with CRC staged according to the Union for International Cancer Control (UICC) tumor node metastasis (TNM) system. Each Endoscopy II patient was placed in one of eight diagnostic groups based on colonoscopy results and comorbidities: colon cancer (all stages), rectal cancer (all stages), colon adenoma, rectal adenoma, no comorbidities and no CRC or polyps (“no comorbidity-no finding” group), comorbidities present and no CRC or polyps (“comorbidity-no finding” group), other cancer(s), or other colonoscopy findings (“other findings”). Comorbidity referred to co-existing medical ailments not related to CRC, such as Crohn's disease, colitis, diverticulitis, acute chronic inflammation, diabetes, rheumatoid arthritis, cardiovascular diseases, cirrhotic liver diseases, obstructive lung diseases, or restrictive lung diseases. A total of 1045 Endoscopy II plasma samples was used in this biomarker discovery study. The distribution of the 1045 patient samples across the diagnostic groups is presented in Table 5.

TABLE 5
Patient Sample Distribution

| | | Discovery Set: | Test Set: | |
|---|---|---|---|---|
| | | Enriched for | Intent-to- | |
| | | CRC & | Test | |
| Patient Diagnostic Groups | | Adenoma | Proportions | Total |
| Cases | Colon Cancer | 134 | 26 | 160 |
| | Rectal Cancer | 82 | 16 | 98 |
| Controls | Colon Adenoma | 127 | 41 | 168 |
| | Rectal Adenoma | 51 | 14 | 65 |
| | Other Cancer | 14 | 14 | 28 |
| | Other Finding | 106 | 106 | 212 |
| | Comorbidity—No | 65 | 64 | 129 |
| | Finding | | | |
| | No Comorbidity—No | 93 | 92 | 185 |
| | Finding | | | |
| Total | | 672 | 373 | 1045 |

The 1045 patients were divided into separate Discovery and Validation (Test) sets, consisting of 672 and 373 patients, respectively. Data from the Discovery set were used to provide an overview of CRC signal as evidenced by univariate measures. Data from the Validation set were not analyzed in the current study; these data were retained for future validation/testing following multivariate classifier development.

### LC-MS Sample Processing and Performance Monitoring

Plasma samples were visually inspected to exclude lipemic and hemolytic samples. They were then processed into lyophilized protein digests as previously described. Briefly, a single 25 μL plasma aliquot from each sample was filtered to remove lipids and loaded on a 10 mm×100 mm Human 14 MAR column (Agilent Technologies) for immuno-depletion. The flow-through fractions, representing depleted plasma, were collected for buffer exchange with ammonium bicarbonate before protein concentration determination (Quant-iT Protein Assay Kit, ThermoFisher Scientific) performed on a Freedom EVO 200 automated liquid handling system (Tecan), used as the total protein assay (TPA) result. The TPA result for each sample was used to determine the amount of enzyme to be added during protein digestion (trypsin to protein mass ratio=1:34), and also to calculate the volume of LC-MS sample reconstitution solution aiming for 3 μg/μL of endogenous protein concentration, prior to LC-MS analysis. Protein digestion on a Freedom EVO 150 platform (Tecan) started with protein denaturation with 2,2,2-trifluoroethanol (Acros), followed by reduction with DL-dithiothreitol (Sigma-Aldrich) and subsequent alkylation with iodoacetamide (Arcos). Appropriate trypsin (Promega) was added into each sample before the incubation at 37° C. for 16 hours. The reaction was stopped with 10 μL of neat formic acid (ThermoFisher Scientific), followed by lyophilization. Prior to LC-MS injection, each endogenous sample was reconstituted in the appropriate volume of heavy peptide solution (SIS mixture with equal molar concentration at 100 fmol/μL) to get 30 μg of endogenous protein and 1,000 fmol of each heavy peptide in a single injection (10 μL) loaded onto the LC column.

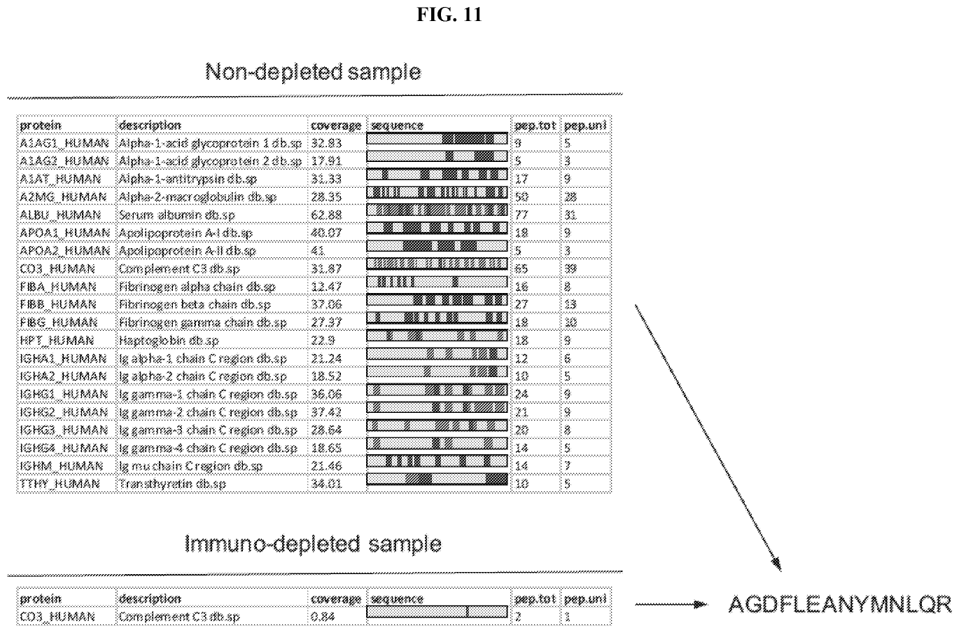

Laboratory automation was deployed for the TPA procedure, protein digestion, and LC-MS sample reconstitution to ensure operation reproducibility by eliminating error-prone manual procedures with automated processes requiring minimal technician involvement. Immuno-depletion efficiency was pretested with two aliquots of 25 μL BioRec plasma being processed with and without the step of immuno-depletion respectively. 91% (1365 μg/1500 μg) proteins were depleted based on TPA results and only one peptide of Human 14 proteins was detected in the depleted flow-through collection by LC-MS/MS (FIG. 11). As shown in FIG. 11, the shaded sections of the sequence correspond to peptides in the sample (before and after immune-depletion, respectively). For the one detected peptide: Complement C3_AGDFLEANYMNLQR, MS1 EIC peak area is 1% of that measured in the same peptide from the non-depleted sample while LC-MS injection load was 30 μg for both samples.

The 1045 patient samples were randomized and divided into 66 batches of up to 16 samples each. Each batch also included four aliquots of a pooled set of plasma samples (BioReclamationTVT), referred to as process quality controls (PQCs). Two batches were run each day—one on each of two immuno-depletion systems coupled with two LC-MS workstations. Reproducibility of the sample processing was evaluated over the four-month study period. The UV (220 nm) chromatograms in protein depletion were overlaid daily for each batch to review every PQC and patient sample, with the reference of the runs in the study day 1 and the previous day to check uniformity of peak shape and RT. PQCs' flow-through peak AUCs in the step of immuno-depletion and TPA results were tracked and compared with the ranges of means+/−standard deviations. After processing each batch, one of the four PQCs was analyzed by full MS and tandem MS to further monitor immuno-depletion and trypsin digestion. Immuno-depletion efficiency was evaluated by investigating the presence or absence of the top 14 human plasma proteins. Digestion consistency was assessed by monitoring the counts of molecular features (z at 2-4) detected by full MS and the missed cleavage rate in MS2 data search.

### LC-MS Data Acquisition, Reduction, and Performance Monitoring

The biomarker study was run using the optimized LC gradient and the final dMRM method on two sets of 1290 UHPLC coupled to 6490 QQQ (Agilent Technologies). Both 6490 QQQs were operated in positive mode and ionization source conditions were as follows: capillary voltage=3.5 kV, nozzle voltage=300 V, nebulizer pressure=20 psi, sheath gas flow=11 L/min and sheath gas temperature=250° C. Each LC-MS worklist was comprised of an initial 5-point standard curve of 641 heavy peptides in solvent (0.05-500 fmol/μL, log serial dilution), 3 PQCs at the beginning, middle and end of the run, 16 individual patient samples, and 7 Blank samples (LC solvent) interspersed throughout the worklist to evaluate carryover. One single injection per sample was loaded on LC-MS for 40-minute data collection and the entire worklist required 21 hours. The study took four months to complete data collection using two LC-MS workstations, with instrument maintenance performed daily to ensure consistent LC-MS performance.

MS raw data were automatically extracted, reduced, and integrated, and then visualized using a real-time analytical pipeline developed at Applied Proteomics, Inc. An internal web client, accessing the pipeline server, permitted monitoring of data reduction, reviewing dMRM traces for each targeted transition, and downloading data for further analyses. Additionally, R scripts were created specifically to consolidate processed data and automate LC-MS performance monitoring. The LC-MS system suitability test (SST) and LC-MS performance during data acquisition were monitored using reference materials consisting of processed PQC samples and heavy peptide solution (mix of the final 641 SIS peptides with equal molar concentrations at 500 fmol/μL).

Immediately prior to each of the sample batch runs, the SST was performed to determine LC-MS performance by running the 5-point SIS standard curve in log-serial dilution. LC performance was checked by monitoring all 1552 heavy transitions (internal standards) for RT stability. An RT plot was automatically generated for each data file immediately after it was processed through the pipeline, tracking RT shift between the detected value and the scheduled RT used in the method. In order to avoid truncated peaks, the main quality control check required that the upper 95% confidence interval of the 1552 heavy transitions' RTs were <=6 seconds from the margins of LC-MS acquisition windows. If this check failed, troubleshooting followed by RT reassignment if necessary was performed before further data acquisition. MS performance was checked using 176 high performing heavy and light transition pairs that were selected during assay development to serve as QC transitions. In the SST, peak AUCs were recorded for the heavy QC transitions across the five concentration levels on the SST 5-point standard curves. The main quality control check required an approximately 10-fold difference in MS signal between any two adjacent concentration levels, and a dynamic range of approximately four log units across the full curve. If this check failed, troubleshooting was performed before further data acquisition. For each standard concentration, heavy transition peak AUCs were compared across days and between LC-MS systems to determine consistent MS performance across the four-month data collection period.

The sample batch set-up was leveraged to evaluate the performance of each LC-MS system during data acquisition and to establish confidence in the quality of the acquired sample measurements. This was accomplished by analyzing data from the PQCs at the beginning, middle and end of each worklist, thereby providing information on the daily performance of each of the LC-MS systems during the experimental runs. The PQCs enabled LC-MS monitoring using both signal intensity and retention time stability. Heavy and light peak AUCs were tracked for the 176 QC transition pairs in PQC samples to confirm MS performance. CVs were calculated across three PQCs in each batch to evaluate intra-batch precision. Individual PQC plots were generated daily for both heavy and light peaks of the QC transitions to demonstrate peak AUC and CV trends over the four months. In addition, RT plots tracking RT shifts of 1552 heavy transitions were generated for all the 1045-patient data files to confirm data quality.

### Study Sample Data Processing

Data were compiled for the labeled and light peaks for each of the 1552 transition pairs in the final dMRM method, across all 1045 patient samples of the study. Prior to evaluating CRC signal, transition pairs were evaluated along three quality metrics; only transitions that passed all three checks were used to assess CRC signal in the study.

First, transitions were evaluated as to their quantitative performance. Specifically, the standard curve for a transition pair's labeled peak was required to have a successful linear fit (requiring a defined LoB, a defined LoD, at least 3 standard concentrations >=LoD and with CVs <=30%, and a positive linear slope distinguishable from 0).

Second, transitions were required to have high quality peaks. Peak quality was assessed with a proprietary machine learning tool developed in-house. Instead of directly assessing peak shape itself, the in-house tool integrated information about several parameters that, together, were found to be strongly associated with clearly favorable (large and easily recognized) peak shapes. These parameters covered seven measures related to labeled peak area, the consistency of labeled peak area, light peak area, light/labeled peak ratios, the difference between labeled peak retention time and expected retention time, consistency of labeled peak retention times, and consistency of differences between labeled and light peak retention times. The tool validated with 95% accuracy in predicting manual assessments of peak quality.

Third, transitions were required to have labeled peak measured in all 1045 samples. In combination with the other two criteria, this ensured that signal measurement was valid in all samples, thus obviating any need for imputation.

For transitions that passed these three quality checks, the light peak's endogenous concentration in each sample was calculated as the ratio of light/heavy peak area multiplied by the known spike-in concentration of the heavy peak. These endogenous concentrations were used to calculate each transition's univariate CRC signal; receiver operating characteristic (ROC) analysis was used to calculate a CRC vs nonCRC AUC in the 672-sample Discovery set. ROC analysis was performed using the pROC package (version 1.10.0). In addition, statistical tests (Student's T Test, and the Wilcoxon Rank Sum Test) were run to evaluate whether each transition's concentration was significantly different between CRC and nonCRC samples in the Discovery set. All analyses were performed using the R programming language running in Unix and OSX environments.

### Results and Discussion

### Optimization of LC-dMRM/MS

We previously reported an LC-dMRM method that measured 337 peptides from 187 proteins with a 29-minute gradient on an LC-MS system of Agilent 1290 UHPLC-6490 QQQ. In this study, we developed a new expanded method, in which the LC gradient was further optimized to separate a new candidate list of 1006 peptides in 32 minutes on the same LC-MS workstation. In some cases, the optimal gradient program would have elution concurrency at or below 25 peptides in every 42-second acquisition window over the entire LC method. The final gradient program located RTs of 979 peptides representing 430 proteins and achieved this concurrency requirement for 63% of the 979 peptides across 82% of the entire 31.75-min LC gradient. In addition, the full width half maximum (FWHM) of heavy peptide MS1 EIC peaks centered around 5-6 seconds (median 5.5 seconds)—wide enough to obtain 15-20 data points across each peak using a 500 ms cycle time, and narrow enough to accommodate RT shifts in the 42-second acquisition window.

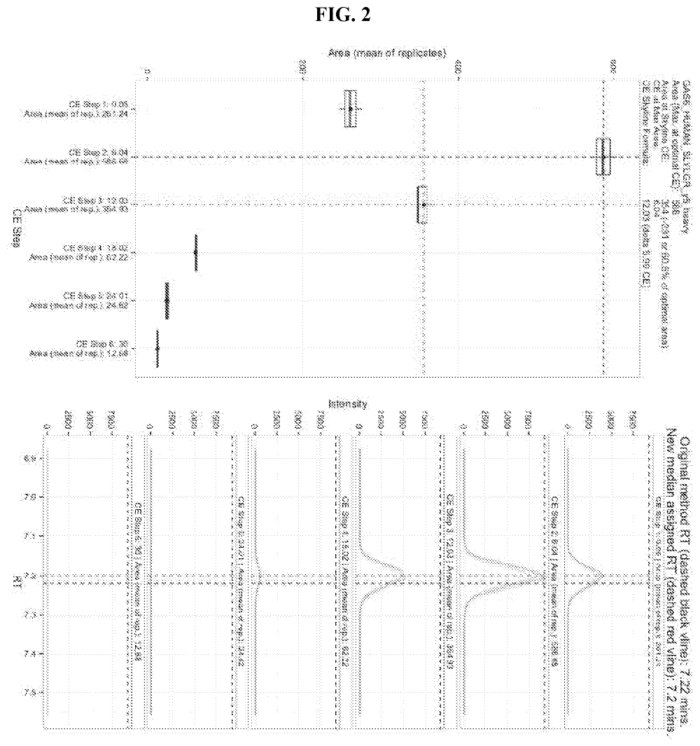

Following LC optimization, the optimal CE was empirically determined for each of the 8806 heavy transitions as the CE yielding the highest average labeled peak AUC. An example of CE optimization for the heavy transition SLYLGR→y5 is shown in FIG. 2. Both box plots and dMRM profiles demonstrated that the optimal CE of 6.04 V at step 2 generated the most abundant signal (average AUC=586.68; see right vertical dashed line and top horizontal dashed line and their intersection), 65% higher than the 2nd abundant signal obtained at CE step 3 predicted by Skyline (average AUC=354.93; see the left vertical dashed line and the bottom horizontal dashed line and their intersection). The box plot of RT vs intensity shows a dashed line for the original method at 7.22 minutes and a dashed line for the new median assigned RT at 7.2 minutes (slightly to the left of the dashed line for the original method) at each CE step.

Transition Selection to Build the Final Multiplexed dMRM Assay

With the optimal LC-MS condition, the 8806 heavy and light transition pairs were experimentally studied to select robust and interference-free transitions. Each transition pair was evaluated for passing or failing 5 quantitative criteria in the order of priority above. The passing rate in 8806 transitions for each of the five metrics is summarized in Table 6.

TABLE 6
Results Of Transition Filtering With Five Metrics

| Filtering Metrics for 8806 | # Transition Passing | Passing |
|---|---|---|
| Transitions | Each Metric | Rate |
| Heavy Transition Specificity | 6402 | 73% |
| Instrument LOQ | 8490 | 96% |
| Precision & Linearity | 5347 | 61% |
| Light Transition Specificity | 6710 | 76% |

Transitions were automatically categorized and selected using the 10-tier ranking system (Table 3) with a proprietary algorithm, resulting in 1552 top performing transition pairs selected to represent 641 peptides from 392 CRC proteins. In detail, 718 transitions from tiers 1 and 2 were first chosen for 359 peptides representing 183 proteins. To increase the proteins covered, a second transition selection was performed for the remaining 247 proteins. An additional 558 top-performing transitions were selected in all the tiers for 279 peptides representative of 209 proteins. Next the unselected transitions of the existing 392 proteins were backfilled for any 42-second acquisition windows with transition concurrency <90 until it was equal to 90. An additional top-ranked 276 transitions were added for 3 peptides in the final assay. Following the automatic selection, manual review was performed and 117 of 1552 transitions (7.5%) were manually replaced due to interference.

Our 10-tier transition ranking system, incorporating five quantitative criteria, used a strict cutoff for each criterion to select the highest quality targets suitable for inclusion in the final dMRM method. This automated process was found to be accurate when compared to a small-scale manual transition selection that was performed in parallel. In addition, the speed and objectivity of the automated process render it preferable to manual processes.

### Analytical Performance

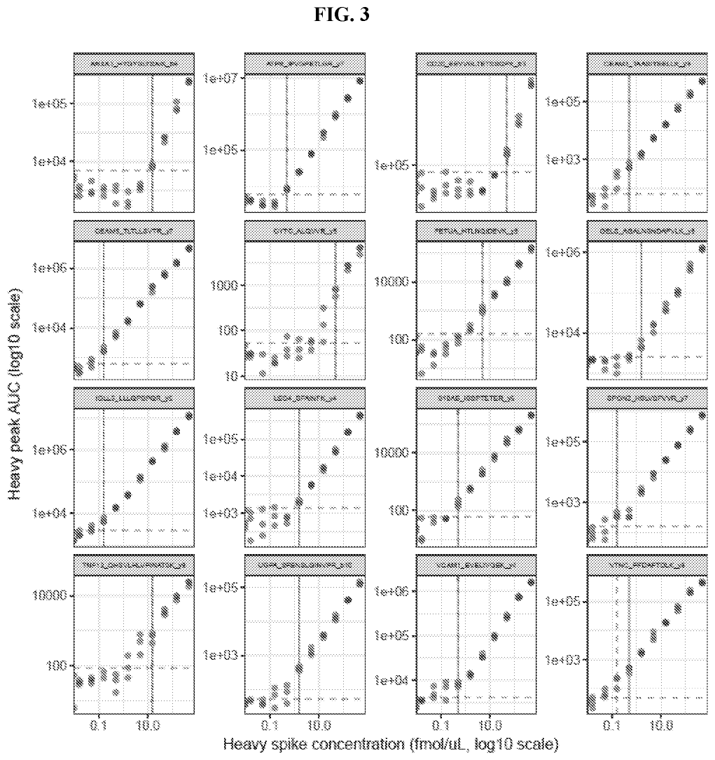

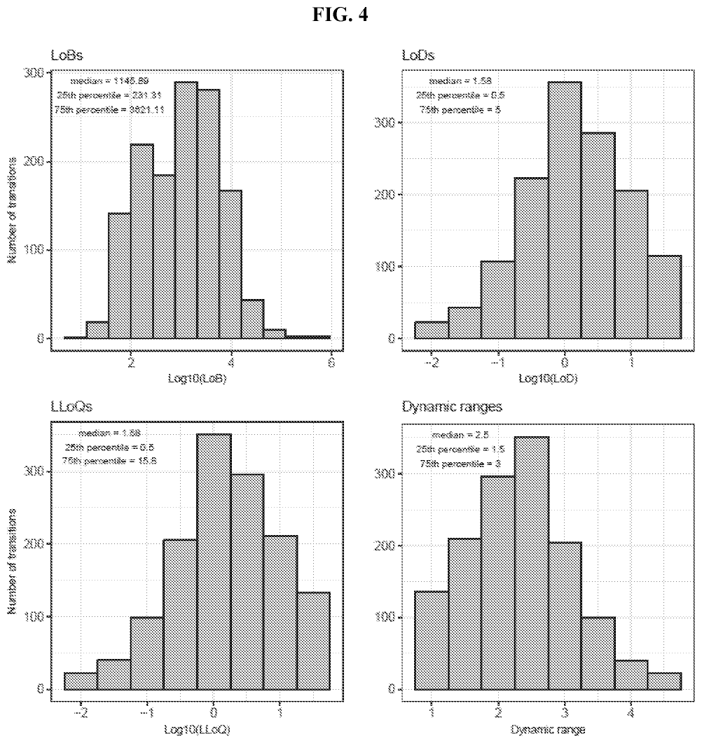

After method development, each transition's analytic performance was characterized by considering LoBs, LoDs, LLoQs, and dynamic ranges established on the basis of 10-point standard curves run using the finalized method. Of the 1552 total transitions, 1357 had valid measures for all of these metrics. Example standard curves are shown in FIG. 3. These examples illustrate the range of transition assays observed—LoBs, LoDs, LLoQs, and linear dynamic ranges all varied substantially. These examples also show that for many transitions, LoDs match LLoQs; for a few, such as that shown at the lower right, LLoQs were above LoDs. Each standard curve has lighter background vertical and horizontal lines, and a darker vertical line and a dashed horizontal line. To get a sense of how the metrics varied across all 1357 transitions, FIG. 4 offers frequency histograms and summary statistics for the metrics across the 1357 transitions.

The 1357 transitions for which analytical performance could be assessed covered 87.4% of the 1552 transitions measured in the study. On the peptide level, these 1357 transitions covered 596, or 93.0%, of the 641 peptides in the study. On the protein level, these 1357 transitions covered 373, or 95.2%, of the 392 proteins in the study.

### Monitoring Analytical Variability

Protein Immunodepletion and Digestion

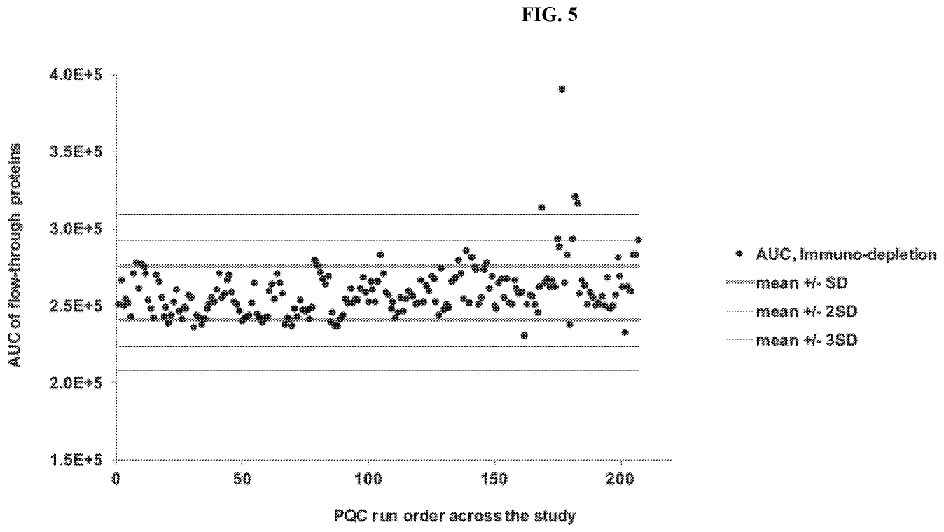

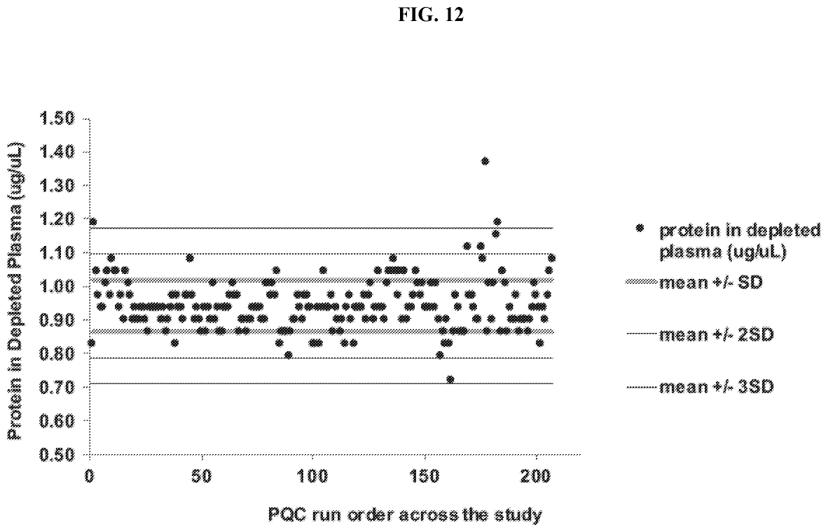

The reproducibility of sample analysis is dependent on the consistency of sample preparation prior to data collection. In this study, we evaluated two processing steps subject to sample variation: immuno-depletion and trypsin digestion. To assess the reproducibility of plasma immuno-depletion, a photodiode array (PDA) detector using ultraviolet detection (220 nm) monitored peak AUC and RT for both the flow-through and bound fractions. The consistency in immuno-depletion was observed by overlaying UV traces of samples within a run and between days. 207 PQCs' flow-through peak AUCs (depleted plasma fractions) were monitored over the four-month study period. FIG. 5 demonstrated that 98% PQCs have flow-through peak AUCs within the range of mean+/−3 standard deviations. One PQC was excluded from LC-MS data analysis due to high flow-through peak AUC far above mean+3 SD (bracketed by the highest and lowest solid lines shown on the graph) and caused by the swap of sample vial between the PQC and the adjacent sample. The mean+2 SD is bracketed by the solid lines to the inside of the +3 SD lines. The innermost two lines that are thicker than the +2 or +3 SD lines indicate the +1 SD. The sample redo was performed. The consistent immuno-depletion over time was also indicated by TPA results (FIG. 12). One PQC was excluded from LC-MS data analysis due to high flow-through peak AUC far above mean+3 SD (bracketed by the highest and lowest solid lines shown on the graph) and caused by the swap of sample vial between the PQC and the adjacent sample. The mean+2 SD is bracketed by the solid lines to the inside of the +3 SD lines. The innermost two lines that are thicker than the +2 or +3 SD lines indicate the +1 SD. Only 3 out of 207 PQCs have protein concentrations in depleted plasma large than mean+3 SD. The immuno-depletion efficiency was also calculated by TPA result. Immuno-depletion efficiency=1−mean of protein concentration in depleted plasma (0.94 μg/μL) divided by estimated protein concentration in regular plasma (75 μg/μL)=98.7%.

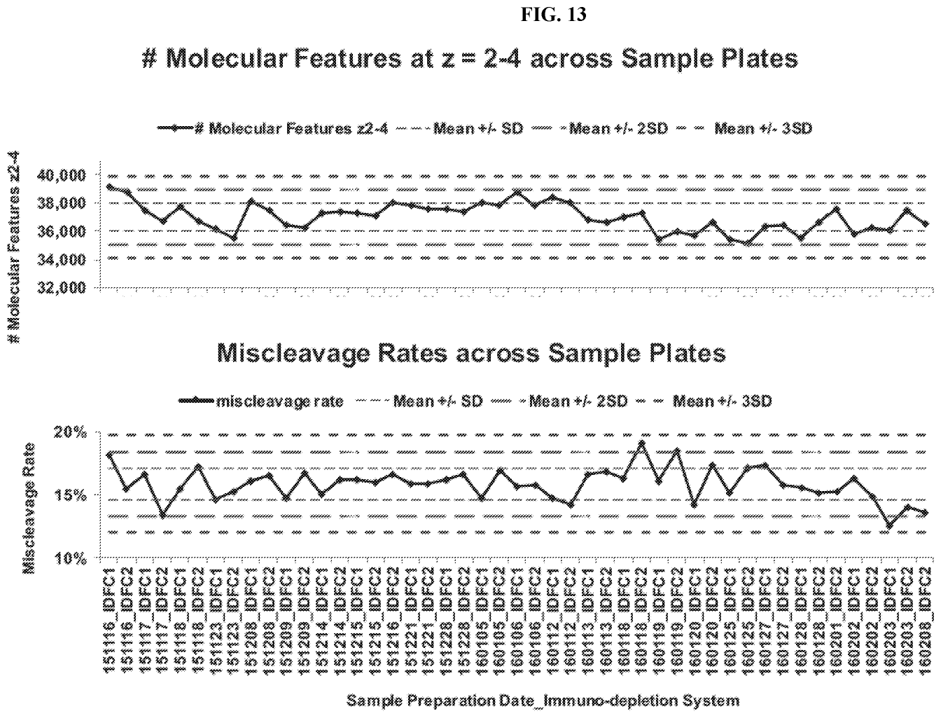

In addition, one out of four PQCs was processed in each sample batch (16 patient samples) for the purpose of monitoring immuno-depletion as well as trypsin digestion efficiency. Following sample processing and prior to the start of the biomarker study data collection, the single PQC from each sample batch was analyzed by two separate injections on a 6550 Q-TOF (Agilent technologies). A full scan MS1 analysis provided information on the abundance of molecular features (z=2-4), whereas the MS2 data dependent acquisition (DDA) analysis provided information on the identification of immuno-depleted Human 14 proteins and the missed cleavage rate as a measure of digestion efficiency. The molecular feature counts (z=2-4) and missed cleavage rate of the PQC on a total of 47 plates demonstrated reproducibility in both the immuno-depletion and trypsin digestion (FIG. 13). Both metrics for the PQC were within the +/−3 SD range throughout the study. The MS2 analysis of each PQC further supported high efficiency in immuno-depletion of the top-14 proteins. For 22 out of 47 PQCs, no top-14 proteins were detected. For the remaining 25 batches, one or two top-14 proteins were detected in PQCs while MS1 EIC peak AUC is −104 whereas AUCs of non-top-14 proteins are from 103 to 106.

### Monitoring LC-MS Performance

An essential requirement of a biomarker discovery study is establishing confidence in the proteomic data set. In the study presented here, data were acquired over a four-month period across two LC-MS systems, therefore monitoring the intra- and inter-day reproducibility within and between LC-MS systems was essential to safeguarding confidence in the results. PQCs, a SIS peptide mixture, and selected QC transitions were used to test system suitability prior to data collection, and to monitor the performance of each LC-MS system during sample batch analysis.

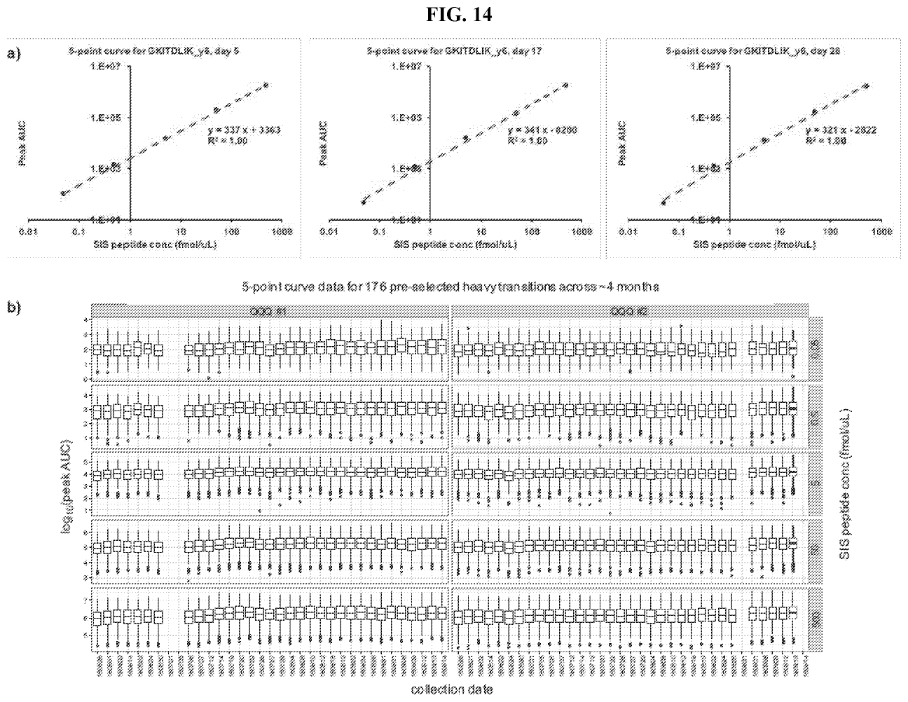

An SST was performed using a 5-point log-serial dilution of SIS peptide mixture in solvent at the start of each worklist. This provided real-time information on the state and performance level of each LC-MS system prior to initiating sample data collection. Each set of 5 injections of the SIS peptide mixture (0.05, 0.5, 5, 50, and 500 fmol/μL) was monitored for RT shift and signal intensity. Each day, 95% of the observed RTs were within 5 seconds of expected, passing quality criteria required to run samples. Heavy peak AUCs of 176 pre-selected QC transitions were consistent across 33 running days on two Agilent 6490 QQQs (FIG. 14). MS performance was also consistent across instruments, with heavy transition peak AUCs between two QQQs within one log unit of each other for each standard concentration level (FIG. 14). Dynamic ranges across five concentration levels were approximately four log units, with ten-fold increase of signal intensity between two adjacent concentration levels (FIG. 14).

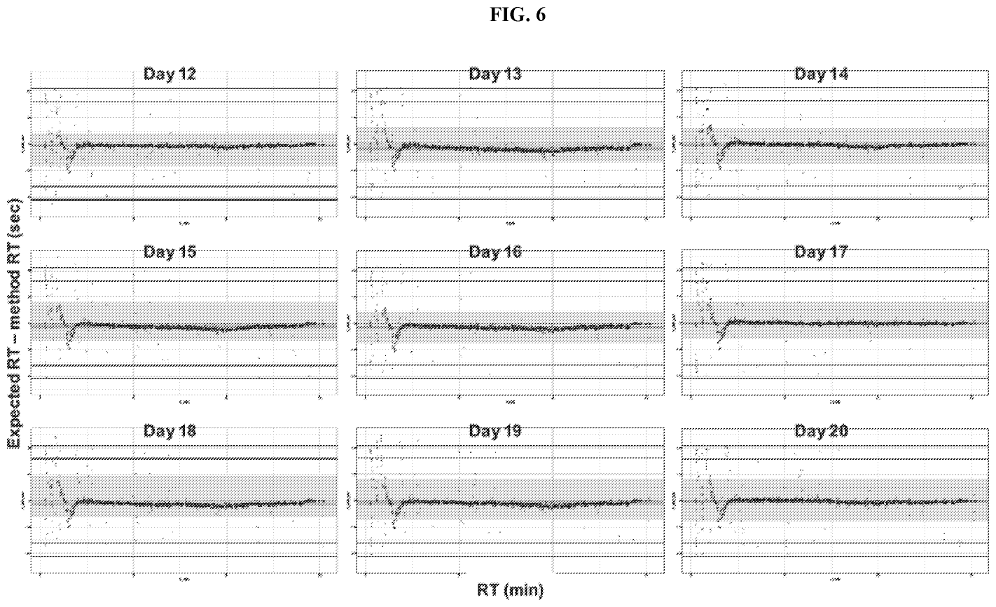

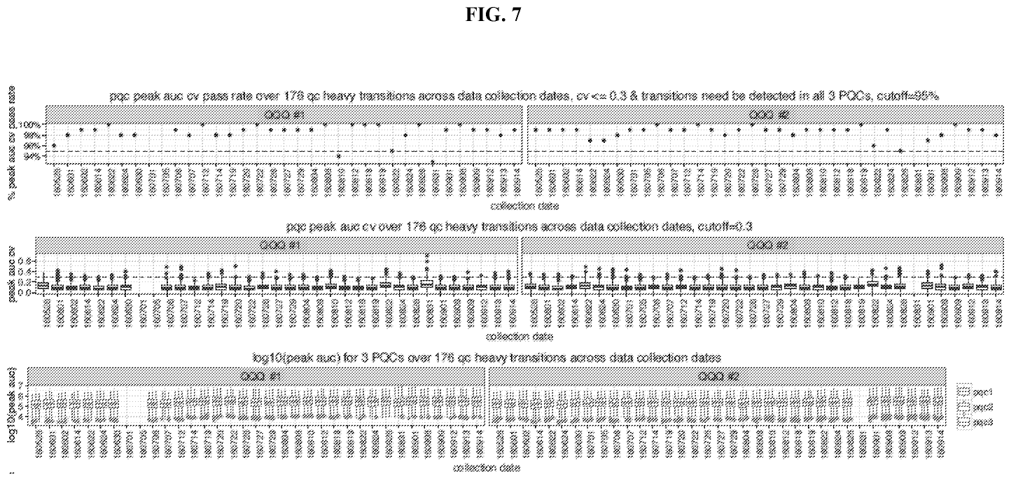

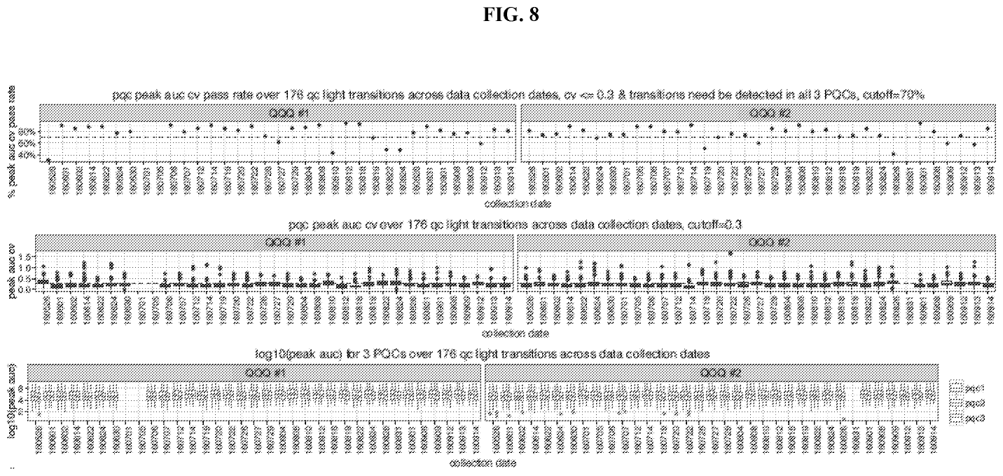

While confirming acceptable performance of the LC-MS system prior to data collection was essential, establishing confidence in the results acquired over a 21-hour sample batch run period was equally important. In this study, reference materials were three PQCs spiked with SIS peptide mixture, interleaved between study samples to run at the beginning, middle, and end of each day's runs. Each PQC was used to monitor both the LC and MS performance. To monitor LC performance, the peak apex elution of each heavy transition from the first PQC run each day was used to monitor RT shift; the acceptance criterion for each peak permitted a maximum 15-second shift in peak elution. FIG. 6 shows RT shifts for all the 1552 heavy transitions for nine consecutive running days on one Agilent QQQ. 95% of the 1552 heavy transitions had RT shift <10 seconds, thus passing quality criteria. To monitor MS performance, 176 QC transition pairs from PQCS were monitored. Each transition's heavy and light peak AUCs and their CVs were used. These can be visualized in control charts (FIGS. 7 & 8) that were automatically generated to monitor the peak AUCs for the 176 heavy and 176 light QC transitions in PQCs within a run and over days. The CVs across each single day's processing runs were evaluated and compared to 30% as the quality reference. Any observation above the 30% CV was considered outside of the acceptable range for intra-batch reproducibility. Overall, about 95% of the 176 heavy transitions and approximately 70% of the 176 light transitions had CV <=30% over the 67 batches across two LC-MS systems in a four-month data collection period. FIG. 7 and FIG. 8 show several clusters of heavy transitions including QQQ #1 on the left and QQQ #2 on the right. The top row indicates PQC peak AUC CV pass rate over 176 heavy transitions across data collection dates with a cv <=0.3 and requiring the transitions need to be detected in all 3 PQCs. The middle row indicates PQC peak AUC CV pass rate over 176 heavy transitions across data collection dates with a cv <=0.3. The bottom row indicates log 10 (peak AUC) for the 3 PQCs over 176 heavy transitions across data collection dates. The bottom row shows the PQC clusters with PQC1, PQC2, and PQC3 in order from left to right at each collection date.

In some embodiments, the consistency in heavy transition performance was achieved by adhering to a daily maintenance checklist for the HPLC, the QQQ, or both. High intra-batch CVs of 176 light transitions would trigger an investigation into either the instrument performance or sample processing. In actuality, no failures were observed in quality controls in the sample processing or system suitability testing. In addition, automated data processing permitted real time monitoring of trends in LC retention time and MS response. This allowed the operator to stop the instrument and remedy a problem if a component of the performance test failed to meet acceptance criteria.

### Data Processing: Evaluation of Univariate CRC Signal

Upon completion of data collection for the 1045 study samples, the data were compiled across all the samples for all 1552 transition pairs. Prior to study analysis, transitions were filtered according to three quality metrics. First, transitions were filtered according to their quantitative performance (see Methods “Assay analytical performance”). As described above, 1357 of the 1552 transitions were found to have quantitative performance. Second, both light and labeled peak pairs for each transition were filtered according to peak quality, assessed using a proprietary in-house machine learning tool (see Methods “Sample data processing”). Of the 1552 transitions, 1358 were found to have good quality for both light and labeled peaks throughout the study, 1290 of which also passed the first filter for quantitative performance. Finally, transitions were filtered to exclude those for which either light or labeled peaks were not evident in one or more of the study patient samples. Of the 1290 transitions that passed the first two filters, this step removed 338 transitions with missing values in one or more samples, leaving a total of 952 transitions passing all three quality filters. These 952 transitions covered 61.3% of the full 1552 transitions measured in the study. On the peptide level, these 952 transitions covered 529, or 82.5% of the 641 peptides in the study. On the protein level, these 952 transitions covered 345, or 88.0% of the 392 proteins in the study.

For each of these 952 transitions, endogenous concentration was calculated as the ratio of light/labeled peak area times the known spike-in concentration of the labeled peak. An overall assessment of univariate CRC signal in the dataset was performed. To this end, the CRC signal carried by each transition's endogenous concentrations in the 672-sample Discovery set was assessed. Each transition's univariate CRC signal was determined using ROC analysis to calculate a CRC vs non-CRC AUC, and its 95% confidence interval, in the 672-sample Discovery set.

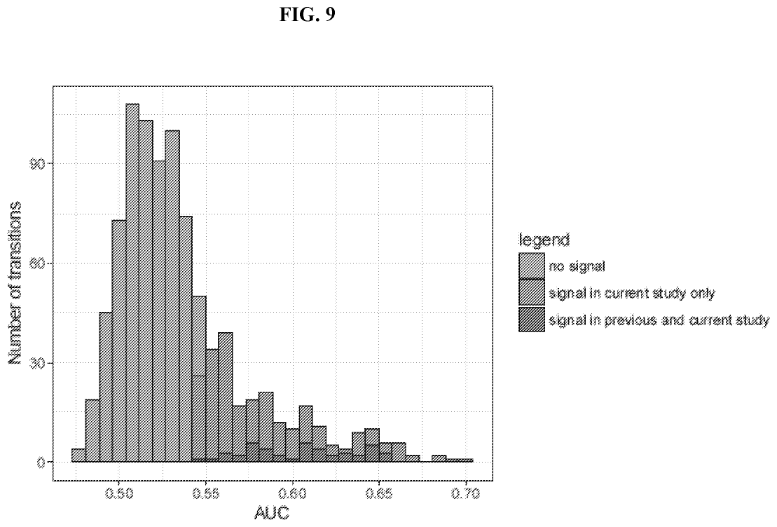

Of the 952 transitions considered in this analysis, 252 transitions, covering 127 unique proteins, were found to have AUCs with confidence intervals that excluded 0.50, indicating potential as single biomarkers (FIG. 9). Of these, 207 transitions were from 109 proteins that either did not produce signal or were not evaluated in our earlier targeted proteomics study. Since all the transitions had been selected based on previous studies (CPTAC or literature review), these 109 proteins can be considered as newly verified CRC biomarkers that are operable in the symptomatic population represented by our sample set. By contrast, the same AUC analysis applied to our earlier targeted proteomics study would have shown univariate CRC signal for 63 transitions covering 41 unique proteins. The increased number of transitions carrying univariate signal in the current study can be attributed to two factors. First, we used a Discovery sample set that was 4.9 times larger in the current study (672 samples in the current study, vs 138 samples in the earlier study), narrowing AUC confidence intervals and easing identification of valid signal. Second, we targeted about twice as many proteins in the current study (392 in the current study, vs 187 in the earlier study). FIG. 9 shows shaded bars corresponding to no signal beginning at below 0.50 AUC and ending at up to 0.55 AUC. The shaded bars corresponding to transitions identified in both the previous and current study only are shown in the bottom section of the shaded bars beginning at just below 0.55 AUC and ending at just past 0.65 AUC. The top section of the shaded bars (delineated by a horizontal line within each bar separating the top from the bottom sections) correspond to signal/transitions detected only in the current study. These transitions detected only in the current section begin at just below 0.55 AUC and extend up to about 0.70 AUC. Thus, a number of high AUC transitions were detected in the current study that were not present in the earlier study as shown by the section between about 0.65 AUC to about 0.70 AUC which have new transitions.

### Example 12—Colorectal Cancer Status: Protein Biomarker Panels

### Patient Samples

Plasma samples were taken from the Endoscopy II collection, described in Blume et al., 2016. The particular samples used in TPv2 were from the same 1,045 patients used to develop the SPCv1 CRC test, and are described in detail in Croner et al., unpublished. Briefly, the 1,045 samples were assigned to a 672-sample discovery set and a 373-sample validation set. The discovery set contained 373 samples in which the proportions of diagnostic groups were representative of the intent-to-test (ITT) population, and 299 additional CRC (176) and advanced adenoma (123) samples. The validation set contained 373 samples with ITT proportions of diagnostic groups. There was no overlap between the samples in the discovery and validation sets.

### Assays

The sample concentrations of targeted peptide ions were obtained using a dynamic MRM method on MS instruments. Target selection, assay development, and initial (pre-classifier) data processing are described in detail in You et al., 2018.

### Classifier Build and Validation Process

Supervised classifiers were built using API's “simple grid” approach applied to data from the 672-sample discovery set. For each simple grid process, all possible classifiers defined by a set of parameters were built using ten iterations of 10-fold cross validation applied to the discovery set; the classifier with the highest median merged AUC across the ten iterations was then selected as the top build for that grid. In total, 58 simple grids were run. All the grids used glmnet feature selection within each fold. However, the grids varied in the range of feature counts considered, whether age and/or gender were included as predictor candidates, the subset of transitions included as predictor candidates, whether transition concentration data were log 2-transformed, whether ratios based on transitions and other features were included as predictor candidates, whether data scaling was tested, the classifier algorithms used, the supervised discrimination performed (CRC vs non-CRC, or CRC vs “No comorbidity-no finding” diagnostic group [NCNF, cleanest controls]), and/or the portion of the discovery set used (full discovery set or ITT subset). Further details about the simple grid approach can be found in Croner et al., 2017 and Croner et al., unpublished.

Final models from the most promising grid builds were used in Indeterminate or “NoCall” (NoC) analyses. NoC analyses were applied to the CRC vs non-CRC discrimination within the ITT subset of the discovery set. NoC analyses aimed to determine a contiguous range of model scores such that samples receiving scores in that range would not receive a final model-based CRC call, thus enhancing the overall performance of the model. Further details about NoC analyses can be found in Croner et al., 2017 and Croner et al., unpublished.

Six of the best-performing classifiers and their associated NoC regions were then tested in the separate validation set. Validation was considered a success if 1) the validation AUC was either not statistically distinguishable from the discovery AUC or was statistically distinguishable from and higher than the discovery AUC, and 2) the validation AUC was statistically distinguishable from and greater than the univariate age AUC in the validation set. For successful validations, the validation AUC was also compared with the SPCv1 validation AUC; in this comparison, the study goal of at least equivalent performance to SPCv1 would be met by finding that either the two AUCs were not statistically distinguishable, or that they were statistically distinguishable with the TPv2 AUC having the higher value.

### Five Groups of Simple Grids

Despite the wide variation across simple grid configurations, the 58 grid builds can be grouped into five general approaches, described below. The five approaches differ in the pool of features from which the simple grid's glmnet feature selection pulled candidate predictors for each fold of each build.

### Standard Builds

These builds used simplistic and pre-planned feature sets as pools of candidate predictors. These pools included the sets of transitions and demographics in each of the two main data matrices provided by Atet Kao (AK) (see below). They also included the set of 252 transitions with significant CRC vs non-CRC signal, as described in You et al., 2018.

### Specialized Features: Ratios

These builds included ratios—ratios of transition concentrations, and ratios involving both patient age and transition concentrations—in the pool of candidate predictors. For these builds, all possible ratios were calculated for limited feature sets. Specifically, they were calculated for the 252 transitions with CRC vs non-CRC signal, and for the transitions involved in the best AK 2016 classifier (see below).

### Specialized Feature Subsets: A Few Strong Predictors

These builds aimed to use a small number of predictors, and pulled predictor candidates only from a list of 23 single features and feature ratios shown to have CRC vs NCNF univariate AUCs >=0.85 in the discovery set. These 23 features and ratios were as follows:

| # | Biomarker_peptidefragment |
|---|---|
| 1 | A2GL_DLLLPQPDLR_b3 |
| 2 | A2GL_VAAGAFQGLR_y7 |
| 3 | A2GL_VAAGAFQGLR_y8 |
| 4 | ALS_ELDLSR_y3 |
| 5 | ALS_LFQGLGK_y4 |
| 6 | ALS_LFQGLGK_y6 |
| 7 | IBP3_FLNVLSPR_y3 |
| 8 | IBP3_YGQPLPGYTTK_y6 |
| 9 | patient_age |
| 10 | PTPRJ_VALTGVR_y5 |
| 11 | THRB_IYIHPR_y4 |
| 12 | A2GL_VAAGAFQGLR_y7/ALS_LFQGLGK_y6 |
| 13 | A2GL_VAAGAFQGLR_y8/ALS_LFQGLGK_y6″ |
| 14 | A2GL_VAAGAFQGLR_y7/ALS_LFQGLGK_y4 |
| 15 | PTPRJ_VALTGVR_y5/patient_age |
| 16 | A2GL_VAAGAFQGLR_y7/PTPRJ_VALTGVR_y5 |
| 17 | A2GL_VAAGAFQGLR_y8/ALS_LFQGLGK_y4 |
| 18 | A2GL_VAAGAFQGLR_y7/IBP3_FLNVLSPR_y3 |
| 19 | A2GL_VAAGAFQGLR_y7/THRB_IYIHPR_y4 |
| 20 | A2GL_VAAGAFQGLR_y7/IBP3_YGQPLPGYTTK_y6 |
| 21 | ALS_LFQGLGK_y4/patient_age |
| 22 | A2GL_DLLLPQPDLR_b3/ALS_LFQGLGK_y6 |
| 23 | A2GL_VAAGAFQGLR_y7/ALS_ELDLSR_y3 |

### Specialized Feature Subsets: Additional Feature Selection

These builds pulled predictor candidates from one of three specialized feature subsets determined by ten feature selection algorithms that differed from the glmnet approach used in simple grids.

Both TPv1 (Jones et al., 2016), and AK 2016 builds (see below) used a variety of feature selection methods encompassed in the R package known as FSelector. To increase the power of the simple grids, ten FSelector feature selection algorithms were applied to three promising subsets of features; then simple grid builds pulled candidate predictors only from features selected by these additional algorithms.

The ten FSelector algorithms applied were correlation, consistency, linear correlation, rank correlation, information gain, gain ratio, symmetrical uncertainty, oneR, random forest, and relief. The three promising transition subsets to which these algorithms were applied were the 252 transitions with univariate CRC signal (see You et al., 2018), the 23 transitions and ratios with univariate CRC AUCs (CRC vs NCNF) >=0.85, and the 974 transitions with complete measures and passing peak quality metrics (from the second data matrix described below). For each feature subset, the features selected by the ten algorithms were pooled and then used as a single list of features from which the simple grid builds would pull candidate predictors in a separate set of builds.

### Specialized Feature Subsets: AK 2016 Classifiers

These builds pulled predictors from a specialized subset of 23 transitions based on AK 2016 classifier builds.

AK built TPv2 classifiers using the “expanded grid” process in late 2016. The expanded grid differed from the simple grid primarily in using a wider range of feature selection methods. In the past, some of API's best-performing classifiers resulted from AK's expanded grid. Thus, one strategy for the new TPv2 classifiers described here was to limit features in some of the new builds to those used in the best AK build. To that end, AK's 2016 classifier files were compiled and explored to identify these features.

The best 2016 TPv2 build was an 11-feature glmboost, with median merged test AUC of 0.92 from discovery cross-validation. This build was for a CRC vs NCNF discrimination. For this particular model, 32 features (31 transitions and age) were selected as predictors in various versions of the 11-feature glmboost model. Ideally, all of these features would be explored with new classifiers using the final classifier matrices provided by AK to the team. However, only 23 of the 31 transitions appeared in the preferred data matrix (the matrix with complete measures from transitions that passed peak quality checks, see below). In addition, for those transitions that were represented in both AK builds' and the 2018 builds' data matrices, the concentration values differed numerically between the two files; this was likely due to the use of different algorithms for calculating raw peak area—probably pipeline-based raw peaks for the best AK build, and AKRawV1 raw peaks for the files distributed to the classifier team. Despite these issues, a reasonable approach was to use the 23 features appearing in both the AK and classifier team matrices, when performing the subset of the new builds aimed at exploring the best AK build. These 23 features were as follows:

| # | Biomarker_peptidefragment |
|---|---|
| 1 | A2GL_VAAGAFQGLR_y7 |
| 2 | A2GL_VAAGAFQGLR_y8 |
| 3 | ACTBM_SYELPDGQVITIGNER_y12 |
| 4 | ALS_LFQGLGK_y4 |
| 5 | ALS_LFQGLGK_y6 |
| 6 | APOC4_AWFLESK_y3 |
| 7 | APOE_AQAWGER_y5 |
| 8 | APOL1_ALDNLAR_y4 |
| 9 | GUC2A_EPNAQEILQR_y3 |
| 10 | I10R1_EYEIAIR_y3 |
| 11 | ITIH2_TAGLVR_y3 |
| 12 | KAIN_LELHLPK_y6 |
| 13 | LYNX1_VLSNTEDLPLVTK_y8 |
| 14 | PON1_SLLHLK_b4 |
| 15 | PON1_SLLHLK_b5 |
| 16 | PREX2_AFYLDK_y5 |
| 17 | PTPRJ_VALTGVR_y5 |
| 18 | RET4_YWGVASFLQK_b4 |
| 19 | SPP24_DALSASVVK_y6 |
| 20 | TFR1_LYWDDLK_y5 |
| 21 | TFR1_SGVGTALLLK_b3 |
| 22 | TFR1_SGVGTALLLK_y7 |
| 23 | TNF15_AHLTVVR_y4 |

### Peak Images

To enable manual review of peak quality, peak images were built for transitions that appeared in top classifiers. The process for building these images was based on that employed by AK in 2016, when an effort was made to produce image files for all of the TPv2 transitions. This 2016 effort was halted before completion, in part because of the long time required to build the images. Here, the same process was used to build image files for just the subset of transitions playing important roles in the 2018 classifiers.

### Classifier Input Files

A peak identification algorithm was used for calculating raw peak areas. An alternative would have been to use the API pipeline algorithm. (Note: The pipeline algorithm was likely used to calculate peak areas for data used in AK's original classifier builds.)

Some data files contain only those transitions that had valid measures in all 1,045 samples. Valid measures were those with non-NA raw peak areas for SIS peaks.

Some data files considered only transitions with endogenous and SIS peaks assigned to peak quality group 1 or 2 when building the data file. Thus the data file contains only those transitions that were assessed as good quality and that had valid measures in all 1,045 samples. The peak quality tool used was a random forest classifier that assigns peaks to one of three quality groups, with group 3 being the lowest quality group.

Comparison of Measures from Three Endoscopy II Studies

Additional work was performed comparing the various measures API generated for the Endoscopy II samples. These included CRC05 ELISA, CRC06 MSD, CRC05 MRM (TPv2) measures.

### Results

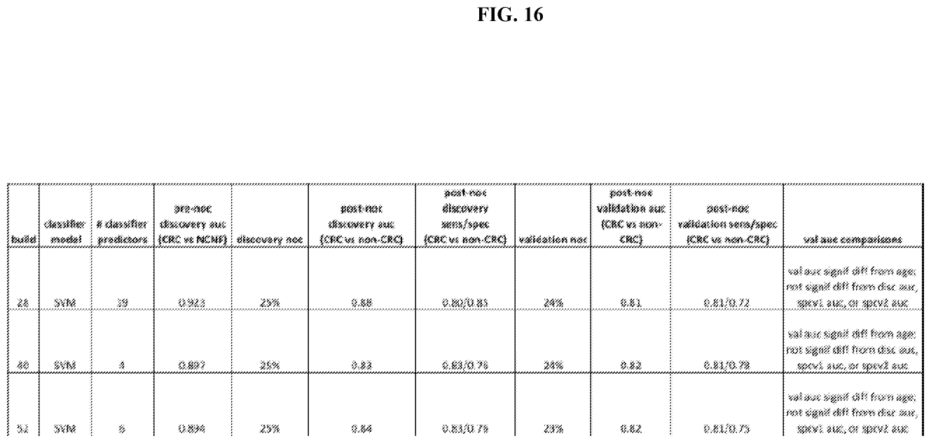

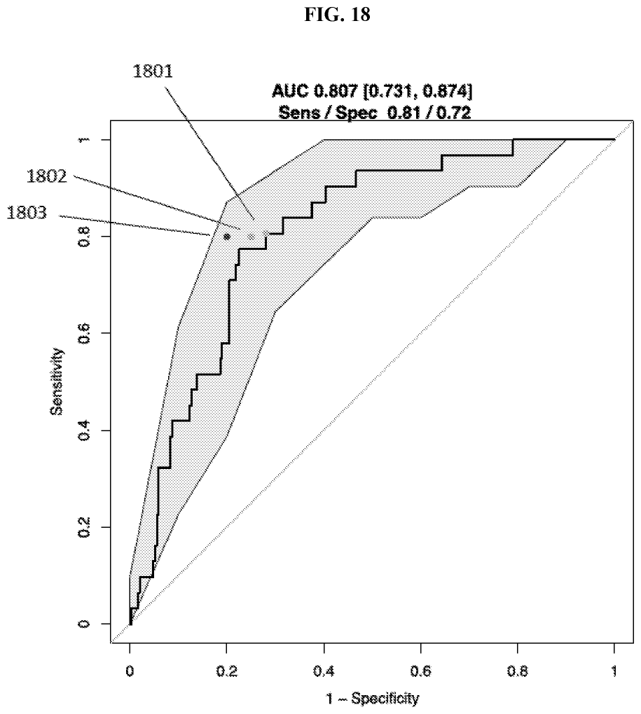

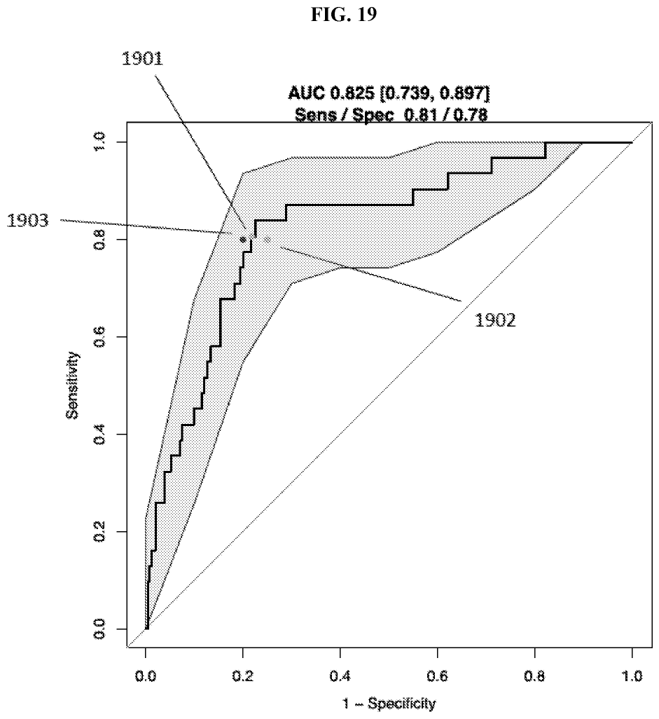

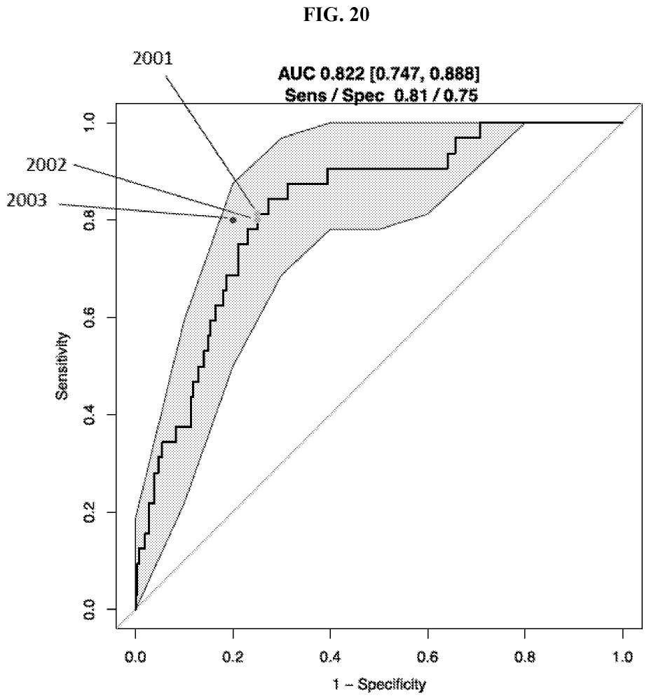

Of the 58 simple grids performed, 17 gave rise to classifiers that were subjected to NoC analyses. Validation was attempted for six of these 17 classifiers, and succeeded for three. These three successful validations came from grid build numbers 28, 40, and 52. Further details about the 58 grids performed are presented in the Discussion. Here we offer FIG. 16 summarizing the characteristics and findings for the validated classifiers, Table 7 listing the predictors used in these classifiers, and FIGS. 18-20 showing the validation ROCs. The best-performing classifier was that from build 40. This was a 4-predictor SVM; the predictors include two ratios (both have age in their denominator), one single transition, and age alone. With 23% NoC in validation, this classifier had CRC vs non-CRC sens/spec of 0.81/0.78, matching that of the SPCv1 CRC test.

TABLE 7
Predictors in each of the three
validated classifiers. Two predictors
for model 40 are ratios.

| | model | model | model |
|---|---|---|---|
| predictor | 28 | 40 | 52 |
| A2GL_VAAGAFQGLR_y7 | x | x | x |
| A2GL_VAAGAFQGLR_y8 | x | | |
| ACTBM_SYELPDGQVITIGNER_y12 | x | | |
| ALS_LFQGLGK_y4 | x | | |
| ALS_LFQGLGK_y4/patient_age | | x | |
| ALS_LFQGLGK_y6 | x | | x |
| APOC4_AWFLESK_y3 | x | | |
| APOE_AQAWGER_y5 | x | | |
| APOL1_ALDNLAR_y4 | x | | |
| CHLE_EFQEGLK_y3 | x | | |
| GELS_AGALNSNDAFVLK_b4 | | | x |
| I10R1_EYEIAIR_y3 | x | | |
| ITIH2_TAGLVR_y3 | x | | |
| KAIN_LELHLPK_y6 | x | | |
| patient_age | x | x | x |
| PON1_SLLHLK_b5 | x | | |
| PTPRJ_VALTGVR_y5 | x | | x |
| PTPRJ_VALTGVR_y5/patient_age | | x | |
| SPP24_DALSASVVK_y6 | x | | |
| TFR1_SGVGTALLLK_b3 | x | | |
| TFR1_SGVGTALLLK_y7 | x | | x |
| TNF15_AHLTVVR_y4 | x | | |
| Total classifier predictors | 19 | 4 | 6 |
| Total unique transitions | 18 | 3 | 5 |

What is claimed is:

1. A method of assessing a colorectal health risk status in an individual, comprising steps of:
a) obtaining a circulating blood sample from said individual; and
b) obtaining a biomarker panel level for at least two of A2GL, ALS, and PTPRJ of said circulating blood sample, and assessing colorectal health risk status.

2. The method of claim 1, wherein said biomarker panel further comprises an individual age.

3. The method of claim 1, wherein said colorectal cancer status comprises at least one of early CRC and advanced CRC.

4. The method of claim 1, wherein said colorectal cancer status comprises at least one of advanced adenoma, Stage 0 CRC, stage I CRC, Stage II CRC, stage III CRC, and stage IV CRC.

5. The method of claim 1, wherein said biomarker panel comprises no more than 20 proteins.

6. The method of claim 1, wherein said biomarker panel comprises no more than 10 proteins.

7. The method of claim 1, wherein said categorizing has a sensitivity of at least 70% and a specificity of at least 70%.

8. The method of claim 1, further comprising performing a treatment regimen in response to said categorizing.

9. The method of claim 8, wherein said treatment regimen comprises at least one of chemotherapy, radiation, immunotherapy, administration of a biologic therapeutic agent, polypectomy, partial colectomy, low anterior resection or abdominoperineal resection and colostomy.

10. The method of claim 1, further comprising transmitting a report of results of said categorizing to a health practitioner.

11. The method of claim 10, wherein said report indicates a sensitivity of at least 70%.

12. The method of claim 10, wherein said report indicates a specificity of at least 70%. 14.

13. The method of claim 10, wherein said report indicates a recommendation for a treatment regimen comprising at least one of chemotherapy, radiation, immunotherapy, administration of a biologic therapeutic agent, polypectomy, partial colectomy, low anterior resection or abdominoperineal resection and colostomy.

14. The method of claim 10, wherein said report indicates a recommendation for a colonoscopy.

15. The method of claim 10, wherein said report indicates a recommendation for undergoing an independent cancer assay.

16. The method of claim 10, wherein said report indicates a recommendation for undergoing a stool cancer assay.

17. The method of claim 1, further comprising performing a stool cancer assay in response to said categorizing.

18. The method of claim 1, further comprising continued monitoring for a period of 3 months or greater.

19. The method of claim 1, further comprising continued monitoring for a period of between 3 months and 24 months.

20. The method of claim 1, wherein said obtaining said protein levels comprises subjecting said biological sample to a mass spectrometric analysis.

21. The method of claim 20, wherein said mass spectrometric analysis is evaluated according to at least one process control step.

22. The method of claim 21, wherein the process control step comprises using at least one system suitability test (SST) run to assess liquid chromatography (LC) and mass spectrometry (MS) performance prior to the mass spectrometric processing.

23. The method of claim 1, wherein said obtaining said protein levels comprises subjecting said biological sample to an affinity assay.

24. The method of claim 21, wherein said affinity assay comprises an immunoassay analysis of said biological sample.

25. The method of claim 21, wherein said affinity assay comprises an aptamer analysis of said biological sample.

26. The method of claim 21, wherein said affinity assay comprises assessing said biological sample according to a quality control (QC) parameter.

27. The method of claim 26, wherein the QC parameter comprises at least one of sample integrity, sample elution efficiency, sample storage condition, and internal standard monitoring.

28. A method of generating a biomarker panel for assessing a health status, comprising:
a) identifying candidate biomarkers having an association with the health status; and
b) performing mass spectrometric processing on at least a fragment of a plurality of candidate biomarker proteins derived from the candidate biomarkers to determine biomarkers suitable for assessing a health status;
wherein the processing comprises at least one process control step.

29. The method of claim 28, wherein the at least one process control step comprises using at least one system suitability test (SST) run to assess liquid chromatography (LC) and mass spectrometry (MS) performance prior to the mass spectrometric processing.

30. The method of claim 29, wherein the SST comprises determining LC-MS performance by running a SIS standard curve in log-serial dilution.

31. The method of claim 30, further comprising performing a quality control check requiring at least about a 10-fold difference in MS signal between any two adjacent concentration levels, and a dynamic range of approximately four log units across the standard curve.

32. The method of claim 28, wherein the SST comprises determining LC performance by monitoring heavy transitions of internal standards for RT stability.

33. The method of claim 32, wherein monitoring heavy transitions comprises tracking RT shift between a detected value and a scheduled RT.

34. The method of claim 31, further comprising performing a quality control check requiring the upper 95% confidence interval of RTs of heavy transitions are no more than 10% from the margin from the margins of LC-MS acquisition windows.

35. The method of claim 28, wherein the at least one process control step comprises monitoring flow-through AUC during immunodepletion, monitoring of TPA results for sample processing and immunodepletion efficiency, sample preparation customization depending on the TPA result of each individual sample, or any combination thereof.

36. The method of claim 28, wherein the at least a fragment comprises a proteotypic peptide.

37. The method of claim 28, wherein the at least a fragment comprises a full length protein.


SEQUENCE LISTING (71 sequences)

SEQ ID NO: 1 - protein, 347 aa, Homo sapiens
MSSWSRQRPKSPGGIQPHVSRTLFLLLLLAASAWGVTLSPKDCQVFRSDHGSSISCQPPAEIPGYLPADTVHLAVEFFNLTHLPANLLQGASKLQELHLSSNGLESLSPEFLRPVPQLRVLDLTRNALTGLPPGLFQASATLDTLVLKENQLEVLEVSWLHGLKALGHLDLSGNRLRKLPPGLLANFTLLRTLDLGENQLETLPPDLLRGPLQLERLHLEGNKLQVLGKDLLLPQPDLRYLFLNGNKLARVAAGAFQGLRQLDMLDLSNNSLASVPEGLWASLGQPNWDMRDGFDISGNPWICDQNLSDLYRWLQAQKDKMFSQNDTRCAGPEAVKGQTLLAVAKSQ

SEQ ID NO: 2 - protein, 375 aa, Homo sapiens
MDDDTAVLVIDNGSGMCKAGFAGDDAPQAVFPSIVGRPRHQGMMEGMHQKESYVGKEAQSKRGMLTLKYPMEHGIITNWDDMEKIWHHTFYNELRVAPEEHPILLTEAPLNPKANREKMTQIMFETFNTPAMYVAIQAVLSLYTSGRTTGIVMDSGDGFTHTVPIYEGNALPHATLRLDLAGRELTDYLMKILTERGYRFTTTAEQEIVRDIKEKLCYVALDSEQEMAMAASSSSVEKSYELPDGQVITIGNERFRCPEALFQPCFLGMESCGIHKTTFNSIVKSDVDIRKDLYTNTVLSGGTTMYPGIAHRMQKEITALAPSIMKIKIIAPPKRKYSVWVGGSILASLSTFQQMWISKQEYDESGPSIVHRKCF

SEQ ID NO: 3 - protein, 605 aa, Homo sapiens
MALRKGGLALALLLLSWVALGPRSLEGADPGTPGEAEGPACPAACVCSYDDDADELSVFCSSRNLTRLPDGVPGGTQALWLDGNNLSSVPPAAFQNLSSLGFLNLQGGQLGSLEPQALLGLENLCHLHLERNQLRSLALGTFAHTPALASLGLSNNRLSRLEDGLFEGLGSLWDLNLGWNSLAVLPDAAFRGLGSLRELVLAGNRLAYLQPALFSGLAELRELDLSRNALRAIKANVFVQLPRLQKLYLDRNLIAAVAPGAFLGLKALRWLDLSHNRVAGLLEDTFPGLLGLRVLRLSHNAIASLRPRTFKDLHFLEELQLGHNRIRQLAERSFEGLGQLEVLTLDHNQLQEVKAGAFLGLTNVAVMNLSGNCLRNLPEQVFRGLGKLHSLHLEGSCLGRIRPHTFTGLSGLRRLFLKDNGLVGIEEQSLWGLAELLELDLTSNQLTHLPHRLFQGLGKLEYLLLSRNRLAELPADALGPLQRAFWLDVSHNRLEALPNSLLAPLGRLRYLSLRNNSLRTFTPQPPGLERLWLEGNPWDCGCPLKALRDFALQNPSAVPRFVQAICEGDDCQPPAYTYNNITCASPPEVVGLDLRDLSEAHFAPC

SEQ ID NO: 4 - protein, 127 aa, Homo sapiens
MSLLRNRLQALPALCLCVLVLACIGACQPEAQEGTLSPPPKLKMSRWSLVRGRMKELLETVVNRTRDGWQWFWSPSTFRGFMQTYYDDHLRDLGPLTKAWFLESKDSLLKKTHSLCPRLVCGDKDQG

SEQ ID NO: 5 - protein, 317 aa, Homo sapiens [sequence omitted]

SEQ ID NO: 6 - protein, 398 aa, Homo sapiens
MEGAALLRVSVLCIWMSALFLGVGVRAEEAGARVQQNVPSGTDTGDPQSKPLGDWAAGTMDPESSIFIEDAIKYFKEKVSTQNLLLLLTDNEAWNGFVAAAELPRNEADELRKALDNLARQMIMKDKNWHDKGQQYRNWFLKEFPRLKSELEDNIRRLRALADGVQKVHKGTTIANVVSGSLSISSGILTLVGMGLAPFTEGGSLVLLEPGMELGITAALTGITSSTMDYGKKWWTQAQAHDLVIKSLDKLKEVREFLGENISNFLSLAGNTYQLTRGIGKDIRALRRARANLQSVPHASASRPRVTEPISAESGEQVERVNEPSILEMSRGVKLTDVAPVSFFLVLDVVYLVYESKHLHEGAKSETAEELKKVAQELEEKLNILNNNYKILQADQEL

SEQ ID NO: 7 - protein, 602 aa, Homo sapiens
MHSKVTIICIRFLFWFLLLCMLIGKSHTEDDIIIATKNGKVRGMNLTVFGGTVTAFLGIPYAQPPLGRLRFKKPQSLTKWSDIWNATKYANSCCQNIDQSFPGFHGSEMWNPNTDLSEDCLYLNVWIPAPKPKNATVLIWIYGGGFQTGTSSLHVYDGKFLARVERVIVVSMNYRVGALGFLALPGNPEAPGNMGLFDQQLALQWVQKNIAAFGGNPKSVTLFGESAGAASVSLHLLSPGSHSLFTRAILQSGSFNAPWAVTSLYEARNRTLNLAKLTGCSRENETEIIKCLRNKDPQEILLNEAFVVPYGTPLSVNFGPTVDGDFLTDMPDILLELGQFKKTQILVGVNKDEGTAFLVYGAPGFSKDNNSIITRKEFQEGLKIFFPGVSEFGKESILFHYTDWVDDQRPENYREALGDVVGDYNFICPALEFTKKFSEWGNNAFFYYFEHRSSKLPWPEWMGVMHGYEIEFVFGLPLERRDNYTKAEEILSRSIVKRWANFAKYGNPNETQNNSTSWPVFKSTEQKYLTLNTESTRIMTKLRAQQCRFWTSFFPKVLEMTGNIDEAEWEWKAGFHRWNNYMMDWKNQFNDYTSKKESCVGL

SEQ ID NO: 8 - protein, 782 aa, Homo sapiens
MAPHRPAPALLCALSLALCALSLPVRAATASRGASQAGAPQGRVPEARPNSMVVEHPEFLKAGKEPGLQIWRVEKFDLVPVPTNLYGDFFTGDAYVILKTVQLRNGNLQYDLHYWLGNECSQDESGAAAIFTVQLDDYLNGRAVQHREVQGFESATFLGYFKSGLKYKKGGVASGFKHVVPNEVVVQRLFQVKGRRVVRATEVPVSWESFNNGDCFILDLGNNIHQWCGSNSNRYERLKATQVSKGIRDNERSGRARVHVSEEGTEPEAMLQVLGPKPALPAGTEDTAKEDAANRKLAKLYKVSNGAGTMSVSLVADENPFAQGALKSEDCFILDHGKDGKIFVWKGKQANTEERKAALKTASDFITKMDYPKQTQVSVLPEGGETPLFKQFFKNWRDPDQTDGLGLSYLSSHIANVERVPFDAATLHTSTAMAAQHGMDDDGTGQKQIWRIEGSNKVPVDPATYGQFYGGDSYIILYNYRHGGRQGQIIYNWQGAQSTQDEVAASAILTAQLDEELGGTPVQSRVVQGKEPAHLMSLFGGKPMIIYKGGTSREGGQTAPASTRLFQVRANSAGATRAVEVLPKAGALNSNDAFVLKTPSAAYLWVGTGASEAEKTGAQELLRVLRAQPVQVAEGSEPDGFWEALGGKAAYRTSPRLKDKKMDAHPPRLFACSNKIGRFVIEEVPGELMQEDLATDDVMLLDTWDQVFVWVGKDSQEEEKTEALTSAKRYIETDPANRDRRTPITVVKQGFEPPSFVGWFLGWDDDYWSVDPLDRAMAELAA

SEQ ID NO: 9 - protein, 578 aa, Homo sapiens
MLPCLVVLLAALLSLRLGSDAHGTELPSPPSVWFEAEFFHHILHWTPIPNQSESTCYEVALLRYGIESWNSISNCSQTLSYDLTAVTLDLYHSNGYRARVRAVDGSRHSNWTVTNTRFSVDEVTLTVGSVNLEIHNGFILGKIQLPRPKMAPANDTYESIFSHFREYEIAIRKVPGNFTFTHKKVKHENFSLLTSGEVGEFCVQVKPSVASRSNKGMWSKEECISLTRQYFTVTNVIIFFAFVLLLSGALAYCLALQLYVRRRKKLPSVLLFKKPSPFIFISQRPSPETQDTIHPLDEEAFLKVSPELKNLDLHGSTDSGFGSTKPSLQTEEPQFLLPDPHPQADRTLGNREPPVLGDSCSSGSSNSTDSGICLQEPSLSPSTGPTWEQQVGSNSRGQDDSGIDLVQNSEGRAGDTQGGSALGHHSPPEPEVPGEEDPAAVAFQGYLRQTRCAEEKATKTGCLEEESPLTDGLGPKFGRCLVDEAGLHPPALAKGYLKQDPLEMTLASSGAPTGQWNQPTEEWSLLALSSCSDLGISDWSFAHDLAPLGCVAAPGGLLGSFNSDLVTLPLISSLQSSE

SEQ ID NO: 10 - protein, 946 aa, Homo sapiens
MKRLTCFFICFFLSEVSGFEIPINGLSEFVDYEDLVELAPGKFQLVAENRRYQRSLPGESEEMMEEVDQVTLYSYKVQSTITSRMATTMIQSKVVNNSPQPQNVVFDVQIPKGAFISNFSMTVDGKTFRSSIKEKTVGRALYAQARAKGKTAGLVRSSALDMENFRTEVNVLPGAKVQFELHYQEVKWRKLGSYEHRIYLQPGRLAKHLEVDVWVIEPQGLRFLHVPDTFEGHFDGVPVISKGQQKAHVSFKPTVAQQRICPNCRETAVDGELVVLYDVKREEKAGELEVFNGYFVHFFAPDNLDPIPKNILFVIDVSGSMWGVKMKQTVEAMKTILDDLRAEDHFSVIDFNQNIRTWRNDLISATKTQVADAKRYIEKIQPSGGTNINEALLRAIFILNEANNLGLLDPNSVSLIILVSDGDPTVGELKLSKIQKNVKENIQDNISLFSLGMGFDVDYDFLKRLSNENHGIAQRIYGNQDTSSQLKKFYNQVSTPLLRNVQFNYPHTSVTDVTQNNFHNYFGGSEIVVAGKFDPAKLDQIESVITATSANTQLVLETLAQMDDLQDFLSKDKHADPDFTRKLWAYLTINQLLAERSLAPTAAAKRRITRSILQMSLDHHIVTPLTSLVIENEAGDERMLADAPPQDPSCCSGALYYGSKVVPDSTPSWANPSPTPVISMLAQGSQVLESTPPPHVMRVENDPHFIIYLPKSQKNICFNIDSEPGKILNLVSDPESGIVVNGQLVGAKKPNNGKLSTYFGKLGFYFQSEDIKIEISTETITLSHGSSTFSLSWSDTAQVTNQRVQISVKKEKVVTITLDKEMSFSVLLHRVWKKHPVNVDFLGIYIPPTNKFSPKAHGLIGQFMQEPKIHIFNERPGKDPEKPEASMEVKGQKLIITRGLQKDYRTDLVFGTDVTCWFVHNSGKGFIDGHYKDYFVPQLYSFLKRP

SEQ ID NO: 11 - protein, 427 aa, Homo sapiens
MHLIDYLLLLLVGLLALSHGQLHVEHDGESCSNSSHQQILETGEGSPSLKIAPANADFAFRFYYLIASETPGKNIFFSPLSISAAYAMLSLGACSHSRSQILEGLGFNLTELSESDVHRGFQHLLHTLNLPGHGLETRVGSALFLSHNLKFLAKFLNDTMAVYEAKLFHTNFYDTVGTIQLINDHVKKETRGKIVDLVSELKKDVLMVLVNYIYFKALWEKPFISSRTTPKDFYVDENTTVRVPMMLQDQEHHWYLHDRYLPCSVLRMDYKGDATVFFILPNQGKMREIEEVLTPEMLMRWNNLLRKRNFYKKLELHLPKFSISGSYVLDQILPRLGFTDLFSKWADLSGITKQQKLEASKSFHKATLDVDEAGTEAAAATSFAIKFFSAQTNRHILRFNRPFLVVIFSTSTQSVLFLGKVVDPTKP

SEQ ID NO: 12 - protein, 355 aa, Homo sapiens [sequence omitted]

SEQ ID NO: 13 - protein, 1337 aa, Homo sapiens
MKPAAREARLPPRSPGLRWALPLLLLLLRLGQILCAGGTPSPIPDPSVATVATGENGITQISSTAESFHKQNGTGTPQVETNTSEDGESSGANDSLRTPEQGSNGTDGASQKTPSSTGPSPVFDIKAVSISPTNVILTWKSNDTAASEYKYVVKHKMENEKTITVVHQPWCNITGLRPATSYVFSITPGIGNETWGDPRVIKVITEPIPVSDLRVALTGVRKAALSWSNGNGTASCRVLLESIGSHEELTQDSRLQVNISGLKPGVQYNINPYLLQSNKTKGDPLGTEGGLDASNTERSRAGSPTAPVHDESLVGPVDPSSGQQSRDTEVLLVGLEPGTRYNATVYSQAANGTEGQPQAIEFRTNAIQVFDVTAVNISATSLTLIWKVSDNESSSNYTYKIHVAGETDSSNLNVSEPRAVIPGLRSSTFYNITVCPVLGDIEGTPGFLQVHTPPVPVSDFRVTVVSTTEIGLAWSSHDAESFQMHITQEGAGNSRVEITTNQSIIIGGLFPGTKYCFEIVPKGPNGTEGASRTVCNRTVPSAVFDIHVVYVTTTEMWLDWKSPDGASEYVYHLVIESKHGSNHTSTYDKAITLQGLIPGTLYNITISPEVDHVWGDPNSTAQYTRPSNVSNIDVSTNTTAATLSWQNFDDASPTYSYCLLIEKAGNSSNATQVVTDIGITDATVTELIPGSSYTVEIFAQVGDGIKSLEPGRKSFCTDPASMASFDCEVVPKEPALVLKWTCPPGANAGFELEVSSGAWNNATHLESCSSENGTEYRTEVTYLNFSTSYNISITTVSCGKMAAPTRNTCTTGITDPPPPDGSPNITSVSHNSVKVKFSGFEASHGPIKAYAVILTTGEAGHPSADVLKYTYEDFKKGASDTYVTYLIRTEEKGRSQSLSEVLKYEIDVGNESTTLGYYNGKLEPLGSYRACVAGFTNITFHPQNKGLIDGAESYVSFSRYSDAVSLPQDPGVICGAVFGCIFGALVIVTVGGFIFWRKKRKDAKNNEVSFSQIKPKKSKLIRVENFEAYFKKQQADSNCGFAEEYEDLKLVGISQPKYAAELAENRGKNRYNNVLPYDISRVKLSVQTHSTDDYINANYMPGYHSKKDFIATQGPLPNTLKDFWRMVWEKNVYAIIMLTKCVEQGRTKCEEYWPSKQAQDYGDITVAMTSEIVLPEWTIRDFTVKNIQTSESHPLRQFHFTSWPDHGVPDTTDLLINFRYLVRDYMKQSPPESPILVHCSAGVGRTGTFIAIDRLIYQIENENTVDVYGIVYDLRMHRPLMVQTEDQYVFLNQCVLDIVRSQKDSKVDLIYQNTTAMTIYENLAPVTTFGKTNGYIA

SEQ ID NO: 14 - protein, 211 aa, Homo sapiens
MISRMEKMTMMMKILIMFALGMNYWSCSGFPVYDYDPSSLRDALSASVVKVNSQSLSPYLFRAFRSSLKRVEVLDENNLVMNLEFSIRETTCRKDSGEDPATCAFQRDYYVSTAVCRSTVKVSAQQVQGVHARCSWSSSTSESYSSEEMIFGDMLGSHKWRNNYLFGLISDESISEQFYDRSLGIMRRVLPPGNRRYPNHRHRARINTDFE

SEQ ID NO: 15 - protein, 760 aa, Homo sapiens
MMDQARSAFSNLFGGEPLSYTRFSLARQVDGDNSHVEMKLAVDEEENADNNTKANVTKPKRCSGSICYGTIAVIVFFLIGFMIGYLGYCKGVEPKTECERLAGTESPVREEPGEDFPAARRLYWDDLKRKLSEKLDSTDFTGTIKLLNENSYVPREAGSQKDENLALYVENQFREFKLSKVWRDQHFVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKEIKILNIFGVIKGFVEPDHYVVVGAQRDAWGPGAAKSGVGTALLLKLAQMFSDMVLKDGFQPSRSIIFASWSAGDFGSVGATEWLEGYLSSLHLKAFTYINLDKAVLGTSNFKVSASPLLYTLIEKTMQNVKHPVTGQFLYQDSNWASKVEKLTLDNAAFPFLAYSGIPAVSFCFCEDTDYPYLGTTMDTYKELIERIPELNKVARAAAEVAGQFVIKLTHDVELNLDYERYNSQLLSFVRDLNQYRADIKEMGLSLQWLYSARGDFFRATSRLTTDFGNAEKTDRFVMKKLNDRVMRVEYHFLSPYVSPKESPFRHVFWGSGSHTLPALLENLKLRKQNNGAFNETLFRNQLALATWTIQGAANALSGDVWDIDNEF

SEQ ID NO: 16 - protein, 251 aa, Homo sapiens
MAEDLGLSFGETASVEMLPEHGSCRPKARSSSARWALTCCLVLLPFLAGLTTYLLVSQLRAQGEACVQFQALKGQEFAPSHQQVYAPLRADGDKPRAHLTVVRQTPTQHFKNQFPALHWEHELGLAFTKNRMNYTNKFLLIPESGDYFIYSQVTFRGMTSECSEIRQAGRPNKPDSITVVITKVTDSYPEPTQLLMGTKSVCEVGSNWFQPIYLGAMFSLQEGDKLMVNVSDISLVDYTKEDKTFFGAFLL

SEQ ID NO: 17 - protein, 291 aa, Homo sapiens
MQRARPTLWAAALTLLVLLRGPPVARAGASSAGLGPVVRCEPCDARALAQCAPPPAVCAELVREPGCGCCLTCALSEGQPCGIYTERCGSGLRCQPSPDEARPLQALLDGRGLCVNASAVSRLRAYLLPAPPAPGNASESEEDRSAGSVESPSVSSTHRVSDPKFHPLHSKIIIIKKGHAKDSQRYKVDYESQSTDTQNFSSESKRETEYGPCRREMEDTLNHLKFLNVLSPRGVHIPNCDKKGFYKKKQCRPSKGRKRGFCWCVDKYGQPLPGYTTKGKEDVHCYSMQSK

SEQ ID NO: 18 - protein, 461 aa, Homo sapiens
MTPNSMTENGLTAWDKPKHCPDREHDWKLVGMSEACLHRKSHSERRSTLKNEQSSPHLIQTTWTSSIFHLDHDDVNDQSVSSAQTFQTEEKKCKGYIPSYLDKDELCVVCGDKATGYHYRCITCEGCKGFFRRTIQKNLHPSYSCKYEGKCVIDKVTRNQCQECRFKKCIYVGMATDLVLDDSKRLAKRKLIEENREKRRREELQKSIGHKPEPTDEEWELIKTVTEAHVATNAQGSHWKQKRKFLPEDIGQAPIVNAPEGGKVDLEAFSHFTKIITPAITRVVDFAKKLPMFCELPCEDQIILLKGCCMEIMSLRAAVRYDPESETLTLNGEMAVTRGQLKNGGLGVVSDAIFDLGMSLSSFNLDDTEVALLQAVLLMSSDRPGLACVERIEKYQDSFLLAFEHYINYRKHHVTHFWPKLLMKVTDLRMIGACHASRFLHMKVECPTELFPPLFLEVFED

SEQ ID NO: 19 - protein, 115 aa, Homo sapiens
MNAFLLSALCLLGAWAALAGGVTVQDGNFSFSLESVKKLKDLQEPQEPRVGKLRNFAPIPGEPVVPILCSNPNFPEELKPLCKEPNAQEILQRLEEIAEDPGTCEICAYAACTGC

SEQ ID NO: 20 - protein, 131 aa, Homo sapiens
MTPLLTLILVVLMGLPLAQALDCHVCAYNGDNCFNPMRCPAMVAYCMTTRTSAAEAIWCHQCTGFGGCSHGSRCLRDSTHCVTTATRVLSNTEDLPLVTKMCHIGCPDIPSLGLGPYVSIACCQTSLCNHD

SEQ ID NO: 21 - protein, 1606 aa, Homo sapiens
MSEDSRGDSRAESAKDLEKQLRLRVCVLSELQKTERDYVGTLEFLVSAFLHRMNQCAASKVDKNVTEETVKMLFSNIEDILAVHKEFLKVVEECLHPEPNAQQEVGTCFLHFKDKFRIYDEYCSNHEKAQKLLLELNKIRTIRTFLLNCMLLGGRKNTDVPLEGYLVTPIQRICKYPLILKELLKRTPRKHSDYAAVMEALQAMKAVCSNINEAKRQMEKLEVLEEWQSHIEGWEGSNITDTCTEMLMCGVLLKISSGNIQERVFFLFDNLLVYCKRKHRRLKNSKASTDGHRYLFRGRINTEVMEVENVDDGTADFHSSGHIVVNGWKIHNTAKNKWFVCMAKTPEEKHEWFEAILKERERRKGLKLGMEQDTWVMISEQGEKLYKMMCRQGNLIKDRKRKLTTFPKCFLGSEFVSWLLEIGEIHRPEEGVHLGQALLENGIIHHVTDKHQFKPEQMLYRFRYDDGTFYPRNEMQDVISKGVRLYCRLHSLFTPVIRDKDYHLRTYKSVVMANKLIDWLIAQGDCRTREEAMIFGVGLCDNGFMHHVLEKSEFKDEPLLFRFFSDEEMEGSNMKHRLMKHDLKVVENVIAKSLLIKSNEGSYGFGLEDKNKVPIIKLVEKGSNAEMAGMEVGKKIFAINGDLVFMRPFNEVDCFLKSCLNSRKPLRVLVSTKPRETVKIPDSADGLGFQIRGFGPSVVHAVGRGTVAAAAGLHPGQCIIKVNGINVSKETHASVIAHVTACRKYRRPTKQDSIQWVYNSIESAQEDLQKSHSKPPGDEAGDAFDCKVEEVIDKFNTMAIIDGKKEHVSLTVDNVHLEYGVVYEYDSTAGIKCNVVEKMIEPKGFFSLTAKILEALAKSDEHFVQNCTSLNSLNEVIPTDLQSKFSALCSERIEHLCQRISSYKKFSRVLKNRAWPTFKQAKSKISPLHSSDFCPTNCHVNVMEVSYPKTSTSLGSAFGVQLDSRKHNSHDKENKSSEQGKLSPMVYIQHTITTMAAPSGLSLGQQDGHGLRYLLKEEDLETQDIYQKLLGKLQTALKEVEMCVCQIDDLLSSITYSPKLERKTSEGIIPTDSDNEKGERNSKRVCFNVAGDEQEDSGHDTISNRDSYSDCNSNRNSIASFTSICSSQCSSYFHSDEMDSGDELPLSVRISHDKQDKIHSCLEHLFSQVDSITNLLKGQAVVRAFDQTKYLTPGRGLQEFQQEMEPKLSCPKRLRLHIKQDPWNLPSSVRTLAQNIRKFVEEVKCRLLLALLEYSDSETQLRRDMVFCQTLVATVCAFSEQLMAALNQMFDNSKENEMETWEASRRWLDQIANAGVLFHFQSLLSPNLTDEQAMLEDTLVALFDLEKVSFYFKPSEEEPLVANVPLTYQAEGSRQALKVYFYIDSYHFEQLPQRLKNGGGFKIHPVLFAQALESMEGYYYRDNVSVEEFQAQINAASLEKVKQYNQKLRAFYLDKSNSPPNSTSKAAYVDKLMRPLNALDELYRLVASFIRSKRTAACANTACSASGVGLLSVSSELCNRLGACHIIMCSSGVHRCTLSVTLEQAIILARSHGLPPRYIMQATDVMRKQGARVQNTAKNLGVRDRTPQSAPRLYKLCEPPPPAGEE

SEQ ID NO: 22 - protein, 201 aa, Homo sapiens
MKWVWALLLLAALGSGRAERDCRVSSFRVKENFDKARFSGTWYAMAKKDPEGLFLQDNIVAEFSVDETGQMSATAKGRVRLLNNWDVCADMVGTFTDTEDPAKFKMKYWGVASFLQKGNDDHWIVDTDYDTYAVQYSCRLLNLDGTCADSYSFVFSRDPNGLPPEAQKIVRQRQEELCLARQYRLIVHNGYCDGRSERNLL

SEQ ID NO: 23 - protein, 14 aa, Homo sapiens
AGDFLEANYMNLQR

SEQ ID NO: 24 - protein, 6 aa, Homo sapiens
SLYLGR

SEQ ID NO: 25 - protein, 10 aa, Homo sapiens
DLLLPQPDLR

SEQ ID NO: 26 - protein, 10 aa, Homo sapiens
VAAGAFQGLR

SEQ ID NO: 27 - protein, 10 aa, Homo sapiens
VAAGAFQGLR

SEQ ID NO: 28 - protein, 6 aa, Homo sapiens
ELDLSR

SEQ ID NO: 29 - protein, 7 aa, Homo sapiens
LFQGLGK

SEQ ID NO: 30 - protein, 7 aa, Homo sapiens
LFQGLGK

SEQ ID NO: 31 - protein, 8 aa, Homo sapiens
FLNVLSPR

SEQ ID NO: 32 - protein, 11 aa, Homo sapiens
YGQPLPGYTTK

SEQ ID NO: 33 - protein, 7 aa, Homo sapiens
VALTGVR

SEQ ID NO: 34 - protein, 6 aa, Homo sapiens
IYIHPR

SEQ ID NO: 35 - protein, 16 aa, Homo sapiens
SYELPDGQVITIGNER

SEQ ID NO: 36 - protein, 7 aa, Homo sapiens
AWFLESK

SEQ ID NO: 37 - protein, 7 aa, Homo sapiens
AQAWGER

SEQ ID NO: 38 - protein, 7 aa, Homo sapiens
ALDNLAR

SEQ ID NO: 39 - protein, 10 aa, Homo sapiens
EPNAQEILQR

SEQ ID NO: 40 - protein, 7 aa, Homo sapiens
EYEIAIR

SEQ ID NO: 41 - protein, 6 aa, Homo sapiens
TAGLVR

SEQ ID NO: 42 - protein, 7 aa, Homo sapiens
LELHLPK

SEQ ID NO: 43 - protein, 13 aa, Homo sapiens
VLSNTEDLPLVTK

SEQ ID NO: 44 - protein, 6 aa, Homo sapiens
SLLHLK

SEQ ID NO: 45 - protein, 6 aa, Homo sapiens
SLLHLK

SEQ ID NO: 46 - protein, 6 aa, Homo sapiens
AFYLDK

SEQ ID NO: 47 - protein, 10 aa, Homo sapiens
YWGVASFLQK

SEQ ID NO: 48 - protein, 9 aa, Homo sapiens
DALSASVVK

SEQ ID NO: 49 - protein, 7 aa, Homo sapiens
LYWDDLK

SEQ ID NO: 50 - protein, 10 aa, Homo sapiens
SGVGTALLLK

SEQ ID NO: 51 - protein, 10 aa, Homo sapiens
SGVGTALLLK

SEQ ID NO: 52 - protein, 7 aa, Homo sapiens
AHLTVVR

SEQ ID NO: 53 - protein, 7 aa, Homo sapiens
EFQEGLK

SEQ ID NO: 54 - protein, 13 aa, Homo sapiens
AGALNSNDAFVLK

SEQ ID NO: 55 - protein, 11 aa, Homo sapiens
HYGYSLYSAIK

SEQ ID NO: 56 - protein, 9 aa, Homo sapiens
TLTLLSVTR

SEQ ID NO: 57 - protein, 9 aa, Homo sapiens
LLLQPSPQR

SEQ ID NO: 58 - protein, 15 aa, Homo sapiens
QHSVLHLVPINATSK

SEQ ID NO: 59 - protein, 10 aa, Homo sapiens
IPVGPETLGR

SEQ ID NO: 60 - protein, 6 aa, Homo sapiens
ALQVVR

SEQ ID NO: 61 - protein, 7 aa, Homo sapiens
SFAINFK

SEQ ID NO: 62 - protein, 12 aa, Homo sapiens
SFENSLGINVPR

SEQ ID NO: 63 - protein, 14 aa, Homo sapiens
EEVVGLTETSSQPK

SEQ ID NO: 64 - protein, 10 aa, Homo sapiens
HTLNQIDEVK

SEQ ID NO: 65 - protein, 9 aa, Homo sapiens
ISSPTETER

SEQ ID NO: 66 - protein, 9 aa, Homo sapiens
EVELIVQEK

SEQ ID NO: 67 - protein, 11 aa, Homo sapiens
TAASIYEELLK

SEQ ID NO: 68 - protein, 13 aa, Homo sapiens
AGALNSNDAFVLK

SEQ ID NO: 69 - protein, 9 aa, Homo sapiens
HSLVSFVVR

SEQ ID NO: 70 - protein, 9 aa, Homo sapiens
PFDAFTDLK

SEQ ID NO: 71 - protein, 8 aa, Homo sapiens
GKITDLIK